(19) United States
(12) Patent Application Publication
(10) Pub. No.: US 2026/0258368 A1
(43) Pub. Date: Sep. 3, 2026

(54) METABOLIC SELECTION FOR GLYCOGEN-STORING CELLS IN VITRO

(71) Applicant: The Board of Trustees of the Leland Stanford Junior University, Stanford, CA (US)

(72) Inventors: Lay Teng Ang, Jurong (SG); Kevin J. Liu, San Francisco, CA (US); Kyle M. Loh, Piscataway, NJ (US); Nicole Min Qian Pek, Cincinnati, OH (US); Irving L. Weissman, Stanford, CA (US)

(21) Appl. No.: 19/155,524

(22) PCT Filed: Feb. 15, 2024
(86) PCT No.: PCT/US2024/015990

§ 371 Date: Aug. 11, 2025

Related U.S. Application Data
(60) Provisional application No. 63/445,956, filed on Feb. 15, 2023.

(51) Int. Cl. C12N 5/071

(52) U.S. Cl. CPC C12N 5/067; C12N 2500/38; C12N 2501/33; C12N 2501/39; C12N 2501/42; C12N 2501/727

(57) ABSTRACT

Methods are provided for in vitro selection of metabolic selection of metabolically selectable mammalian cells that don't require exogenous glucose and glutamine in culture. Such cells may be glycogen-storing mammalian cells, including without limitation hepatocytes. Metabolic selection provides an approach to rapidly kill non-selectable cells by withholding specific nutrients from the culture medium.

### CROSS REFERENCE TO OTHER APPLICATIONS

This application claims the benefit of U.S. Provisional Application No. 63/445,956, filed Feb. 15, 2023, the contents of which are hereby incorporated by reference in its entirety.

### BACKGROUND

Purifying desired cell-types from heterogeneous cell populations remains a major challenge for stem cell biology and regenerative medicine. In particular, it has been challenging to generate a pure population of liver cells from human pluripotent stem cells. Current approaches produce liver cells alongside non-liver populations. Efficient, cost-effective selection methods are disclosed herein.

### SUMMARY

Methods are provided for the selection, enrichment, and production of metabolically selectable cells, referred to herein as “MESECs” for MEtabolically SElectable Cells. In some apects, MESECs encompass cell types that can surive in culture conditions lacking glucose. In some aspects, MESECs encompass cell types that can survive in culture conditions lacking glucose and glutamine. In some aspects, MESECs encompass cell types that can survive in culture conditions lacking glucose, glutamine, and pyruvate. Cells of interest include, without limitation, hepatocytes, astrocytes, and smooth muscle cells.

Metabolic selection is shown to provide for an essentially pure selected cell population. Importantly, metabolic selection purifies the selected cells based on their metabolic functionality as opposed to surface marker expression. Metabolic selection thus provides a simple, scalable, and inexpensive method for cell purification.

It is shown herein that withholding specific exogenous energy sources, for example glucose, glutamine, pyruvate, etc. for a period of from about 24, and up to about 72 hours, about 4 days, about 5 days, about 6 days, about 7 days, or more leads to the enrichment of MESECs and significantly depletes non-selectable cells. For example, this pulse of nutrient deprivation depletes undifferentiated hPSCs, day 6 liver bud progenitors, and day 6 midgut/hindgut endoderm cells, while sparing, for example, hepatocytes. A preferred combination withdraws both glucose and glutamine, where the level in the culture medium of each of glucose and glutamine is less than about 1 mM, less than about 0.5 mM, less than about 0.1 mM, less than about 10 μM, less than about 1 μM. In some embodiments the medium is also deficient in pyruvate, e.g. less than about 1 mM, less than about 0.5 mM, less than about 0.1 mM, less than about 10 μM, less than about 1 μM.

In some embodiments, a mixed cell population comprising cells for selection are seeded into selection media for culture, in order to select for MESECs over a period of time. The period of time for selection may be, for example, at least about 6 hours, at least about 12 hours, at least 18 hours, at least about 24 hours, at least about 36 hours, at least about 48 hours, and may be about 60 hours, about 72 hours, or more.

In some embodiments, the MESECs comprise cells, including for example hepatocytes, that express metabolic genes integral to glycogen breakdown, for example, PGYL and AGL, glycogen synthesis, for example, GBE1, glutamine synthesis, for example, GS, galactose metabolism for example, GALK1, GALK2, GALE, and GALT, and gluconeogenesis, for example, G6PC, FBP1, and PCK1. In some cases, MESECs, for example, hepatocytes express lower levels of glycolysis genes such as HK1/2 and PKM1/2 than cell types such as hPSCs. In some cases, MESECs comprise cells, such as hepatocytes, that express a wide range of metabolic genes involved in glucose, lipid, and triglyceride metabolism.

In some embodiments a MESEC is a hepatocyte. In some embodiments, a hepatocyte comprises a hepatocyte or hepatocyte like cell is a cell that expresses one or more of, two or more of, three or more of, four or more of, five or more of, or all of ALB, FGB, SERPINA1, CPS1, ARG1, TTR, FAH, and AAT.

Embodiments of the invention include a composition comprising MESECs in selection medium, for example in the presence of non-selectable cells. In some embodiments, a composition of selected MESECs is provided, where the cells have been maintained in culture for at least about 12 hours, at least about 24 hours, at least about 36 hours, at least about 48 hours. In some such embodiments the cell have been genetically modified in culture. In some such embodiments the purified cell population is provided in a pharmaceutically acceptable excipient for transplantation, and may be provided in an effective dose for transplantation, e.g. at least about 10⁵ cells, at least about 10⁶ cells, at least about 10⁷ cells, at least about 10⁸ cells or more.

In some embodiments of the invention a culture medium is provided, which may be referred to herein as selection medium (SM), that allows for viability of MESECs during in vitro culture, but which does not allow for growth of non-selectable cells, including without limitation stem cells, progenitor cells such as liver progenitor cells, etc. For human cells the selection medium is deficient in glucose and glutamine; and optionally further deficient in pyruvate. The medium may comprise “CDM-4” medium. The medium may optionally further comprise one or more of 10 μg/ml Insulin, 10 μM forskolin, 100 μg/ml ascorbic-2-phoshate, 10 μM Dexamethasone, 2 μM Ro4929097 in “CDM-4” media.

In some embodiments of the invention, methods are provided for selection of MESECs, e.g. hepatocytes, astrocytes, smooth muscle cells, etc., the method comprising deriving cells of interest from a suitable progenitor cell, including without limitation human iPSC; and culturing the cells in selection medium for a period of time sufficient to select for the MESECs. In some embodiments the methods further comprise transplanting the cells to an individual. In some embodiments the transplantation is autologous. In other embodiments the selected cells are used in screening assays, for analysis of in vitro activity, and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1G. Transcriptional and chromatin roadmap for human liver differentiation from hPSCs. A) Stepwise differentiation of hPSCs towards hepatocytes (modified from 69), followed by metabolic selection to purify hepatocytes. B) scRNAseq of H1 hPSC differentiated toward hepatocytes (days 1-18 [d1-d18] of differentiation), liver cancer cell lines (HepG2 and HuH7), and freshly-thawed and cultured adult human hepatocytes. C) Flow cytometry reveals generation of MIXL1+ anteriormost primitive streak (assayed by flow cytometry of MIXL1-GFP hPSCs), CXCR4+ definitive endoderm, and ALBUMIN+ hepatocytes within 1, 2 and 18 days of hPSC differentiation, respectively. D) scRNAseq of H1 hPSCs differentiated toward day 6 liver bud progenitors. The entire cell population was harvested without pre-selection. Gene expression quantified in log₂ UMI units. E) scRNAseq of H1 hPSCs differentiated toward day 18 hepatocytes. The entire cell population was harvested without pre-selection. Each column represents the gene expression of a single cell, clustered by differentiation timepoints. F) Bulk-population RNAseq and OmniATACseq of H1 hPSCs differentiated towards hepatocytes. G) Transcription factor motifs enriched within accessible chromatin regions at each stage of H1 hPSC differentiation towards hepatocytes, as determined by OmniATACseq.

FIGS. 2A-2F. Intestinal cells emerge alongside hPSC-derived hepatocytes and can be removed using cell-surface marker CD200. A) ALBUMIN (ALB), CDX2, and PDX1 immunostaining of H1 hPSC-derived day 18 hepatocyte-containing populations, followed by image quantification. Scale bar, 100 μm. Top right represents a magnification of the top left brightfield image. B) Hnf4a and Cdx2 mRNA staining of a mouse embryo at embryonic day 9.5 (E9.5) by HCR3.0, showing close proximity of developing liver and intestines. C) scRNAseq of day 18 H1 hPSC-derived hepatocyte-containing populations, followed by Louvain clustering to identify multiple cell-types within the population. The entire cell population was harvested without pre-selection. Gene expression quantified in log₂ UMI units. D) High throughput antibody screening of FAH-2A-Clover reporter hPSCs, which were either undifferentiated, differentiated for 18 days (“unsorted hepatocytes”), or day 18 populations that were subset into Clover+vs. Clover-subpopulations. E) Flow cytometry of CD200 expression on H1 hPSCs before or after differentiation into day 18 hepatocytes. F) qPCR of CD200 hi vs. CD200l populations isolated from day 18 hPSC-derived hepatocyte populations by fluorescence-activated cell sorting (FACS), as well as undifferentiated hPSCs as a negative control. * P<0.05, ** P<0.01.

FIGS. 3A-3E. Metabolic selection eliminates non-liver cells, thereby purifying hPSC-derived hepatocytes. A) qPCR of undifferentiated H1 hPSCs, day 6 liver bud progenitors, and day 18 hepatocytes reveals the upregulation of metabolic pathway genes in hepatocytes. B) ALBUMIN, CDX2, and PDX1 immunostaining of H1 hPSC-derived day 18 hepatocytes cultured in standard vs. metabolic selection media for 3 additional days. Scale bar, 100 μm. C) qPCR of H1 hPSC-derived hepatocytes cultured in standard vs. metabolic selection media for 3 days, with undifferentiated hPSCs shown as a negative control. D) Cell viability staining of undifferentiated hPSCs, day 6 liver bud progenitors, and day 18 hepatocytes cultured in standard vs. metabolic selection media for 1-2 days. Scale bar, 500 μm. E) scRNAseq of H1 hPSC-derived hepatocytes cultured in metabolic selection media for 2 days reveals near-absence of non-liver marker expression. The entire cell population was harvested, without pre-selecting cell subsets. Gene expression quantified in log₂ UMI units.

FIGS. 4A-4E. Comparison of metabolically-selected hPSC-derived hepatocytes, adult primary hepatocytes, and liver cancer cells. A) scRNAseq of metabolically-selected H1 hPSC-derived hepatocytes, adult primary hepatocytes that were either freshly isolated or cultured for 6 days, and liver cancer cell lines (HepG2 and HuH7). Each column represents the gene expression of a single cell, clustered together by cell-type. B) Average expression levels of prevailing hepatocyte marker signature genes across metabolically-selected H1 hPSC-derived hepatocytes, freshly-thawed and cultured adult primary hepatocytes, and liver cancer cell lines (HepG2 and HuH7), as quantified by scRNAseq. C) Quantification of glycogen and glycerol levels across metabolically-selected H1 hPSC-derived hepatocytes, cultured adult primary hepatocytes, and liver cancer cell lines (HepG2 and HuH7). D) Oil Red O staining of H1 hPSC-derived hepatocytes differentiated in either low glucose media vs. high glucose and lipid-rich media for 12 days. E) H1 hPSC-derived hepatocytes were intrasplenically injected into Fah−/− Rag2−/−112rg−/− mice, and 6 weeks later, the liver was stained with a human albumin-specific antibody, revealing engraftment of hPSC-derived hepatocytes.

FIGS. 5A-5B. hPSC-derived and adult hepatocytes activate interferon signaling in response to poly(I:C), but liver cancer cells do not. A) qPCR of hPSC-derived hepatocytes (generated from H1 or H7 hPSC lines), adult primary human hepatocytes (procured from Lonza [L] or Gibco [G]), or liver cancer cells (HepG2 or HuH7) treated with 50 μg/mL poly(I:C) or control media for 1 day. Gene expression data normalized to YWHAZ, such that YWHAZ expression=1.0. Expression of interferon-stimulated genes is shown. B) RNA-seq of hPSC-derived hepatocytes (generated from H1 or H7 hPSC lines), adult primary human hepatocytes (procured from Lonza [L] or Gibco [G]) or liver cancer cells (HepG2 or HuH7) treated with 50 μg/mL poly(I:C) or control media for 1 day.

FIGS. 6A-6E. Ebola virus and Lassa virus infect purified hPSC-derived hepatocytes in vitro and non-human primate hepatocytes in vivo. A) hPSC-derived hepatocytes were purified by metabolic selection, and then cryopreserved, thawed, and cultured for several days before viral inoculation under BSL4 containment. B) hPSC-derived hepatocytes were infected with 10⁵ focus-forming units (FFU) of Ebola virus-GFP, Sudan virus-zsGreen, Marburg virus-zsGreen, or Lassa virus-zsGreen under BSL4 containment. Afterwards, quantification of infectious virus particles in the culture media was performed using the FFU assay, by inoculating Vero cells with hepatocyte-conditioned media. C) Timecourse imaging of hPSC-derived hepatocytes (purified by metabolic selection) infected by 10⁵ FFU of Ebola virus-GFP, Sudan virus-ZsGreen, Marburg virus-ZsGreen, or Lassa virus-ZsGreen. Scale bar, 400 μm. D) albumin immunostaining of hPSC-derived hepatocytes infected with Ebola virus-GFP, Sudan virus-zsGreen, Marburg virus-zsGreen, or Lassa virus-zsGreen for 3 days. Scale bar, 100 μm. E) Rhesus or cynomolgus macaques were infected with Ebola or Lassa viruses under BSL4 containment. Liver tissues were immunolabeled for respective viral antigens (purple) and hepatocyte marker HepPar1/CPS1 (yellow), with colocalization of antigens appearing orange or red.

FIGS. 7A-7F. Ebola virus and Lassa virus fail to induce interferon signaling in purified hPSC-derived hepatocytes. A) Metabolically-selected hPSC-derived hepatocytes were infected with wild-type Ebola virus (Mayinga isolate), wild-type Lassa virus (Josiah isolate), or Sendai virus (Cantell strain). High titers of Ebola or Lassa virus were used to synchronously infect most cells (MOI≈4). B) Intracellular levels of Ebola and Lassa viral RNA after inoculating H1 hPSC-derived, metabolically-selected hepatocytes with Ebola or Lassa virus, as quantified by qPCR of the cell monolayer (left; after removing the culture media) or the supernatant (right). Viral NP gene copy numbers were respectively normalized to the number of hepatocytes plated for the experiment (left), or volume of the cell culture media (right). C) Percentage of viral reads in the transcriptome of hPSC-derived hepatocytes infected by Ebola or Sendai virus, as determined by RNA-seq. Lassa virus reads were not quantified, as Lassa mRNAs are not polyadenylated and thus were not expected to be captured by our RNA-seq strategy. D) Quantification of cell death of hPSC-derived hepatocytes, either infected by Ebola, Lassa, or Sendai virus, or uninfected. An adenylate kinase assay was performed on the culture media. Adenylate kinase is an intracellular enzyme released into the media upon cell death. E) IFNβ secretion by uninfected hPSC-derived hepatocytes, or those infected by Ebola, Lassa, or Sendai virus, as quantified using an enzyme-linked immunosorbent assay (ELISA). F) Bulk population RNA-seq of uninfected hPSC-derived hepatocytes, or those infected by Ebola, Lassa, or Sendai virus, showing expression of interferon-stimulated genes.

FIGS. 8A-81. Ebola virus infection activates WNT and ISR pathways in purified hPSC-derived hepatocytes, whereas Lassa virus does not. A) Volcano plots of differentially-expressed genes in Ebola, Lassa, or Sendai virus-infected hepatocytes, relative to mock-infected controls at the same timepoints. In each volcano plot, genes on the left vs. right represent those downregulated or upregulated by viral infection, respectively. n.d.=not determined. B) RNA-seq comparison of Ebola or Lassa virus-infected hPSC-derived hepatocytes vs. mock-infected hepatocytes to determine gene ontology (GO) terms associated with Ebola or Lassa virus infection. C) Venn diagram of genes differentially or commonly up-or downregulated by Ebola and Lassa virus 2 days post-infection of purified hPSC-derived hepatocytes. D-G) Bulk population RNA-seq of purified hPSC-derived hepatocytes, either infected by Ebola, Lassa, and Sendai virus, or uninfected. H) Bulk population RNA-seq of primate liver tissue infected by Ebola over a time course of 0, 2, 4, and 6 days. I) Summary of purified hPSC-derived hepatocyte responses to viral infection.

FIGS. 9A-9D. Stepwise changes in gene expression during human liver differentiation from hPSCs, related to FIG. 1. A) Quality control statistics for scRNAseq analysis, showing the number of genes expressed per cell and the percentage of mitochondrial reads over total reads per cell for hPSCs (DO) differentiated into hepatocytes (days 1-18 [d1-d18] of differentiation), HepG2, HuH7, and fresh primary human hepatocytes (1° Hep). B) Bulk-population RNA-seq of H1 hPSCs differentiated into hepatocytes, showing stepwise changes in transcription factor expression. Gene expression quantified in scaled count units. Cell-types profiled include undifferentiated hPSCs (UD), day 1 anteriormost primitive streak, day 2 definitive endoderm, day 3 posterior foregut, day 6 liver bud progenitors, day 12 early hepatocytes, and day 18 hepatocytes. C) scRNAseq of hPSCs (DO) differentiated towards early hepatocytes (days 1-12 [d1-d12] of differentiation) to assess the presence of potential population heterogeneity at each step of differentiation. Louvain clustering was applied to decompose each cell population into subclusters, and differentially-expressed genes that distinguish these subclusters were identified. Gene expression quantified in log₂ UMI units. D) Bulk-population RNA-seq of H1 hPSCs, day 1 anteriormost primitive streak, day 2 definitive endoderm, day 3 posterior foregut, day 6 liver bud progenitors, day 12 early hepatocytes, and day 18 hepatocytes, highlighting cell-type-specific gene expression.

FIGS. 10A-10G. Discovery of non-liver cells generated during liver differentiation from hPSCs, related to FIG. 2. A) ALBUMIN, ZO1, CDX2, PDX1, and DAPI immunostaining of H1 hPSC-derived day 18 hepatocyte-containing populations. Scale bar, 50 μm. B) scRNAseq of day 18 H1 hPSC-derived hepatocyte-containing populations, followed by Louvain clustering to identify multiple cell-types within the population. The entire cell population was harvested without pre-selection. In the heatmap, each column represents gene expression of a single cell, clustered together by “cell-type” clusters (top). The proportions of each identified cell-type within the day 18 population are also shown (bottom). Three subsets of non-liver cells were discovered: intestinal goblet, intestinal enteroendocrine, and mesenchymal cells. Markers of different intestinal enteroendocrine cell subtypes were also quantified (right), revealing expression of K/L and X enteroendocrine subtype markers. Gene expression quantified in log₂ UMI units. C) scRNAseq of adult human liver reveals the absence of CD200 expression. D) Bulk-population RNA-seq of different human adult tissues reveals the absence of CD200 mRNA expression from the liver. E) Flow cytometry of CD200 expression on undifferentiated hPSCs, day 2 definitive endoderm, day 6 liver bud progenitors, day 12 early hepatocytes, and day 18 hepatocytes, showing median fluorescence intensity (left) and population expression levels (right). F) Flow cytometry of day 18 hPSC-derived hepatocyte-containing populations, showing FACS gates used to sort CD200 hi vs. CD2001° populations that were analyzed by qPCR in FIG. 2f. G) scRNAseq of hPSC-derived day 18 hepatocyte-containing populations that were computationally binned based on CD200 mRNA expression into CD200 hi vs. CD200° subsets.

FIGS. 11A-11F. Optimization of metabolic selection to purify hPSC-derived hepatocytes, related to FIG. 3. A) Hypothesized mechanisms of metabolic selection. B) Bulk-population RNA-seq of genes upregulated in day 18 H1 hPSC-derived hepatocytes over undifferentiated hPSCs, followed by gene ontology (GO) analysis of hepatocyte-upregulated genes. This revealed transcriptional upregulation of various metabolic pathways in hepatocytes. C) Cell viability staining of undifferentiated hPSCs, day 6 liver progenitors, day 6 midgut/hindgut (posterior) endoderm, and day 18 hepatocytes cultured in standard vs. metabolic selection media for 1-2 days. Scale bar, 500 μm. D) Albumin, CDX2, PDX1, DAPI immunostaining of H1 hPSC-derived day 18 hepatocytes cultured in standard vs. metabolic selection media for 2 additional days. E) Brightfield images of day 18 H1 hPSC-derived hepatocytes that were cultured for 2-3 additional days in metabolic selection media, showing cell death after metabolic selection. F) Bulk-population RNA-seq of day 18 H1 hPSC-derived hepatocytes before, or after culture in metabolic selection media for 1 day to identify genes induced or repressed by metabolic selection.

FIGS. 12A-12D. Additional comparisons of metabolically-selected hPSC-derived hepatocytes, adult primary hepatocytes, and liver cancer cells, related to FIG. 4. A) Bulk-population RNA-seq of metabolically-selected hPSC-derived hepatocytes, HepG2, and HuH7 cells. Gene expression normalized to levels found in hPSC-derived hepatocytes. B) Global transcriptome comparison of metabolically-selected hPSC-derived hepatocytes, cultured primary hepatocytes, HepG2, and HuH7 with freshly thawed adult primary hepatocytes. Transcriptome-scale Pearson correlation coefficients between each pair of cell-types are shown. C) Bulk population RNA-seq performed of primary adult human hepatocytes were cultured for 1-7 days in monolayers. D) scRNAseq analysis of metabolically-selected H1 hPSC-derived hepatocytes, liver cancer cell lines (HepG2 and HuH7), and freshly thawed and cultured adult human hepatocytes. E) H1 hPSC-derived hepatocytes were intrasplenically injected into Fah−/− Rag2−/−112rg−/− mice. 6 weeks later, hPSC-derived hepatocytes that expressed albumin and glutamine synthetase were localized around central veins (CV, left). Others expressed FAH and were localized around bile ducts and portal veins (PV, right). Scale bar: 50 μm (left) and 100 μm (right).

FIGS. 13A-13C. Cryopreservation, thawing, and optimized in vitro culture of hPSC-derived hepatocytes, related to FIG. 6. A) qPCR of H1 hPSC-derived day 18 hepatocytes that were cryopreserved, and then thawed at a density of 150,000 cells/well of a 24-well plate either in standard media or media supplemented with hepatocyte-specifying signals (DFRAI: dexamethasone+ forskolin+RO4929097+ ascorbic acid-2-phosphate [AA2P]+insulin (69)) for 1 week. Gene expression data normalized to YWHAZ, such that YWHAZ expression=1.0. B) qPCR of H1 hPSC-derived day 18 hepatocytes that were cryopreserved, then thawed at a density of 100,000-500,000 cells/well of a 24-well plate in DFRAI media for 1 week. Gene expression data normalized to YWHAZ, such that YWHAZ expression=1.0. C) qPCR of undifferentiated H1 hPSCs, day 18 hPSC-derived hepatocytes before freeze, or hPSC-derived hepatocytes that had been thawed and cultured for 3, 5, or 7 days (D3-7) in DFRAI media at a density of 750,000 cells/well of a 24-well plate. Gene expression data normalized to YWHAZ, such that YWHAZ expression=1.0.

FIGS. 14A-14E. Ebola, Sudan, Marburg, and Lassa virus extensively infect purified populations of hPSC-derived hepatocytes, related to FIG. 6. A) Genome structures of recombinant GFP-expressing Ebola virus, ZsGreen-expressing Sudan virus, ZsGreen-expressing Marburg virus, and ZsGreen-expressing Lassa virus. B) Additional image of a Ebola virus-infected rhesus macaque liver immunolabeled for Ebola virus VP40 and hepatocyte marker HepPar1/CPS1. This rhesus macaque was from the same study shown in FIG. 6e. C) Isotype control immunostaining of hPSC-derived hepatocytes infected with Ebola virus-GFP. Scale bar 100 μm. Negative control for FIG. 6d. D) Relative infectivity of Vero E6 cells vs. H7 hPSC-derived hepatocytes infected with either Ebola virus-GFP, Sudan virus-zsGreen, Marburg virus-zsGreen, or Lassa virus-zsGreen. Both cell-types were inoculated with the same viral dose, and infected cells were quantified using the fluorescent FFU assay. Higher numbers indicate that hPSC-derived hepatocytes were preferentially infected by the virus, relative to Vero cells. E) Timecourse imaging of hPSC-derived hepatocytes (purified by metabolic selection) infected by 103 FFU of Ebola virus-GFP, Sudan virus-ZsGreen, Marburg virus-ZsGreen, or Lassa virus-ZsGreen. Scale bar, 400 μm.

FIGS. 15A-15E. Transcriptional effects of Ebola, Lassa, and Sendai virus on purified hPSC-derived hepatocytes, related to FIG. 7. A) Brightfield images of uninfected hPSC-derived hepatocytes, or those infected by Ebola, Lassa, or Sendai virus. Red arrows indicate overt cell death after Ebola virus infection. B) RNA-seq comparison of Sendai virus-vs. mock-infected hPSC-derived hepatocytes to determine gene ontology (GO) terms associated with Sendai virus-upregulated genes. C) Bulk population RNA-seq of uninfected hPSC-derived hepatocytes, or those infected by Ebola, Lassa, and Sendai virus, showing expression of genes linked to the unfolded protein response and integrated stress response. D) Bulk population RNA-seq of uninfected hPSC-derived hepatocytes, or those infected by Ebola, Lassa, and Sendai virus, showing expression of liver function genes. E) Bulk population RNA-seq of primate liver tissue infected by Ebola over a time course of 0, 2, 4, and 6 days.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The scope of the invention encompasses methods for the selection, enrichment, and production of metabolically selectable cells, referred to herein as “MESECs” for MEtabolically \vec{SE}lectable Cells. In some apects, MESECs encompass cell types that can surive in culture conditions lacking glucose. In some aspects, MESECs encompass cell types that can survive in culture conditions lacking glucose and glutamine. In some aspects, MESECs encompass cell types that can survive in culture conditions lacking glucose, glutamine, and pyruvate.

In some embodiments, the MESECs comprise cells, such as hepatocytes, which express metabolic genes integral to glycogen breakdown, for example, PGYL and AGL, glycogen synthesis, for example, GBE1, glutamine synthesis, for example, GS, galactose metabolism for example, GALK1, GALK2, GALE, and GALT, and gluconeogenesis, for example, G6PC, FBP1, and PCK1. In some cases, MESECs, for example, hepatocytes express lower levels of glycolysis genes such as HK1/2 and PKM1/2 than cell types such as hPSCs. In some cases, MESECs comprise cells, such as hepatocyte, that express a wide range of metabolic genes involved in glucose, lipid, and triglyceride metabolism, indicating that they can use alternative substrates such as lipids and triglycerides to survive a starvation episode.

A cell type's “ability to surive culture conditions” means that for a plurality of cells of that cell type, a substantial portion will survive culture in those conditions for a selected period of time. In various implementations, the substantial portion may comprise at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The selected periof of time may comprise a period of at least 12 hours, at least 24 hours, at least 36 hours, at least 48 hours, at least 60 hours, at least 72 hours, at least 4 days, at least 5 days, at least 6 days, at least 7 days, or longer.

In one implementation, the MESECs comprise hepatocytes. As used herein, “hepatocytes” means cells that are hepatocytes (i.e. primary cells isolated from an animal or explant of an animal, or the cultured progeny of such cells), and cells that are indistinguishable from primary hepatocytes or their progeny. In some embodiments, the hepatocytes encompass heptatocyte-like cells. Hepatocyte-like cells are cells that share one or more attributes with natural hepatocytes.

Glycogen-storing is one of the qualities of liver cells. Besides that, the ability of hepatocytes to produce glucose or energy from non-glucose sources is also key. For example, hepatocytes can metabolise pyruvate, lactate, amino acids, fatty acids etc to produce ATP through gluconeogenesis, ketosis, fatty acid oxidation and other processes. Metabolic selection provides an approach to rapidly kill non-glycogen storing cells by withholding specific nutrients from the culture medium. Glycogen-storing cells, e.g. hepatocytes, are capable of converting glucose into glycogen, and can therefore rely on glycogen during glucose deprivation. Withholding glucose, pyruvate, and glutamine for a short period of time, e.g. from about 1 to 3 days, kills contaminating non-glycogen-storing cells, while the glycogen-storing cells survive.

Attributes of hepatocytes include morphological attributes, functional attributes, and the expression of markers expressed in natural hepatocytes. Hepatocytes (liver cells) comprise up to 80% of the total cell population and volume of the human liver and are intimately associated with both arterial and venous blood. In vivo each hepatocyte is bathed in blood along multiple surfaces via a system of highly fenestrated vessels that enable the bidirectional, cell-to-plasma exchange of components. This physical intimacy facilitates two central functions of the liver in its role as a vital hematological filter: the production of blood plasma proteins and the concomitant endocytic uptake of lipids, growth factors, and other trophic agents.

Attributes of hepatocytes include apical (canalicular) and basolateral (sinusoidal) plasma membrane domains composed of distinct surface proteins, channels, and receptors. Within the sinusoidal domain reside a host of different receptor tyrosine kinases such as the EGF receptor, key lipid- and iron-scavenging receptors such as the low-density lipoprotein receptor (LDLR) and transferrin receptor (TfR), as well as numerous bile acid uptake transporters. In the canalicular domain, ATP-binding cassette (ABC) transporters and other bile acid efflux transporters predominate. Hepatocytes are unique in that they may have several basolateral and apical domains in close proximity.

Attributes of hepatocytes include secretion of α-fetoprotein, albumin, transferrin, plasminogen, fibrinogen, and clotting factors. Synthesized only by hepatocytes, serum albumin is the most highly secreted protein of any cell. In addition to the significant complement of proteins exported from the hepatocyte, an attribute of hepatocytes is a unique polarity via the selective routing of resident membrane proteins to either the sinusoidal or canalicular membranes; which critical for the production of bile, a complex molecular soap composed mainly of cholesterol, phospholipids (predominantly phosphatidylcholine), electrolytes, conjugated bilirubin, and bile acids. Bile synthesis begins in the hepatocyte and its components are transported across the apical membrane into the bile canaliculi formed between adjacent hepatocytes. The hepatic bile duct empties into the gall bladder, where bile is ultimately concentrated and stored until its hormonally stimulated release into the intestine via the common bile duct.

Attributes of hepatocytes include a basolateral membrane, which forms the sinusoidal surface that directly contacts portal blood. In addition to receptor tyrosine kinases and trophic receptors, the sinusoidal surface contains a subset of ABC transporters and solute transporters that mediate retrieval of bile acids and other biliary components from the circulation. The sodium taurocholate cotransporter (NTCP) is the major mechanism for retrieval of conjugated (water-soluble) bile acids from the portal blood, whereas several members of the organic anion-transporting polypeptides are responsible for retrieving unconjugated (water-insoluble) bile acids. Organic anion transporters facilitate uptake of prostaglandin and drugs from the circulation, while organic cation transporters retrieve small organic cations. MRP3/ABCC3, MRP4/ABCC4, and MRP6/ABCC6 mediate efflux of bile components into the blood, and expression of both MRP3 and MRP4 is enhanced by cholestasis.

Additional attributes of hepatocytes are attributes of the sinusoidal membrane. The, proteins present at the sinusoidal membrane are critical for the hepatocyte to internalize factors from the blood. The liver has evolved into a biological filter used to remove and process dietary nutrients (e.g., glucose, lipids, and iron) as well as toxins that could damage organs without detoxification capabilities. Hepatocytes internalize many of these extracellular materials via endocytosis. A variety of endocytic mechanisms have been described in the hepatocyte, including fluid-phase and caveolae-based endocytosis, the most active and well defined being receptor-mediated endocytosis. Once inside the cell, the endocytosed receptors, ligands, and cargo proteins are sorted and trafficked to a variety of destinations, recycled back to the plasma membrane, or degraded within multivesicular bodies, late endosomes, and lysosomes. An additional major function of the liver is the systemic homeostasis of iron. In addition to iron uptake, hepatocytes also govern the release of iron into the bloodstream by signaling to Kupffer cells and other circulating macrophages that are rich in stored iron.

Another attribute of hepatocytes is glycogen storage. Insulin binds to insulin receptors to signal the storage of dietary nutrients such as glucose and fatty acids within the liver, adipose, muscle, and other tissues. Glucose storage by the liver is particularly important to glucose homeostasis, and hepatocytes can store glucose within branched chains of glycogen polysaccharides.

In some embodiments, the hepatocyte comprises a hepatocyte or hepatocyte like cell that expresses one or more markers of hepatocyte identity, for example, mature hepatocyte identity. Exemplary markers of hepatocyte identity include ALB, FGB, SERPINA1, CPS1, ARG1, TTR, FAH, and AAT. In some embodiments, the hepatocyte comprises a hepatocyte or hepatocyte like cell that expresses one or more of, two or more of, three or more of, four or more of, five or more of, or all of ALB, FGB, SERPINA1, CPS1, ARG1, TTR, FAH, and AAT.

In some embodiments, the hepatocyte comprises a hepatocyte or hepatocyte like cell that has been derived in culture from a different cell type. In a primary embodiment, the hepatocytes or hepatocyte like cells include cells that have been differentiated from pluripotent cells.

In some embodiments, the MESECs comprise an astrocyte. As used herein, an astrocyte refers to a cell, including a culture cell, that has substantial morphological or functional attributes of in-vivo astrocytes, including protoplasmic astrocytes and fibrous astrocytes. In some embodiments, the morphological attributes comprise multiple processes. In some embodiments, the astrocyte comprises a cell that expresses two or more of, three or more of, four or more of, five or more of, six or more of, seven or more of, or eight or more of astrocyte markers, for example makers selected from the group consisting of A2B5, aldolase C, astrocytomas, aquaporin-4, glial fibrillary acidic protein (GFAP), excitatory amino acid transporter 1 (EAAT1), also known as GLAST-1, excitatory amino acid transporter 1 (EAAT2), also known as glutamate transporter 1 (GLT-1), glutamine synthetase, S100 beta, aldehyde dehydrogenase 1 family member L1 (ALDH1L1), connexin 43/GJA1, HES-1, NDRG-2, PEA-15, Sox9, and survivin.

In some embodiments, the astrocyte comprises an astrocyte has been derived in culture from a different cell type. In a primary embodiment, the astrocytes comprise an astrocyte that has been differentiated from pluripotent cells.

In some embodiments, the MESEC comprises a smooth muscle cell. As used herein, an smooth muscle cell refers to a cell, including a culture cell, that has substantial morphological or functional attributes of in-vivo smooth muscle cells. Smooth muscle cell attributes include a spindle shap, having a single centrally located nucleus, lacking transverse striations, having no T tubules, having numerous caveolae, and having an extensive contractile apparatus on its cell membrane over a substantial portion of the cell length. In some embodiments, the smooth muscle cell comprises a cell that expresses two or more of, three or more of, four or more of, five or more of, six or more of, seven or more of, or eight or more of smooth muscle cell markers, for example makers selected from the group consisting of smoothelin, ACTA2, CNN1, TAGLN, TAGLN2, alpha-Smooth Muscle Actin, VE-Cadherin, Caldesmon/CALD1, Calponin 1, EMILIN2, Hexim 1, Histamine H2 R, and Motilin R/GPR38.

In some embodiments, the smooth muscle cell comprises a smooth muscle cell that has been derived in culture from a different cell type. In a primary embodiment, the smooth muscle cell comprises a cell that has been differentiated from pluripotent cells.

In some embodiments, the MESEC comprises a cell that has been derived in culture from a different cell type. In a primary embodiment, the MESEC comprises a cell that has been differentiated from pluripotent cells.

The terms “pluripotent cells,” “pluripotent progenitor cells”, “pluripotent progenitors”, “pluripotent stem cells”, “multipotent progenitor cells” and the like, as used herein refer to cells that are capable of differentiating into two or more different cell types and proliferating. Non limiting examples of pluripotent precursor cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like. Pluripotent progenitor cells may be acquired from public or commercial sources or may be newly derived.

The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or asymmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive

The terms “grafting”, “engrafting”, and “transplanting” and “graft” and “transplantation” as used herein refer to the process by which stem cells or other cells according to the present disclosure are delivered to the site where the cells are intended to exhibit an effect, such as, but not limited to, repairing damage to a patient's central nervous system, treating autoimmune diseases, treating diabetes, treating neurodegenerative diseases, or treating the effects of nerve, muscle and/or other damage caused by birth defects, stroke, cardiovascular disease, a heart attack or physical injury or trauma or genetic damage or environmental insult to the body, caused by, for example, disease, an accident or other activity. The selected cells of the present disclosure can also be delivered in a remote area of the body by any mode of administration as described herein. For example, the term “cell engraftment” as used herein can refer to the process by which cells such as, but not limited to, muscle stem cells, are delivered to, and become incorporated into, a differentiated tissue such as a muscle, and become a part of that tissue. For example, muscle stem cells, when delivered to a muscle tissue, may proliferate as stem cells, and/or may bind to skeletal muscle tissue, differentiate into functional myoblasts cells, and subsequently develop into functioning myofibers. Transplantation may utilize a dose of cells effective to obtain the desired effect, which may be delivered in an appropriate medium or substrate.

The MESEC cells are preferably human but can also be non-human, e.g., from non-human mammals. Examples of non-human mammals include, but are not limited to, non-human primates (e.g., apes, monkeys, gorillas), rodents (e.g., mice, rats), cows, pigs, sheep, horses, dogs, cats, or rabbits. Similarly, the cell can be from any organism, reptile, microbe, or microorganism. The cells may be derived from a human subject or human patient. The subject may be free of a disease or disorder, or the subject may suffer from a disease or disorder, or at risk for such disease or disorder. The subject may be a female; in some cases, the subject is a male.

In some embodiments, the MESEC comprises a modified cell. Methods of modification of cells, including modification of pluripotent cells and modification of hepatocytes are well-known in the art and include but are not limited to e.g., genetic modification (e.g., through deletion mutagenesis, through substitution mutagenesis), through insertional mutagenesis (e.g., through the introduction of heterologous nucleic acid into the pluripotent cell, etc.), non-mutagenic genetic modification (e.g., the non-mutagenic insertion of heterologous nucleic acid, etc.), epigenetic modification (e.g., through the treatment with one or more specific or general epigenetic modifying agents (e.g., methylation inhibitors, methylation activators, demethylases, etc.), other modifications (e.g., non-genetic labeling, etc.).

Modifications of cells may be transient or stable. In some instances, a modification of a particular pluripotent cell or hepatocyte may be stable such that the modification persists through selection of a desired cell type from the mixed population as described herein. In some instances, stable modifications may persist through introduction into a host. In some instances, stable modifications may persist through proliferation of the cell such that all progenitors of a particular modified cell also contain the subject modification.

Various embodiments described herein encompass the process of culturing cells in a culture medium. For example, common cell culture processes encompass growing or maintaining populations of cells in in vitro in an appropriate liquid nutrient medium. Generally, the seeding level will be at least about 10 cells/ml, more usually at least about 10⁰ cells/ml and generally not more than about 10⁵ cells/ml, usually not more than about 10⁴ cells/ml. Cells may be cultured singly or in groups.

Various media are commercially available and may be modified for the invention, for example, modified to comprise a selection medium as described herein. Commercial media include Ex vivo serum free medium, Dulbecco's Modified Eagle Medium (DMEM), MCDB, RPMI, Iscove's medium, etc. Appropriate antibiotics to prevent bacterial growth and other additives, including, but not limited to, pyruvate (0.1-5 mM), glutamine (0.5-5 mM), 2-mercaptoethanol (1-10×10⁻⁵ M) may also be included. The medium may be any conventional culture medium.

The term “cell culture” or “culture” means the maintenance of cells in an artificial, in vitro environment. Culture conditions may include, without limitation, a specifically dimensioned container, e.g. flask, roller bottle, plate, 96 well plate, etc.; culture medium comprising suitable factors and nutrients for growth of the desired cell type; and a substrate on the surface of the container or on particles suspended in the culture medium. By “container” is meant a glass, plastic, or metal vessel that can provide an aseptic environment for culturing cells.

In some embodiments, the cells are cultured on a solid substrate, for example, naturally-derived or synthetic substrates, such as extracellular matrix materials, includingcollagen, fibronectin, laminin. poly-lysine, and mixtures of components, for example, GELTREX™ (Thermoi Fisher Scientific), which comprises a mixture of laminin, collagen IV, entactin, and heparin sulfate proteoglycans. In other implementations, the cells are cultured on a substrate comprising a cellular feeder layer, such as MEF's, human dermal fibroblasts, Adipose-derived mesenchymal stem cells, 3T3 cells, and others known in the art.

In some embodiments, the cultured cells are grown in an organoid, three dimensional, organotypic, micro-tissue, and like systems for recapitulating aspects of in-vivo functional tissues. For example, hepatic organoid systems known in the art . . . .

### Methods

The present disclosure provides methods and compositions for metabolic selection of cultured cells. As used herein, “perfroming metabolic selection,” “applying metabolic selection,” “culturing under metabolic selection,” and like terms refers to the process of culturing a population of cells, wherein a portion of the population comprises MESECs and a portion of the population comprises non-MESEC cells under conditions that include: conditions lacking glucose; conditions lacking glucose and glutamine; or conditions lacking glucose, glutamine, and pyruvate, for a period of time such that a substantial portion of the population of non-MESEC cells dies and a substantial portion of the resulting population comprises MESECs.

As used herein, a conditionals lacking a specified nutrient refers to culture conditions where that nutrient is present in limiting concentrations insufficient to support the survival of a substantial portion of non-MESEC cells in a population. Culture conditions refers to the environment in which the cells are cultured. In a primary implementation, metabolic selection is applied by culturing the cells in a culture medium that is lacking glucose; glucose and glutamine; or glucose, glutamine, and pyruvate. In some implementations, the specified nutrient(s), for example: glucose; glucose and glutamine; or glucose, glutamine, and pyruvate, are essentially absent from the culture conditions, i.e. absent from the culture medium and culture substrate, if any. In embodiments, any of glucse, glucose and glutamine, or glucose, glutamine, and pyruvate are present in the culture conditions (i.e. culture medium) at concentations of less than about 1 mM, less than about 0.5 mM, less than about 0.1 mM, less than about 10 μM, less than about 1 μM, less than 100 nM, less than 10 nM, or less than 1.0 nM.

In various embodiments, the scope of the invention encompasses a selection medium. The selection medium comprises a culture medium that is lacking glucose; lacking glucose and glutamine; or lacking glucose, glutamine, and pyruvate. The selection medium of the invention may comprise any cell culture medium known in the art which is lacking glucose; lacking glucose and glutamine; or lacking glucose, glutamine, and pyruvate, or which has been modified such that it is lacking glucose; lacking glucose and glutamine; or lacking glucose, glutamine, and pyruvate. In some embodiments, the culture medium comprises HepSelect Media. The composition of HepSelect basal medium is as follows: 100% Dulbecco's Modified Eagle Medium (DMEM) lacking glucose, glutamine, phenol red, and pyruvate+15 μg/mL human transferrin+1% v/v penicillin/streptomycin. In some embodiments the HepSelect medium is supplemented with one or more of: Forskolin (for example, at about 10 UM), Dexamethasone (for example, at about 10 μM), RO4929097 (for example, at about 2 μM), AA2P (for example, at about 200 μg/mL), and Insulin (for example, at about 10 μg/mL).

Metabolic selection may be applied to a population of cells comprising MESECs and non-MESEC cells for a selected period of time sufficient to achieve enrichment of the MESECs. The metabolic selection period may be applied for selected time periods such as about 12 hours, about 24 hours, about 36 hours, about 48 hours, about 60 hours, about 72 hours, about four days, about five days, about six days, about seven days, or longer. Generally, a period of about 24-72 hours will substantially deplete non-MESEC cells. For example, in the context of differentiating hepatocytes from pluripotent cells, application of a 1-3 day pulse of glucose, glutamine and pyruvate deprivation to mixed populations comprising hepatocytes and non-hepatocytes suchs as nonundifferentiated hPSCs, day 6 liver bud progenitors, day 6 midgut/hindgut endoderm cells will be sufficient to deplete a large portion or substantially all of the non-hepatocytes.

In the general method of the invention, the metabolic selection is applied to a mixed population of cultured cells comprising both MESECs and non-MESECs. By the metabolic selection process, a substantial portion of the non-MESEC cells die, while a substantial portion of MESEC cells surive, such that the resulting population of cells is enriched in MESEC cells, in comparison to the starting population prior to metabolic selection. In various embodiments, the proportion of MESEC cells in the post-selection population of cells comprises at least 20%, at least 30%, at least 40%, at least 50%, at least 60%, at least 70%, at least 75%, at least 80%, at least 85%, at least 90%, at least 95%, or at least 99%. In one embodiment, the MESECs comprise hepatocytes, for example, hepatocyte-like cells, for example, hepatocytes or hepatocyte-like cells derived from pluripotent cells or other cell types. In some embodiments, the hepatocytes or hepatocyte-like cells are present in a post-selection population comprising, for example at a proportion of at least 70%, at least 75%, at least 80%, at least 85%, at least 90%, at least 95%, or at least 99% purity, or a substantially pure population of hepatocytes or hepatocyte-like cells. In some embodiments the MESECs are smooth muscle cells or astrocytes.

In some implementations, following the application of metabolic selection to deplete non-MESECs and to enrich the population in MESECs, such as hepatocytes, the population of cells is then cultured in a recovery medium comprising glucose, glucose and glutamine, or glucose, or glucose, glutamine, and pyruvate. For example, in one embodiment, the recovery medium comprisies CDM4B media, for example, CDM4B medium supplemented with Forskolin (10 μM), Dexamethasone (10 μM), RO4929097 (2 μM), AA2P (200 μg/mL), and Insulin (10 μg/mL), DMEM, F12, or IMDM. The cells may be cultured in the recovery medium for any selected period of time, for example, 12 hours, 24 hours, 36 hours, or longer. Following the culture period in recovery medium, the cells may be dissociated, frozen, or utilized in therapeutic or experimental protocols.

Optionally, a viable cell sorting process may be applied to the population of cultured cells following metabolic selection. Any methodology for separation of viable cells from a population of viable and non-viable cells may be applied.

Differentiated MESECs From Pluripotent Cells. The metabolic selection methods of the invention may be applied to any population of cells to enrich the population in MESECs, for example, hepatocytes. In a primary implementation, the metabolic selection methods of the invention are applied for the enrichment of MESECs that have been produced in culture from another cell source, for example, differentiated from pluripotent cells or other precursor cells.

In one implementation, the scope of the invention encompasses the application of metabolic selection to a population of hepatocytes, for example, hepatocyte-like cells and/or cells which express one or more of ALB, FGB, SERPINA1, CPS1, ARG1, TTR, FAH, and AAT, wherein the hepatocytes have been differentiated from a population of progenitor cells such as pluripotent cells and are present in a mixed population of hepatocytes and other cell types. By the metabolic selection process, the population of cells may be enriched in hepatocytes, for example to produce populations having a high proportion of hepatocytes, including populations comprising essentially pure populations of hepatocytes.

In one implementation, the mixed population of cells is produced from pluripotent cell sources according to the protocols described in the Examples section hereof. In one embodiment, the mixed population of cells is produced from pluripotent cells according to a process wherein: Pluripotent cells such as hPSCs are differentiated into anteriormost primitive streak cells; anteriormost primitive streak cells are differentiated into definitive endoderm; definitive endoderm cells are differentiated foregut cells, foregut cells are differentiated into early liver progenitor cells, early liver progenitor cells are differentiated into liver bud progenitor cells, liver bud progenitor cells are differentiated into early hepatocytes, early hepatocytes are differentiated to mature hepatocytes, for example hepatocyte-like cells, for example, cells expressing one or more of markers ALB, FGB, SERPINA1, CPS1, ARG1, TTR, FAH, and AAT.

The metabolic selection methods of the invention may be applied to populations of cells comprising hepatocytes, for example, hepatocyte-like cells and/or cells expressing one or more of markers ALB, FGB, SERPINA1, CPS1, ARG1, TTR, FAH, and AAT, wherein the population of cells has been produced by any hepatocyte differentiation protocol known in the art. Exemplary protocols for the production of hepatocytes from pluripotent cells or other cell sources include: Takebe et al., Vascularized and functional human liver from an iPSC-derived organ bud transplant. Nature 499, 481-484 (2013); Ogawa et al., Three-dimensional culture and cAMP signaling promote the maturation of human pluripotent stem cell-derived hepatocytes. Development 140, 3285-3296 (2013); Carpentier et al., Engrafted human stem cell-derived hepatocytes establish an infectious HCV murine model. Journal of Clinical Investigation 124, 4953-4964 (2014); Si Tayeb et al., Highly efficient generation of human hepatocyte-like cells from induced pluripotent stem cells. Hepatology 51, 297-305 (2010); Zhao et al., Promotion of the efficient metabolic maturation of human pluripotent stem cell-derived hepatocytes by correcting specification defects. Cell Research 23, 157-161 (2012); Touboul et al., Generation of functional hepatocytes from human embryonic stem cells under chemically defined conditions that recapitulate liver development. Hepatology 51, 1754-1765 (2010); Sumi, et al., Defining early lineage specification of human embryonic stem cells by the orchestrated balance of canonical Wnt/-catenin, Activin/Nodal and BMP signaling. Development 135, 2969-2979 (2008); Yanagida et al., An In Vitro Expansion System for Generation of Human iPS Cell-Derived Hepatic Progenitor-Like Cells Exhibiting a Bipotent Differentiation Potential. PLoS ONE 8, e67541 (2013); Gadue, et al. Wnt and TGF-β signaling are required for the induction of an in vitro model of primitive streak formation using embryonic stem cells. Proceedings of the National Academy of Sciences 103, 16806-16811 (2006); Gouon-Evans et al., BMP-4 is required for hepatic specification of mouse embryonic stem cell-derived definitive endoderm. Nature Biotechnology 24, 1402-1411 (2006); Ang et al., A Roadmap for Human Liver Differentiation from Pluripotent Stem Cells. Cell Reports 22, 2190-2205 (2018); Cai et al., Directed differentiation of human embryonic stem cells into functional hepatic cells. Hepatology 45, 1229-1239 (2007); Agarwal et al., Efficient Differentiation of Functional Hepatocytes from Human Embryonic Stem Cells. Stem Cells 26, 1117-1127 (2008); Haridass et al., Repopulation Efficiencies of Adult Hepatocytes, Fetal Liver Progenitor Cells, and Embryonic Stem Cell-Derived Hepatic Cells in Albumin-Promoter-Enhancer Urokinase-Type Plasminogen Activator Mice. The American Journal of Pathology 175, 1483-1492 (2009); Han, Generation of Functional Hepatic Cells from Pluripotent Stem Cells. Journal of Stem Cell Research and Therapy 1, 1-7 (2012); Song et al., Efficient generation of hepatocyte-like cells from human induced pluripotent stem cells. Cell Research 19, 1233-1242 (2009); Shiraki et al., Differentiation of mouse and human embryonic stem cells into hepatic lineages. Genes to Cells 13, 731-746 (2008); Avior et al., Microbial-derived lithocholic acid and vitamin K2 drive the metabolic maturation of pluripotent stem cells-derived and fetal hepatocytes. Hepatology 62, 265-278 (2015); Carpentier et al., Hepatic differentiation of human pluripotent stem cells in miniaturized format suitable for high-throughput screen. Stem Cell Research 16, 640-650 (2016); Tolosa et al., Transplantation of hESC-derived hepatocytes protects mice from liver injury. Stem Cell Research & Therapy 6, 246 (2015); Nagamoto et al., Transplantation of a human iPSC-derived hepatocyte sheet increases survival in mice with acute liver failure. Journal of Hepatology 64, 1068-1075 (2016); Ma et al., The nuclear receptor THRB facilitates differentiation of human PSCs into more mature hepatocytes. Cell Stem Cell 29, 795-809 e711 (2022).

Embodiments of the invention include a composition of hepatocytes in selection medium. Emobidments of the invention include a population of cells comprising hepatocytes, for example, hepatocyte-like cells, for example hepatocytes produced from pluripotent cells according to the methods of the invention disclosed herein. In some embodiments, a composition of selected hepatocytes is provided, where the cells have been maintained in culture for at least about 12 hours, at least about 24 hours, at least about 36 hours, at least about 48 hours. In some such embodiments the cell have been genetically modified in culture. In some such embodiments the purified cell population is provided in a pharmaceutically acceptable excipient for transplantation, and may be provided in an effective dose for transplantation, e.g. at least about 10⁵ cells, at least about 10⁶ cells, at least about 10⁷ cells, at least about 10⁸ cells or more.

Aspects of the instant disclosure include method of screening pharmacological agents using hepatocytes derived according to the methods described herein. In some instances, a plurality of cell populations derived according to the methods as described herein are contacted with a plurality of pharmacological agents in order to screen for agents producing a cellular response of interest. A cellular response of interest may be any cellular response including but not limited to, e.g., cell death, cell survival, cell self-renewal, proliferation, differentiation, expression of one or more markers, loss of expression of one or more markers, change in morphology, change in cellular physiology, cellular engraftment, change in cell motility, change in cell migration, production of a particular cellular component, cease of production of a particular cellular component, change in metabolic output, response to stress, and the like.

Screening pharmacological agents using cells described herein may be performed in vitro, e.g., in a tissue culture chamber, on a slide, etc., or may be performed in vivo, e.g., in an animal host, etc. Cells used in such screening assays may be genetically altered or may be unaltered. In some instances, cells generated according to the methods as described herein are used in multiplexed in vitro pharmacological screening. Methods for evaluating cellular responses during in vitro screening are well-known in the art and include but are not limited to, e.g., microscopic methods (e.g., light microscopy, electron microscopy, etc.), expression assays, enzymatic assays, cytological assays (e.g., cellular staining), genomics, transcriptomics, metabolomics, and the like.

In some instances, cells generated according to the methods as described herein are introduced into a host animal and the host animal may be administered a pharmacological agent in order to screen for a response from the introduced cells. In some instances, the cells of the in vivo assay may be directly evaluated, e.g., for an intrinsic response to a pharmacological agent. In some instances, the host animal of the in vivo assay may be evaluated as an indirect measurement of the response of the cells to the pharmacological agent.

In certain embodiments, the subject disclosure includes screening cells derived according to the methods described herein as a method of therapy of an animal model of disease and/or a human disease. Methods of screening cells derived according to the methods described herein as a method of therapy may be, in some instances, performed according to those methods described below regarding using such cells in therapeutic protocols.

In certain embodiments, the subject disclosure includes screening cells derived according to the methods described herein introduced to a host animal as a method of directly evaluating the cells or particular cellular behaviors, e.g., due to an introduced genetic modification or a naturally derived mutation. In one embodiment, genetically modified cells, e.g., having at least one modified genomic locus, derived according to the methods described herein may be introduced into a host animal and the ability of the cells to differentiate into a particular tissue or cell type may be evaluated. In another embodiment, genetically modified cells derived according to the methods described herein may be introduced into a host animal and the behavior of the cells within the host animal and/or within a tissue of the host animal may be evaluated. In another embodiment, cells derived from a donor organism having a particular mutation or phenotype and lineage restricted according to the methods described herein may be introduced into a host animal and the behavior of the cells within the host animal and/or within a tissue of the host animal may be evaluated, including, e.g., the ability of the cells to differentiate into one or more tissue or cell types. The cells may be introduced into the host animal in a autologous graft, an allograft, or a xenograft such that the introduced cells may be derived from the host animal, a separate donor of the same species as the host animal, or a separate donor of a different species as compared to the host animal, respectively.

Aspects of the disclosure include methods for lessening the symptoms of and/or ameliorating a dysfunction in hepatic dysfunction or disorder. Treatment methods described herein include therapeutic treatments, in which the subject is inflicted prior to administration, and prophylactic treatments, in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to hepatic dysfunction or disorder and/or a family history indicating increased risk of hepatic dysfunction or disorder), in which case the treatment can be a prophylactic treatment. In some embodiments, the individual to be treated is an individual with hepatic dysfunction or disorder. Any and all forms of hepatic dysfunction, whether treated or untreated, or resulting from any primary condition, whether treated or untreated, are suitable hepatic dysfunctions or disorders to be treated by the subject methods described herein.

In some instances, the treatment methods described herein include the alleviation or reduction or prevention of one or more symptoms of hepatic dysfunction or disorder. Symptoms of hepatic dysfunction or disorder will vary, may be infrequent, occasional, frequent, or constant. The methods of treatment described herein include administering a therapeutically effective amount of a population, e.g., an essentially homogenous population, of hepatocytes to a subject in need thereof in order to treat the subject for a hepatic dysfunction or deficiency.

The effective amount administered varies depending upon the goal of the administration, the health and physical condition of the individual to be treated, age, the taxonomic group of individual to be treated (e.g., human, non-human primate, primate, etc.), the degree of resolution desired (e.g., the amount of alleviation or reduction of symptoms), the formulation of the cell composition, the treating clinician's assessment of the medical situation, and other relevant factors.

A “therapeutically effective dose” or “therapeutic dose” is an amount sufficient to effect desired clinical results (i.e., achieve therapeutic efficacy) or reduce, alleviate, or prevent symptoms to a desired extent as determined by the patient or the clinician. A therapeutically effective dose can be administered in one or more administrations. For purposes of this disclosure, a therapeutically effective dose of cells and/or compositions is an amount that is sufficient, when administered to (e.g., transplanted into) the individual, to palliate, ameliorate, stabilize, reverse, prevent, slow or delay the progression of the disease state by, for example, inducing stabilization, repair, or regeneration of existing tissues.

In some embodiments, a therapeutically effective dose of cells (e.g., derived hepatic cell types, etc.) is one cell or more (e.g., 1×10² or more, 5×10² or more, 1×10³ or more, 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective dose of cells is in a range of from 1×10³ cells to 1×10¹⁰ cells (e.g., from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁶ cells to 1×10¹⁰ cells, from 5×10⁶ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁹ cells, from 5×10⁵ cells to 5×10⁹ cells, from 1×10⁶ cells to 5×10⁹ cells, from 5×10⁶ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁶ cells to 1×10⁹ cells, from 5×10⁶ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁶ cells to 5×10⁸ cells, from 5×10⁶ cells to 5×10⁸ cells, from 1×10⁷ cells to 5×10⁸ cells, from 5×10⁷ cells to 5×10⁸ cells, or from 1×10⁸ cells to 5×10⁸ cells).

In some embodiments, the concentration of cells (e.g., metabolically selected hepatocytes, etc.) to be administered is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/ml to 1×10⁸ cells/ml, from 1×10⁶ cells/ml to 5×10⁷ cells/ml, from 1×10⁶ cells/ml to 1×10⁷ cells/ml, from 1×10⁶ cells/ml to 6×10⁶ cells/ml, or from 2×10⁶ cells/ml to 8×10⁶ cells/ml).

In some embodiments, the concentration of cells to be administered is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁶ cells/ml or more, 4×10⁶ cells/ml or more, 5×10⁶ cells/ml or more, 6×10⁶ cells/ml or more, 7×10⁶ cells/ml or more, or 8×10⁶ cells/ml or more).

A therapeutically effective dose of cells may be delivered or prepared and any suitable medium, including but not limited to, e.g., those described herein. Suitable medium for the delivery of a therapeutically effective dose of cells will vary and may depend on, e.g., the type of pluripotent cells from which the effective dose of cells is derived or the type of derived cells of the effective dose. In some instances, a suitable medium may be a basal medium. “Cell medium” as used herein are not limited to liquid media may, in some instances, include non-liquid components or combinations of liquid media and non-liquid components. Non-liquid components that may find use a delivery or preparation medium include those described herein and those known in the art. In some instances, non-liquid components include natural or synthetic extra cellular matric components including but not limited to, e.g., basement membrane matrix components and the like.

In some instances, an effective dose of the cells described herein may be co-administered with one or more additional agents (e.g., prepared in a suitable medium). For example, an effective dose of derived hepatocytes from a homogenous population of cells derived according to the methods described herein may be co-administered with one or more additional agents. Additional agents useful in such co-administration include agents that improve the overall effectiveness of the effective dose of cells or decrease the dose of cells necessary to achieve an effect essentially equal to administration of an effective dose of the cells without the additional agent. Non-limiting examples of additional agents that may be co-administered with derived hepatocytes derived according to the methods described herein include: conventional agents for treating diseases of the liver, pro-survival factors, pro-engraftment factors, functional mobilization agents, and the like.

By pro-survival factors is meant a factor or agent that may be added to the medium, culture media, delivery excipient, or storage solution that promotes the survival of a desired cell type. Such pro-survival factors may be general pro-survival factors that generally promote the survival of most cell types or may be specific pro-survival factors that only promote the survival of certain specific cell types. In some instances, pro-survival factors of the subject disclosure include but are not limited to, e.g., Rho-associated kinase (ROCK) inhibitor, pinacidil, allopurinol, uricase, cyclosporine (e.g., low dose, i.e., sub-immunosuppressive dose, cyclosporine), ZVAD-fmk, pro-survival cytokines (e.g., insulin-like growth factor-1 (IGF-1)), extra cellular matrix (ECM) components, hydrogels, matrigel, collagen, gelatin, agarose, alginate, poly(ethylene glycol), hyaluronic acid, etc.

By pro-engraftment factors is meant a factor or agent that may be added to the administered dose or the delivery excipient or the cell storage solution that, upon delivery of the cells into a subject for treatment, increase the engraftment of the administered cells into the tissue targeted for engraftment and therapy. In some instances, pro-engraftment factors include factors that physically retain the administered cells at the delivery site, e.g., the injection site in the case of direct injection to the affected area, including but not limited to, e.g., gels, polymers, and highly viscous liquids that have physical properties that prevent the administered cells from freely diffusing. Such gels, polymers, and highly viscous liquids include but are not limited to e.g., ECM components, hydrogels, matrigel, collagen, gelatin, agarose, alginate, poly(ethylene glycol), and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The cells may be introduced by injection, catheter, intravenous perfusion, or the like. The cells may be frozen at liquid nitrogen temperatures and stored for long periods of time, being capable of use upon thawing. Once thawed, the cells may be expanded by use of growth factors and/or feeder cells or in feeder-free conditions associated with progenitor cell proliferation and differentiation. In some instances, the cells may be administered fresh such that the cells are expanded and differentiated and administer without being frozen.

The cells (e.g., hepatocytes) and/or compositions of this disclosure can be supplied in the form of a pharmaceutical composition, comprising an isotonic excipient or buffer or media prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells may be autologously derived. By autologously derived it is meant that the cells are derived from the subject that is to be treated with the cells. The cells may be derived from a tissue sample obtained from the subject including but not limited to, e.g., a blood sample (e.g., a peripheral blood sample), a skin sample, a bone marrow sample, and the like. In some instances, the sample from which cells are derived may be a biopsy or swab, e.g., a biopsy or swab collected to diagnose, monitor, or otherwise evaluate the subject, e.g., diagnose the subject for a hepatic dysfunction or deficiency. In some instances, the autologous sample from which the cells are derived may be a previously collected and stored sample, e.g., a banked tissue sample, from the subject to be treated. In some instances, cells may be non-autologously derived. By non-autologously derived it is meant that the cells are not derived from the subject that is to be treated with the cells. In some instances, non-autologously derived cells may be xeno-derived (i.e., derived from a non-human animal) or allo-derived (i.e. derived from a human donor other than the subject to be treated). Non-autologously derived cells or tissue may be derived from any convenient source of cells or tissue collected by any convenient means.

Whether to use autologously derived or non-autologously derived cells may be determined according to the discretion of the subject's clinician and may depend on, e.g., the health, age, genetic predisposition or other physical state of the subject. In some instances, autologous cells may be preferred, including, e.g., to decrease the risk or immune rejection of the transplanted cells. In some instances, non-autologous cells may be preferred, including, e.g., when the subject has a genetic defect that affects the liver.

Methods of derivation of pluripotent progenitor cells from an autologous or non-autologous tissue useful in the methods described herein include but are not limited to, e.g., methods of embryonic stem cell derivation and methods of induced pluripotent stem cell derivation. In some instances, methods as described herein may be performed using non-autologous pluripotent progenitor cells previously derived including, e.g., those publicly or available or commercially available (e.g., from Biotime, Inc., Alameda, CA). In some instances, methods as described herein may be performed using newly derived non-autologous pluripotent progenitor cells or newly derived autologous pluripotent progenitor cells including but not limited to, e.g., newly derived embryonic stem cells (ESC) (including, e.g., those derived under xeno-free conditions as described in, e.g., Lei et al. (2007) Cell Research, 17:682-688) and newly derived induced pluripotent stem cells (IPS). General methods of inducing pluripotency to derive pluripotent progenitor cells are described in, e.g., Rodolfa K T, (2008) Inducing pluripotency, StemBook, ed. The Stem Cell Research Community, doi/10.3824/stembook.1.22.1 and Selvaraj et al. (2010) Trends Biotechnol, 28 (4) 214-23, the disclosures of which are incorporated herein by reference. In some instances, pluripotent progenitor cells, e.g., iPS cells, useful in the methods described herein are derived by reprogramming and are genetically unmodified, including e.g., those derived by integration-free reprogramming methods, including but not limited to those described in Goh et al. (2013) PLoS ONE 8 (11): e81622; Awe et al (2013) Stem Cell Research & Therapy, 4:87; Varga (2014) Exp Cell Res, 322 (2) 335-44; Jia et al. (2010) Nat Methods, 7 (3): 197-9; Fusaki et al. (2009) Proc Jpn Acad Ser B Phys Biol Sci. 85 (8): 348-62; Shao & Wu, (2010) Expert Opin Biol Ther. 10 (2): 231-42; the disclosures of which are incorporated herein by reference.

In some instances, the derived or obtained pluripotent progenitor cells are prepared, dissociated, maintained and/or expanded in culture prior to being differentiated and/or lineage restricted as described herein.

In some instances, before differentiation or lineage restriction of the pluripotent progenitor cells the pluripotent progenitor cells are dissociated, e.g., to generate a single-cell suspension. In some instances, the dissociation of the pluripotent progenitors is chemical, molecular (e.g., enzyme mediated), or mechanical dissociation. Methods of chemical, molecular, and/or enzyme mediated dissociation will vary and in some instances may include but are not limited to the use of, e.g., trypsin, TrypLE Express™, TrypLE Select™, Accutase®, StemPro® (Life Technologies, Inc., Grand Island, NY), calcium and magnesium free media, low calcium and magnesium medium, and the like. In some instances the dissociation media may further include pro-survival factors including but not limited to, e.g., Rho-associated kinase (ROCK) inhibitor, pinacidil, allopurinol, uricase, cyclosporine (e.g., low does, i.e., sub-immunosuppressive dose, cyclosporine), ZVAD-fmk, pro-survival cytokines (e.g., insulin-like growth factor-1 (IGF-1)), Thiazovivin, etc.

In some instances, methods of culturing pluripotent stem cells include xeno-free culture conditions wherein, e.g., human cells are not cultured with any reagents derived from non-human animals. In some instances, methods culturing of pluripotent stem cells include feeder-free culture conditions, wherein the pluripotent stem cells are cultured under conditions that do not require feeder cells and/or in feeder cell free medium, including e.g., commercially available feeder-free mediums, such as, e.g., those available from STEMCELL Technologies, Inc. (Vancouver, BC). In some instances, methods culturing of pluripotent stem cells include culture conditions that include supplemental serum, including e.g. supplement of autologously derived serum, e.g., as described in Stute et al. (2004) Exp Hematol, 32 (12): 1212-25. In some instances, methods of culturing of pluripotent cells or derivatives thereof include culture conditions that are serum-free, meaning the culture media does not contain animal, mammal, or human derived serum. Serum-free culture conditions may be performed for only a portion of the life of the culture or may performed for the entire life of the culture. In some instances, serum-free culture conditions are used for a particular method step or procedure, e.g., during differentiation, during lineage restriction, prior to or during harvesting, etc. As is known in the art, in some instances, cells may be cultured in two dimensional or three dimensional formats (e.g., on non-coated or coated surfaces or within a solid or semi-solid matrix). Instances where two dimensional or three dimensional culture is appropriate for use in the methods as described herein, e.g., to promote survival or differentiation of a desired cell type, will be readily apparent to the ordinary skilled artisan. In some instance the pluripotent progenitor cell media includes one or more pro-survival factors, e.g., including those described herein. General methods of culturing human pluripotent progenitor cells are described in, e.g., Freshney et al. (2007) Culture of human stem cells, Wiley-Interscience, Hoboken, NJ and Borowski et al. (2012) Basic pluripotent stem cell culture protocols, StemBook, ed. The Stem Cell Research Community, the disclosures of which are incorporated herein by reference.

Pluripotent cells develop into hepatocytes through multiple consecutive branching lineage choices. Liver development has been reconstituted through a sequence of six consecutive lineage choices. Multiple developmental signals (e.g., retinoid, TGF-β, Wnt, Hedgehog, BMP, and other signals) have opposing effects within 24 hr, initially specifying one fate and then subsequently repressing its formation. The temporally dynamic action of these signals contrasts with how these signals are typically added for multiple days in some prevailing differentiation schema. Manipulating signals in a temporally dynamic fashion enabled the faster production of liver bud progenitors from hPSCs within 6 days. The hPSC-derived liver bud progenitors produced could further differentiate into FAH⁺ hepatocyte-like cells that function in vitro and improve short-term survival in a model of liver injury. Liver bud progenitors are differentiated into ALBUMIN hepatocyte-like cells by inhibition of NOTCH and TGF-β. Methods for this process are disclosed in Ang et al. (2018), herein specifically incorporated by reference. High insulin levels, together with a stabilized ascorbic acid derivative (ascorbic acid-2-phosphate [AAP]) greatly promoted the expression of tyrosine metabolic pathway genes PAH, HGD, HPD, TAT, MAI, and FAH and other liver markers by 18 days of differentiation. PKA agonists (e.g., 8-bromo-cAMP and forskolin) also had a similar effect. Combining these signals efficiently generated ALBUMIN⁺ hepatocyte-like cells by day 18 of differentiation, which displayed various hepatocyte functions in vitro.

In some instances, the pluripotent progenitor cells used according to the methods described herein may be genetically unmodified. By “genetically unmodified” is meant that essentially no modification of the genome of the cells transplanted into the subject has been performed. Encompassed within the term genetically unmodified are instances wherein transient genetic modification is performed at some point during the derivation of the cells but essentially no genetic modification persists in the cells that are eventually transplanted into the subject (i.e. the cells are essentially indistinguishable before the transient genetic modification and after the course of the transient modification). Also encompassed within the term genetically unmodified are instances wherein the genome of the cells is not transiently or stably modified, e.g., where the cells are manipulated, e.g., pluripotent progenitors are derived or cells are transformed, without genetic modification (e.g., modification of the nucleotide sequence of the genome) of the cells.

In some instances, the cells used according to the methods described herein may be genetically modified. By “genetically modified” is meant that at least one nucleotide is added to, changed within, or deleted from of the genome of the cell. In some instances, the genetic modification may be an insertion of a heterologous sequence, e.g., a sequence that encodes a tag, a label sequence, a reporter, a selectable marker, a gene encoding a protein from a species different from that of the host cell, etc. In some instances, the genetic modification corrects a defect or a mutation within the cell, e.g., corrects an anomalous mutation that confers a hepaticly derived tissue dysfunction or deficiency. In some instances, the genetic modification deletes or renders inoperable an endogenous gene of the host cell. In some instances, the genetic modification enhances an endogenous gene of the host cell. In some instances, the genetic modification represents a change that enhances survival, control of proliferation, and the like. Cells may be genetically altered by transfection or transduction with a suitable vector, homologous recombination, or other appropriate technique, so that they express a heterologous sequence or have altered expression of an endogenous gene.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### Systems

Also provided are systems for use in practicing the subject methods. Systems of the subject disclosure may include a cell production system, e.g., for the production of a homogenous or highly pure population of purified hepatocytes from pluripotent progenitor cells.

In some instances, the cell production system includes a cell culture chamber or cell culture vessel for the culture of desired cell types. Such cell culture chambers may be configured for the expansion of pluripotent progenitor cells and for the differentiation and/or lineage restriction of such pluripotent progenitor cells into desired cell types, e.g., derived hepatocytes and/or differentiated hepatic cell types. In some instances, the cell culture chamber is also configured for the expansion of hepatocytes and/or differentiated hepatic cell types. In certain embodiments, the cell culture chamber or cell culture vessel may be an open culture system, including but not limited to e.g., tissue culture dishes, tissue culture plates, tissue culture multi-well plates, tissue culture flasks, etc. In certain embodiments, the cell culture chamber or cell culture vessel may be a closed culture system, including e.g., a bioreactor, a stacked tissue culture vessel (e.g., CellSTACK Culture Chambers available from Corning, Inc. Corning, NY). In some instances, culture media and or other factors or agents may be exchanged in and out of the cell culture chamber through the use of one or more pumps (e.g., syringe pumps, peristaltic pumps, etc.) or gravity flow devices. In instances where the cells are cultured under sterile conditions the culture system may allow for the sterile exchange of culture media, e.g., through the use of sterile tubing connected, sealed, and reconnected through the use of a sterile devices, including but not limited to, e.g., a sterile tube welder and/or a sterile tube sealer. The cell culture system may be configured to control certain environmental conditions, including but not limited to e.g., temperature, humidity, light exposure, air composition (e.g., oxygen levels, carbon dioxide levels, etc.) to achieve the conditions necessary for expansion and/or differentiation of desired cell types. In some instances, the cell culture chamber may include a cell culture vessel that includes one or more patterned cell culture substrates or one or more arrays of patterned cell culture substrates as described herein.

The cell culture chamber may be configured for the production of cells for clinical use, e.g., according to current good manufacturing practice (CGMP) compliant cell culture practices, including the methods and configurations described in e.g., Fekete et al. PLoS ONE (2012) 7 (8): e43255; Pham et al. (2014) J Trans Med 12:56; Gastens et al. (2007) Cell Transplant 16 (7): 685-96; Fernandes et al. (2013) Stem Cell Bioprocessing: For Cellular Therapy, Diagnostics and Drug Development, Burlington, Oxford: Elsevier Science: Woodhead Publishing, the disclosures of which are incorporated herein by reference.

The cell production system may, in some instances, by computer controlled and/or automated. Automated and/or computer controlled cell production systems may include a “memory” that is capable of storing information such that it is accessible and retrievable at a later time or date by a computer. Any convenient data storage structure may be chosen, based on the means used to access the stored information. In certain aspects, the information may be stored in a “permanent memory” (i.e. memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

In in certain instances, a computer controlled and/or automated cell culture system may include a module or program stored in memory for production of cells according to the methods described herein. Such a module may include instructions for the administration of induction agent and/or induction compositions, e.g., at particular timing intervals or according to a particular schedule, in order to generate a desired cell type. In some instances, such a computer module may further include additional modules for routine cell culture tasks including but not limited to, e.g., monitoring and record keeping, media changes, environmental monitoring, etc.

Systems of the present disclosure include components and/or devices for delivering cells produced according to the methods described herein to a subject in need thereof. For example, in some instances a system for treating a subject with a hepatic derived tissue dysfunction or deficiency includes a cell injection system for delivering cells in a carrier, with or without optional adjuvants, to a desired injection site, including diseased tissue, adjacent to diseased tissue, and/or within, on or near a dysfunctioning organ. Such systems utilize known injection devices (e.g., including but not limited to needles, bent needles, cannulas, syringes, pumps, infusion devices, diffusion devices, etc.) and techniques (e.g., including but not limited to intramuscular injection, subcutaneous injection, device-guided injection, etc.). In some instances, a device or technique used for the delivery of a cell scaffold or other bioengineered device may be configured or adapted for use in a cell delivery system for use in delivering cells derived according to the methods described herein

In addition to the above described components systems of the subject disclosure may include a number of additional components, such as data output devices, e.g., monitors and/or speakers, data input devices, e.g., interface ports, keyboards, etc., fluid handling components, power sources, controllers, etc.

### Compositions and Kits

Also provided are compositions and kits for use in the subject methods. The subject compositions and kits include any combination of components for performing the subject methods. In some embodiments, a composition can include, but is not limited to and does not require, the following: cell dissociation agents and/or media, cell reprogramming agents and/or media, pluripotent progenitor cells, cell culture agents and/or media, cell differentiation agents and/or media; conventional agents for treating diseases and/or dysfunctions of the liver, pro-survival factors, pro-engraftment factors, functional mobilization agents and any combination thereof.

In some embodiments, a kit can include, but is not limited to and does not require, the following: any of the above described composition components, a sample collection container, a sample collection device (e.g., a sample collection container that includes a sample enrichment mechanism including, e.g., a filter), a tissue collection device (e.g., a biopsy device), a tissue dissociation device, a cell culture vessel, a cell production system; and any combination thereof.

In some embodiments, a kit can include, but is not limited to and does not require, a cell delivery system and/or a cell injection system configured for delivery of cells derived according to the methods described herein.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is electronic, e.g., a website address which may be used via the internet to access the information at a removed site.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Metabolically purified human stem cell-derived hepatocytes reveal distinct effects of Ebola and Lassa viruses

Ebola and Lassa viruses require biosafety-level-4 (BSL4) containment, infect the liver, and cause deadly hemorrhagic fevers. The cellular effects of these viruses, and whether different families of hemorrhagic-fever viruses elicit similar effects, remain fundamental questions in BSL4 virology. Here we introduce a new approach to create nearly-pure hepatocytes from human pluripotent stem cells: we killed non-liver cells by withholding essential nutrients. Unexpectedly, Ebola and Lassa exerted starkly different effects on human hepatocytes. Ebola infection activated the integrated stress response (ISR) and WNT pathways in hepatocytes in vitro and killed them, whereas Lassa did not. Within non-human primates, Ebola likewise infected hepatocytes and activated ISR signaling in vivo. In summary, we present a single-cell transcriptional and chromatin accessibility roadmap of hepatocyte differentiation, purification, and viral infection.

Risk Group 4 viruses are among the deadliest viruses known on Earth and must be studied in specialized biosafety level 4 (BSL4) laboratories, few of which exist worldwide. Sparingly few approved therapies exist for the devastating diseases caused by these viruses. Accordingly, many of these pathogens have been designated as World Health Organization Priority Diseases, necessitating urgent research and development. Filoviruses such as Ebola virus (~44% fatality rate, species Orthoebolavirus zairense), Sudan virus (~54% fatality rate, species Orthoebolavirus sudanense), and Marburg virus (~81% fatality rate, species Orthomarburgvirus marburgense) cause periodic outbreaks in Africa. These include the 2013-2016 Ebola virus epidemic that killed over 11,000 individuals in West Africa, the 2022 Sudan virus epidemic in Uganda, and the 2023 Marburg virus outbreaks in Equatorial Guinea and Tanzania. Lassa virus (~1% fatality rate, species Lassa mammarenavirus) is an arenavirus that infects up to 500,000 individuals every year in Africa. One important question is what are the effects of these Risk Group 4 viruses on physiologically-relevant human cell-types. Additionally, few, if any, studies have directly compared the effects of different Risk Group 4 viral families—such as filoviruses (family Filoviridae) vs. arenaviruses (family Arenaviridae)—on human cells, in the same experimental system. While many Risk Group 4 viruses cause deadly hemorrhagic fevers in humans, an important and unresolved question is whether distinct families of Risk Group 4 viruses cause common or diverging effects on their shared human cells.

The liver is a primary target of many deadly Risk Group 4 viruses: Ebola, Sudan, Marburg, and Lassa viruses infect liver cells in vivo and can ultimately cause liver damage. Although it has been known for more than 30 years from patient autopsies that Ebola, Sudan, Marburg, and Lassa viruses infect liver cells, the unique constraints of BSL4 experimentation have made it challenging to study the mechanistic effects of these viruses on specific human cell-types, such as hepatocytes. As surrogates, liver cancer cell lines HepG2 and HuH7 have been extensively employed in BSL4 virology to study cellular responses to viral infection and to conduct chemical and genetic screens. However, liver cancer cell lines lack fundamental liver hallmarks, harbor chromosome abnormalities, and are largely incapable of producing interferons, key antiviral cytokines. Alternatively, primary hepatocytes isolated from human beings are the current gold standard for studying liver biology in vitro, but they are scarce, expensive, and vary between individuals. Importantly, it is well-established that primary hepatocytes swiftly lose hepatocyte identity and functions ex vivo.

The ability to generate massive numbers of human hepatocytes would provide a valuable platform to study the effects of Risk Group 4 viruses on a physiologically-relevant cell-type in vitro. Human pluripotent stem cells (hPSCs, including embryonic and induced pluripotent stem cells) can be grown in large numbers and can theoretically generate any cell-type within the body, including hepatocytes. However, hPSCs can generate hundreds of different cell-types, and it remains challenging to differentiate them exclusively into hepatocytes. Despite considerable progress, hPSC differentiation yields heterogeneous cell populations containing hepatocytes commingled with non-liver cells. These contaminating non-liver cells pose challenges for virology, regenerative medicine, and other applications. For instance, one study differentiated hPSCs into a heterogeneous population comprising ~25% albumin⁺ hepatocytes and ~75% unidentified cells to study Ebola virus infection. However, the predominance of non-liver cells made it unclear whether Ebola directly infected hepatocytes or whether any observed cellular responses to Ebola were attributable to hepatocytes and/or the contaminating non-liver cells.

Here we develop “metabolic selection”, a new, rapid, and simple approach to purify hPSC-derived hepatocytes by selectively depleting non-liver cells. This is rooted in the emerging concept that different cell-types have distinct metabolic requirements to survive and can thus be selectively killed by withholding specific nutrients. We hypothesized that hepatocytes could uniquely withstand deprivation of glucose and other specific nutrients, owing to their ability to break down glycogen into glucose and to exploit alternate nutrient sources for energy (gluconeogenesis). Indeed, we found that withholding 3 essential nutrients (glucose, pyruvate, and glutamine) for 1-3 days destroyed non-liver cells in vitro, while hepatocytes survived. Metabolic selection thus produces pure populations of hPSC-derived hepatocytes without recourse to surface marker-based cell sorting or other purification schemas. Critically, metabolic selection purifies hepatocytes based on their metabolic functionality, instead of surface marker expression.

hPSC-derived hepatocytes purified by metabolic selection offer key advantages for BSL4 virology relative to extant liver cancer cell lines. For instance, hPSC-derived hepatocytes behaved similarly to adult primary human hepatocytes in their ability to trigger interferon signaling, whereas liver cancer cell lines could not. Additionally, hPSC-derived hepatocytes showed greater transcriptional similarity to primary hepatocytes than liver cancer cell lines.

Having established highly pure hPSC-derived hepatocytes as an enhanced model system, we discovered that Ebola and Lassa viruses—although both causing viral hemorrhagic fevers in vivo—had starkly different transcriptional and cytopathic effects on human hepatocytes. Notably, Ebola killed hepatocytes, induced WNT and integrated stress response (ISR) pathway genes, and suppressed liver function genes. In contrast, Lassa virus resulted in transient transcriptional changes in genes largely different from those induced by Ebola virus, and did not induce either WNT or ISR pathways. To our knowledge, this is the first direct comparison of how Ebola and Lassa viruses affect human cells in the same experimental system, thus representing a step forward for comparative virology. We further confirmed that Ebola virus directly infects hepatocytes in vivo and activates ISR signaling within a non-human primate model of Ebola virus disease. Finally, we also present a single-cell transcriptional and chromatin accessibility roadmap of hPSC differentiation into hepatocytes, and how they compare with HepG2 and HuH7 liver cancer cell lines. Taken together, the ability to create purified populations of hPSC-derived hepatocytes by metabolic selection will enable a range of applications in BSL4 virology, stem cell research, and regenerative medicine.

### Results:

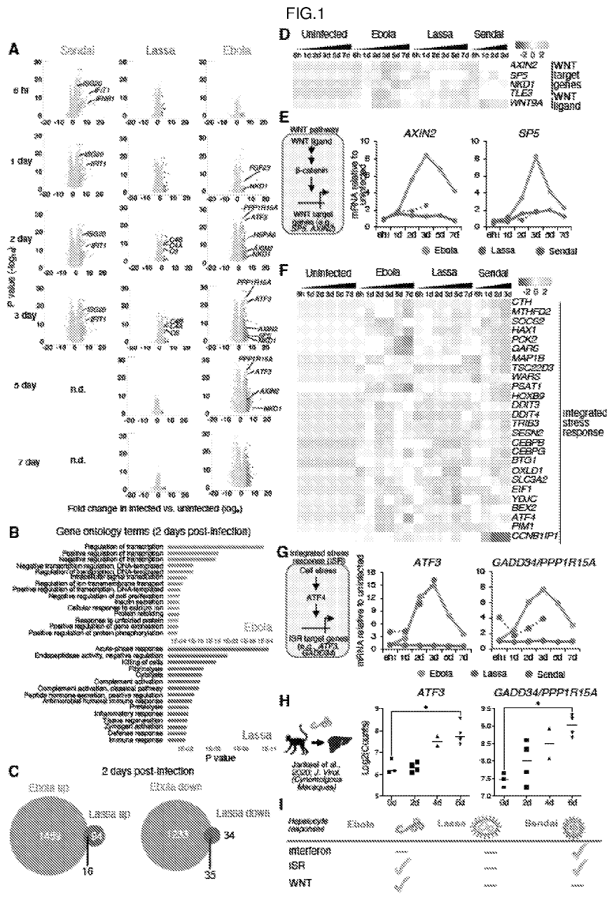

Stepwise changes in gene expression, chromatin accessibility, and cellular diversity during hPSC differentiation into hepatocytes. To chart a stepwise molecular roadmap for hepatocyte development, we applied single-cell RNA-sequencing (scRNAseq), bulk-population RNA-seq, and OmniATAC-seq to a modified version of our previously-developed schema wherein hPSCs are efficiently differentiated into day 1 primitive streak, day 2 definitive endoderm, day 3 posterior foregut, day 6 liver bud progenitors, day 12 early hepatocytes and day 18 hepatocytes (FIGS. 1A-B). At each differentiation step, we provided extracellular signals to direct hPSC differentiation into a desired cell-type while inhibiting other extracellular signals to suppress the formation of unwanted fates (FIG. 1A). Flow cytometry confirmed that our differentiation strategy sequentially generated 99.2% pure MIXL1⁺ primitive streak, 99.8% pure CXCR4⁺ definitive endoderm, and 82.4% pure albumin⁺ hepatocytes by days 1, 2 and 18 of differentiation, respectively (FIG. 1C).

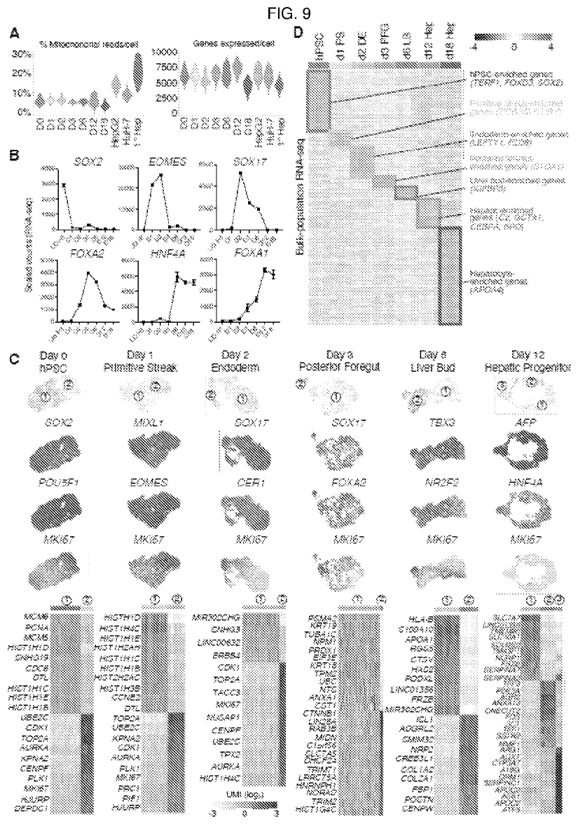

To rigorously assess the purity and synchrony of differentiation, we performed scRNAseq to systematically identify all cell-types arising at each differentiation step (FIG. 1B, FIG. 9A). Within 1 day of hPSC differentiation, pluripotency transcription factor (TF) SOX2 was sharply downregulated, and primitive streak TFs (BRACHYURY, MIXL1) were uniformly expressed (FIGS. 1D, FIG. 9B-C). Next, both day 2 definitive endoderm and day 3 posterior foregut homogeneously expressed the endodermal TF SOX17, with definitive endoderm-specific markers (FZD8, GSC, CER1) expressed in day 2 endoderm but declining in day 3 foregut (FIG. 1D, FIG. 9C). After 6 days of differentiation, the liver bud marker TBX3 was homogeneously expressed, without the apparent expression of intestinal or pancreatic markers (CDX2, PDX1), confirming high differentiation precision (FIG. 1D, FIG. 9C). Sub-clustering analyses suggested that day 1 primitive streak, day 2 definitive endoderm, day 3 posterior foregut, and day 6 liver bud progenitors were largely homogeneous, with residual population heterogeneity at each respective step of differentiation primarily driven by cell-cycle markers (FIG. 9C). This thus reveals largely uniform progression from pluripotency to primitive streak, definitive endoderm, posterior foregut, and liver bud progenitors. On days 12 and 18 of differentiation, liver bud progenitor marker TBX3 gradually decreased, and conversely, hepatocyte markers (ALB, CPS1, APOA4, APOC3) were upregulated (FIG. 1E). These stepwise changes in gene expression were also observed by bulk-population RNA-seq (FIG. 9D).

At each stage of differentiation, there were stepwise changes in chromatin accessibility, with sequential opening and closure of pluripotency, primitive streak, endodermal, and liver gene loci, often coincident with changes in gene expression (FIGS. 1E-F, 9D). Analysis of TF motifs enriched in cell-type-enriched accessible chromatin regions revealed sequential progression from pluripotency (POU5F1) to primitive streak (EOMES), endodermal (SOX17), and eventually liver (HNF4A) TFs, corresponding to the sequential expression of their cognate TFs (FIGS. 1G, 9B). Taken together, this provides a rich resource to potentially discover new markers and regulators of human liver differentiation and can be accessed via an interactive browser.

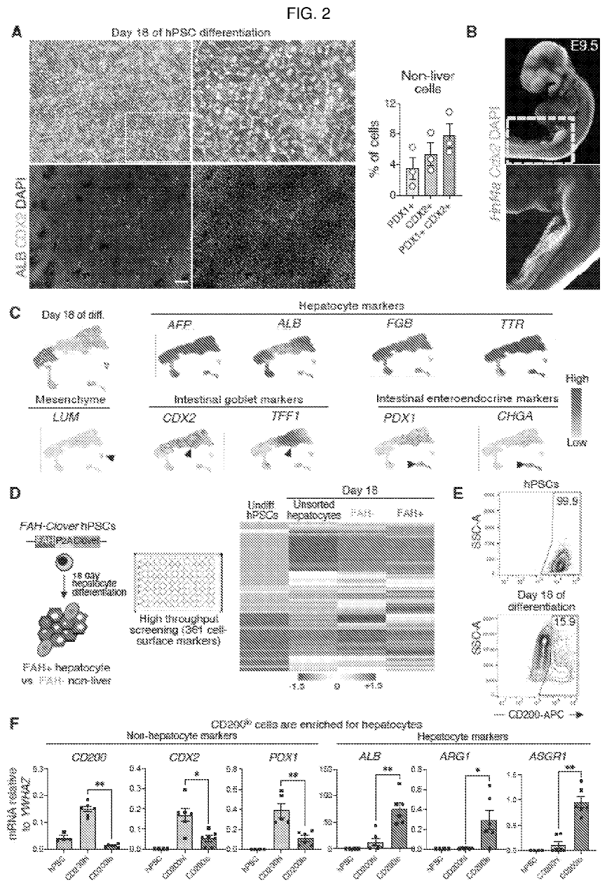

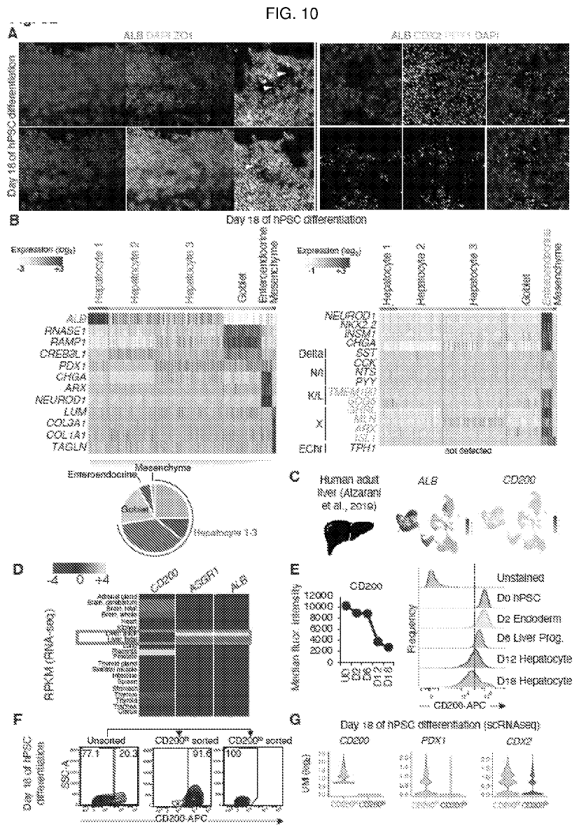

hPSC-derived hepatocytes arise alongside a subset of intestinal cells. Having generated >80% pure albumin⁺ hPSC-derived hepatocytes within 18 days of differentiation, we next explored the identity of the remaining non-liver cells (FIG. 2A, FIG. 10A). We posited that non-liver cells arising during hepatocyte differentiation in vitro might correspond to other endodermal tissues arising near the liver in vivo (e.g., intestines). Indeed, ~8% of cells co-expressed CDX2 and PDX1 (FIG. 2A, FIG. 10A), indicative of the duodenum, which is the anterior region of the small intestine and is adjacent to the developing liver in vivo (FIG. 2B). scRNAseq identified the majority of day 18 cells as ALBUMIN+ hepatocytes but also discovered three smaller subsets of non-liver cells: 1) intestinal goblet-like cells (TFF1, TFF3, CREB3L1+), 2) intestinal enteroendocrine-like cells (CHGA, ARX, NEUROD1+) and 3) trace numbers of mesenchymal cells (LUM, COL3A1+) (FIG. 2C, FIG. 10B). Notably, both intestinal goblet and enteroendocrine cells are specified by NOTCH blockade, consistent with our use of NOTCH inhibitor to differentiate hPSC-derived liver bud progenitors into hepatocytes. In summary, our differentiation approach generates an enriched population of hepatocytes, with some non-liver cells arising contemporaneously. These non-liver cells could interfere with studies of hepatocyte biology and viral infection, and we thus sought to eliminate them.

Cell-surface marker CD200 distinguishes hepatocytes vs. non-liver cells. To eliminate non-liver cells, we first discovered surface markers that distinguish hepatocytes from non-liver cells. To this end, we performed a high-throughput antibody screen of 361 cell-surface markers in FAH-2A-Clover knock-in reporter hPSCs differentiated towards hepatocytes (FIG. 2D). FAH is a tyrosine metabolic enzyme expressed in hepatocytes. We found that CD200 was low in FAH+ hepatocytes but was enriched in FAH-non-liver cells in vitro (FIG. 2E). Likewise, CD200 is expressed in 19 different human tissues but is absent from the liver and hepatocytes (FIGS. 10C-D), reaffirming that CD200 is a non-liver marker in vivo. In vitro, CD200 was highly expressed by hPSCs, definitive endoderm, and liver bud progenitors but significantly decreased by day 18, where only ~15.9% of cells remained CD200^{hi} (FIGS. 2E, 10E). We further tested the value of CD200 as a marker for hepatocyte purification by performing fluorescence-activated cell sorting (FACS) of CD200^{hi} vs. CD2001 cells after 18 days of hPSC differentiation (FIG. 2F, FIG. 10F). CD2001^{lo} cells were enriched for ALB+, ARG1+, and ASGR1+ hepatocytes, whereas CD200^{hi} cells comprised CDX2+ and PDX1+ non-liver cells (FIG. 2F), as confirmed by scRNAseq (FIG. 10G).

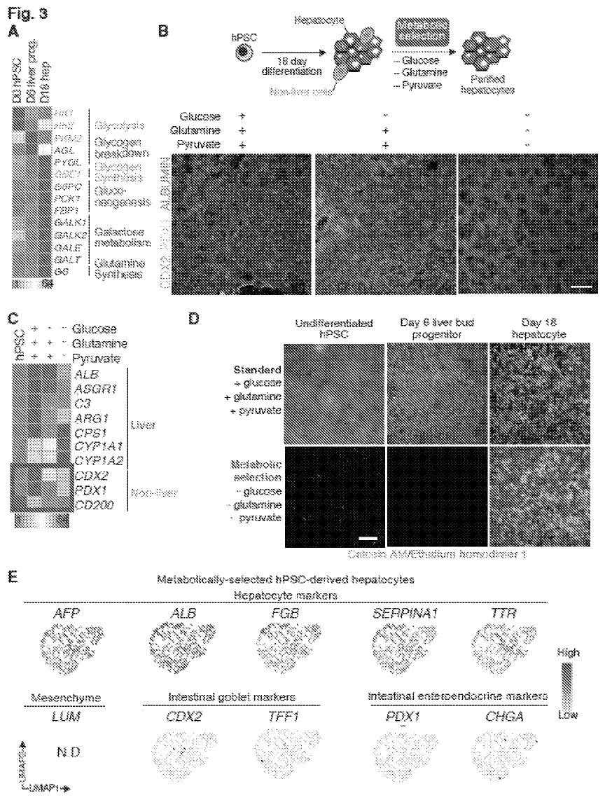

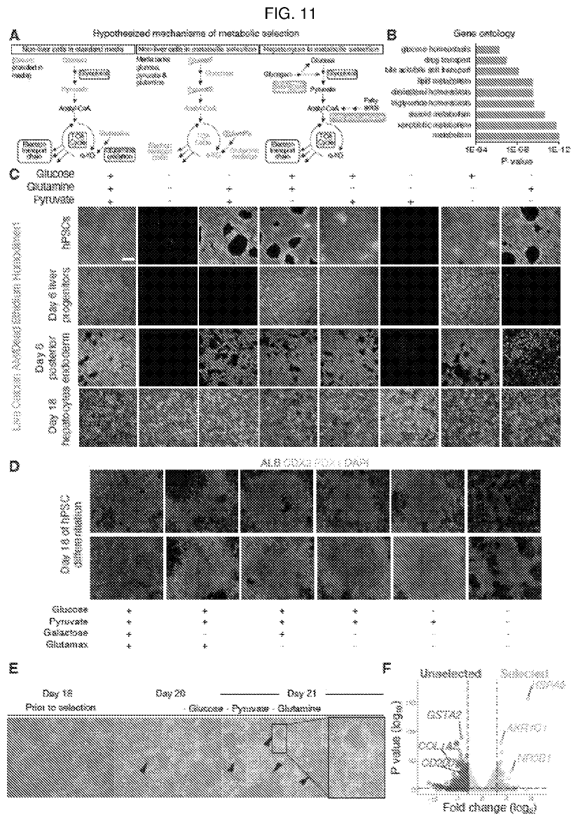

Metabolic selection purifies hPSC-derived hepatocytes and destroys non-liver cells. As a parallel approach to purifying hPSC-derived hepatocytes, we developed a new “metabolic selection” strategy to selectively kill non-liver cells. Remarkably, hepatocytes are one of the few cell types capable of generating glucose de novo and might thus withstand nutrient depletion. We hypothesized that withholding glucose and other nutrients in vitro would eliminate most cell-types (FIG. 11A). Lending confidence that hepatocytes might endure nutrient starvation, we found that hPSC-derived hepatocytes expressed genes involved in energy generation from alternative sources, including genes integral to glycogen breakdown (PGYL, AGL), glycogen synthesis (GBE1), glutamine synthesis (GS), galactose metabolism (GALK1, GALK2, GALE, GALT), and gluconeogenesis (G6PC, FBP1, PCK1) (FIG. 3A). In contrast, hPSC-derived hepatocytes expressed lower levels of glycolysis genes (HK1/2, PKM1/2) than hPSCs (FIG. 3A), suggesting that hepatocytes may be less dependent on glycolysis for energy production. Consistent with this, gene ontology (GO) analysis showed that hPSC-derived hepatocytes expressed a wide range of metabolic genes involved in glucose, lipid, and triglyceride metabolism (FIG. 11B), suggesting that they could exploit alternative substrates such as lipids and triglycerides to survive a starvation episode.

We found that withholding exogenous glucose, glutamine, and pyruvate for 1-3 days led to the enrichment of hPSC-derived hepatocytes and significantly depleted CDX2+ and PDX1+ non-liver cells (FIGS. 3B-C, FIGS. 11C-E). This process of nutrient deprivation—which we refer to as “metabolic selection”—also destroyed undifferentiated hPSCs, day 6 liver bud progenitors, and day 6 midgut/hindgut endoderm (intestinal progenitors), while sparing day 18 hepatocytes (FIG. 3D, FIG. 11C). Combinatorial withdrawal of exogenous nutrients was critical: withholding glucose alone spared undifferentiated hPSCs and failed to robustly eliminate CDX2+ and PDX1+ non-liver cells (FIG. 3B, FIG. 11D). Consistent with how hPSCs can redundantly rely on glycolysis and glutamine oxidation for survival, we found that removing both glucose and glutamine destroyed a range of non-liver cells (FIG. 11C). However, withholding glucose, glutamine, and pyruvate most effectively eliminated non-liver cells, including hPSCs, liver bud progenitors, and midgut/hindgut cells, while sparing hPSC-derived hepatocytes (FIG. 11C). We term our metabolic selection media lacking all three essential nutrients (glucose, glutamine, and pyruvate) as “HepSelect” media.

scRNAseq revealed that two days of HepSelect treatment stringently purified hepatocytes. Hepatocyte markers ALB, AFP, FGB, SERPINA1, and TTR were homogeneously expressed after metabolic selection (FIG. 3E). By contrast, markers of intestinal (CDX2, TFF1, PDX1, CHGA) and mesenchymal cells (LUM) were essentially undetectable (FIG. 3E). RNA-seq and qPCR also confirmed that metabolic selection reduced the expression of non-liver markers including CDX2, PDX1, and CD200 (FIG. 3C, FIG. 11F). In sum, metabolic selection provides a new, effective, and scalable approach to purify hPSC-derived hepatocytes based on their metabolic functionality. Metabolic selection does not require specialized equipment; it solely entails treatment with nutrient-depleted media and is therefore potentially simpler and less expensive to apply on a large scale than other cell purification schemas.

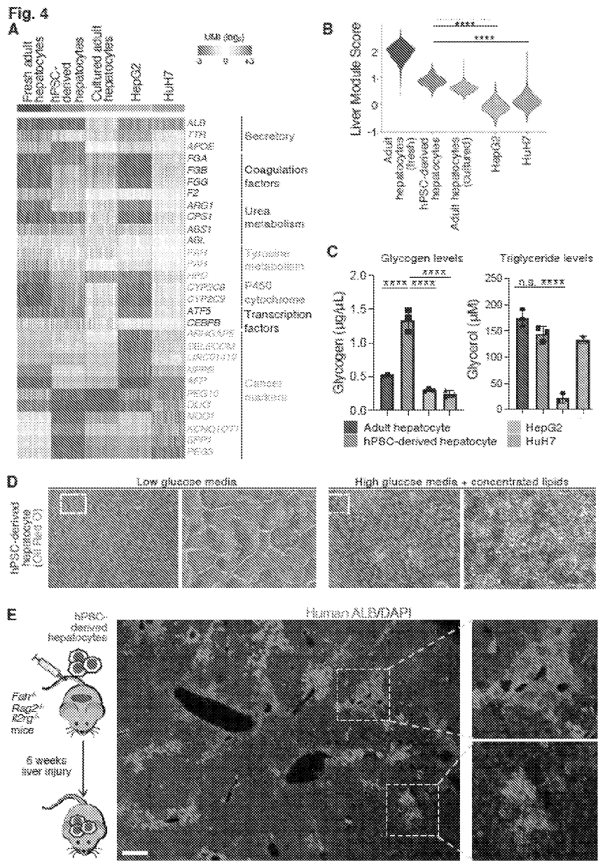

hPSC-derived hepatocytes are transcriptionally more similar to adult hepatocytes than liver cancer cell lines often used in BSL4 virology. scRNAseq revealed that hPSC-derived hepatocytes were more transcriptionally similar to adult hepatocytes than liver cancer cell lines HuH7 and HepG2 (FIGS. 4A-B), which are widely used in BSL4 virology. As controls, we used primary adult human hepatocytes (pooled from 100 individuals to reduce donor-to-donor variability) that were freshly isolated or cultured for 6 days.

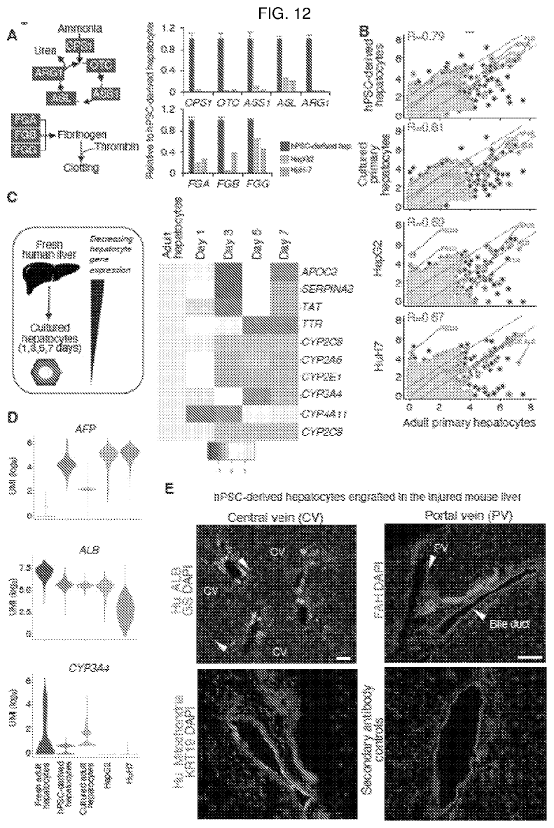

Many hepatocyte genes essential for carrier protein and coagulation factor production, as well as urea, tyrosine, and xenobiotic metabolism, were highly expressed by both hPSC-derived hepatocytes and adult hepatocytes (FIG. 4A). However, these genes showed low or near-absent expression in HuH7 and HepG2 cells (FIG. 4A). Focusing on genes integral to two hallmark hepatocyte functions-detoxification of ammonia into urea (CPS1, OTC, ASS1, ASL, ARG1) and fibrinogen production (FGA, FGB, FGG)—we found that hPSC-derived hepatocytes expressed considerably higher levels of these genes than HuH7 or HepG2 cells (FIG. 4A, FIG. 12A). On the contrary, HuH7 and HepG2 expressed cancer markers PEG3 and PEG10, which were otherwise absent from adult or hPSC-derived hepatocytes (FIG. 4A). Scoring these various liver cell models using prevailing hepatocyte marker signatures showed that metabolically-selected hPSC-derived hepatocytes scored similarly to cultured adult hepatocytes and scored higher than HuH7 and HepG2 (FIG. 4B). Transcriptome-wide analysis revealed that primary adult hepatocytes exhibited 79% and 81% transcriptional similarity to hPSC-derived hepatocytes and cultured adult hepatocytes, respectively (FIG. 12B). On the other hand, HuH7 and HepG2 cancer cells were transcriptionally less similar (67% and 69%, respectively) to adult hepatocytes (FIG. 12B). Taken together, hPSC-derived hepatocytes harbor transcriptional hallmarks of hepatocyte identity and function that have been lost in liver cancer cell lines.

However, hepatocytes in vivo and in vitro were not identical, and exhibited important differences. Upon culture, adult hepatocytes downregulated expression of liver markers and cytochrome enzymes CYP2C8, CYP3A4, TAT, SERPINA3, APOC3, and CYP4A1 (FIG. 12C), and upregulated fetal hepatocyte marker AFP (FIG. 12D). Likewise, hPSC-derived hepatocytes in vitro also expressed AFP but lower CYP3A4 levels than adult hepatocytes (FIG. 12D). In sum, hPSC-derived hepatocytes in vitro are more similar to primary hepatocytes relative to liver cancer cell lines, although they are not identical to primary hepatocytes in vivo.

hPSC-derived hepatocytes display metabolic functions in vitro and engraft the mouse liver. Purified hPSC-derived hepatocytes also executed various liver metabolic functions in vitro, such as (1) storage of glucose in the form of glycogen and (2) conversion of free fatty acids into triglycerides. hPSC-derived hepatocytes stored ~4.5-to 5.6-fold greater glycogen than HepG2 and HuH7 cells (FIG. 4C). Triglyceride levels were ~6.9-fold higher in hPSC-derived hepatocytes relative to HepG2 cells (FIG. 4C). Additionally, hPSC-derived hepatocytes treated with high glucose and lipids accumulated lipid droplets (FIG. 4D). These results also suggest glycogen and triglycerides as possible energy sources for hPSC-derived hepatocytes, allowing them to withstand nutrient deprivation in the aforementioned metabolic selection strategy.

Additionally, hPSC-derived hepatocytes could engraft the injured liver of immunodeficient Fah/Rag2^{-/-}112rg^{-/-} (FRG) mice (FIG. 4E). FRG mice lack tyrosine metabolism enzyme Fah and thus develop chronic liver failure. 6 weeks after transplantation, hPSC-derived hepatocytes repopulated the mouse liver, as shown by the abundant number of human ALBUMIN+ hepatocytes in vivo (FIG. 4E). Of note, hPSC-derived hepatocytes were spatially distributed across the liver lobule, some of which were nearby blood vessels, such as the central and portal veins (FIG. 12E). hPSC-derived hepatocytes adjacent to the central vein expressed the pericentral hepatocyte marker glutamine synthetase (GS) (FIG. S4E). Hence, hPSC-derived hepatocytes could engraft the injured liver, attesting to their potential for liver regeneration.

hPSC-derived and primary hepatocytes can express interferon-stimulated genes in response to double-stranded RNA analog, unlike liver cancer cell lines. Furthermore, we explored the ability of hPSC-derived hepatocytes to activate interferon signaling, an innate immunity pathway with antiviral effects. Liver cancer cell lines HuH7 and HepG2 have been widely used to study Ebola and other viruses under BSL4 containment, yet they are defective in interferon production, which hampers their utility for studying viral infection in vitro.

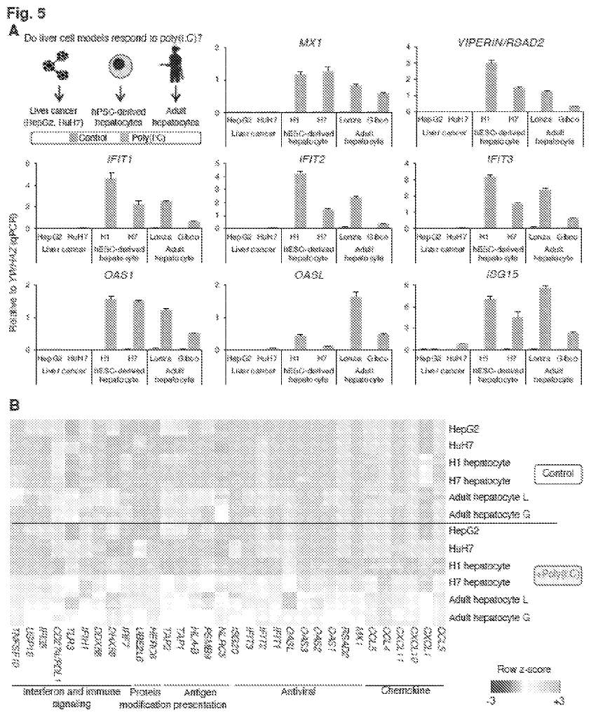

One day of treatment with poly(I:C), a double-stranded RNA analog, strongly induced interferon-stimulated genes (ISGs) MX1, VIPERIN, IFIT1, IFIT2, IFIT3, OAS1, OASL, and ISG15 in hPSC-derived hepatocytes obtained from two different hPSC lines, H1 and H7 (FIG. 5A). Similarly, poly(I:C) upregulated ISGs in primary adult human hepatocytes obtained from two different sources (FIG. 5A). As expected, poly(I:C) largely failed to upregulate these ISGs in HepG2 and HuH7 liver cancer cell lines (FIG. 5A). RNA-seq confirmed significant ISG and inflammatory cytokine upregulation in poly(I:C)-treated hPSC-derived and primary adult hepatocytes, but minimal changes in HepG2 and HuH7 cells (FIG. 5B). Taken together, hPSC-derived hepatocytes behaved more similarly to primary hepatocytes in their induction of ISGs than liver cancer cell lines. Having established that hPSC-derived hepatocytes can activate interferon signaling, we next applied these cells to model infection by Risk Group 4 viruses.

Risk Group 4 viruses can infect purified hPSC-derived hepatocytes. The liver is an important target of many deadly Risk Group 4 viruses. It has been known for over 30 years from autopsies of infected humans and macaques that liver cells are infected by Ebola virus, Sudan virus, Marburg virus, and Lassa virus in vivo.

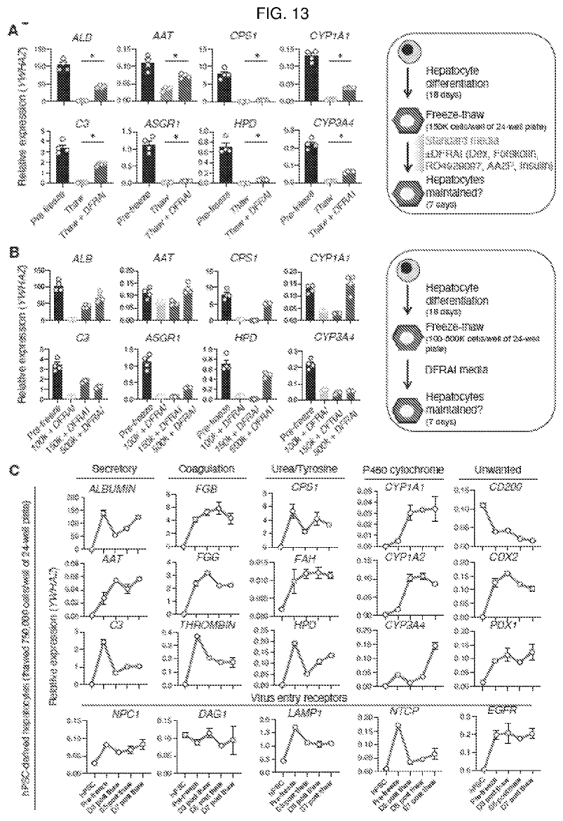

To test whether hPSC-derived hepatocytes could be infected by Risk Group 4 viruses, we first developed methods to cryopreserve, thaw, and culture hepatocytes before viral infection under BSL4 containment. However, thawed hPSC-derived hepatocytes cultured in standard commercially-available media rapidly lost the expression of hepatocyte genes (FIG. 13A). We hypothesized that hepatocytes could be maintained by continued exposure to hepatocyte-specifying signals, akin to those used to specify hepatocytes from hPSCs. Indeed, continued exposure to hepatocyte-specifying signals—glucocorticoid and PKA activation and NOTCH inhibition (FIG. 13A), together with high-density culture (FIG. 13B)—maintained hPSC-derived hepatocytes. Mechanistically, PKA and high cell density activate the Hippo pathway, which is required for hepatocyte maturation. In these conditions, thawed hPSC-derived hepatocytes could be cultured for 1 week while maintaining the expression of hallmark hepatocyte genes (FIG. 13C).

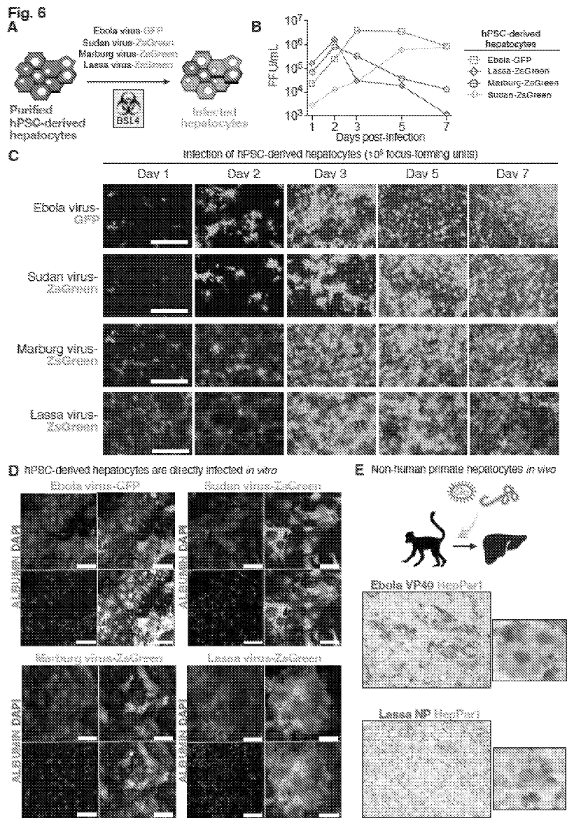

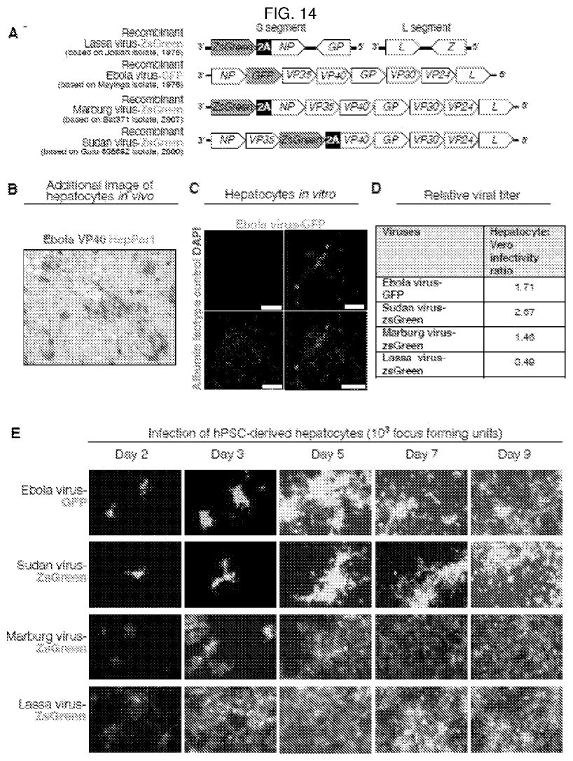

To visually track viral spread over time, we infected purified hPSC-derived hepatocytes with recombinant viruses wherein fluorescent proteins were inserted into authentic viral genomes: GFP-expressing Ebola virus, ZsGreen-expressing Sudan virus, ZsGreen-expressing Marburg virus, and ZsGreen-expressing Lassa virus (FIG. 6A, FIGS. 14A, 14E). Ebola, Sudan, Marburg, and Lassa virus extensively replicated in hPSC-derived hepatocytes, as shown by focus forming assays of the culture media (FIG. 6B) and direct fluorescent imaging of infected hepatocytes (FIG. 6C). We confirmed that these viruses infect hepatocytes, as cells co-expressed viral markers (ZsGreen or GFP) and the hepatocyte marker albumin (FIG. 6D, FIG. 14C). In sum, Ebola, Sudan, Marburg, and Lassa viruses can efficiently infect and replicate in hPSC-derived hepatocytes in vitro, providing a tractable platform to study the effects of these viruses on human hepatocytes.

Next, we molecularly confirmed that these Risk Group 4 viruses infect hepatocytes in vivo, using archived liver tissues from rhesus or cynomolgus macaques that had been previously infected by Ebola or Lassa virus. Immunohistochemistry revealed colocalization of hepatocyte marker HepPar1/CPS1 and viral antigens within hepatocytes in vivo (FIG. 6E, FIG. 14B), expanding on past morphologic observations that these viruses infect liver cells in vivo.

hPSC-derived hepatocytes responded to Ebola and Lassa viruses with divergent effects. What are the effects of Ebola and Lassa viruses on hepatocytes? Although arenaviruses and filoviruses both infect the liver and cause viral hemorrhagic fevers, there are significant differences in disease: Lassa virus (an arenavirus) has a ~1% fatality rate, whereas filoviruses (e.g., Ebola, Sudan, and Marburg viruses) cause extremely high fatality rates ranging from ~44-81%. To our knowledge, the effects of arenaviruses and filoviruses on human cells have yet to be compared side-by-side in the same experimental system. In subsequent experiments, we focused on comparing wild-type (i.e., non-recombinant) Ebola virus (a filovirus) and Lassa virus (an arenavirus). To this end, viral stocks were concentrated, purified, and titrated on hepatocytes to arrive at equivalent numbers of infected hepatocytes after Lassa or Ebola infection (FIG. 14D). Of note, hPSC-derived hepatocytes were differentially susceptible to these viruses compared to transformed monkey Vero cells ordinarily used to propagate and titrate these viruses, underscoring the importance of using physiologically-relevant cell-types for viral titration and multiplicity of infection (MOI) calculations (FIG. 14D).

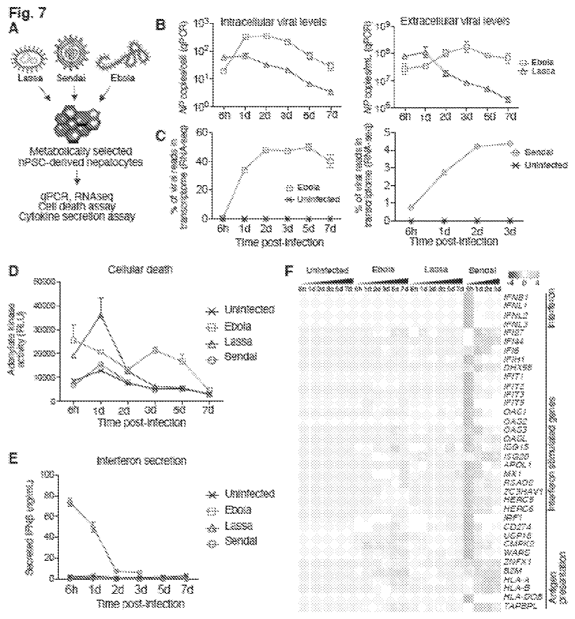

We infected purified hPSC-derived hepatocytes with high titers of purified authentic Ebola virus (Mayinga isolate) or Lassa virus (Josiah isolate) to synchronously infect >95% of hepatocytes (MOI~4) and analyzed transcriptional responses at 6 timepoints (6 hours and 1, 2, 3, 5, and 7 days post-infection). As a control, we also infected cells with Sendai virus (Cantell strain), a non-pathogenic paramyxovirus that stimulates interferon signaling. Intracellular and extracellular levels of Lassa virus peaked at 1 day post-infection (FIGS. 7A-B). Intracellular and extracellular Ebola virus levels peaked at 2 and 3 days post-infection, respectively (FIGS. 7A-B). Ebola virus replication was extensive: it occupied 48.2% of the hepatocyte transcriptome by 2 days post-infection as quantified by RNA-seq (FIG. 7C).

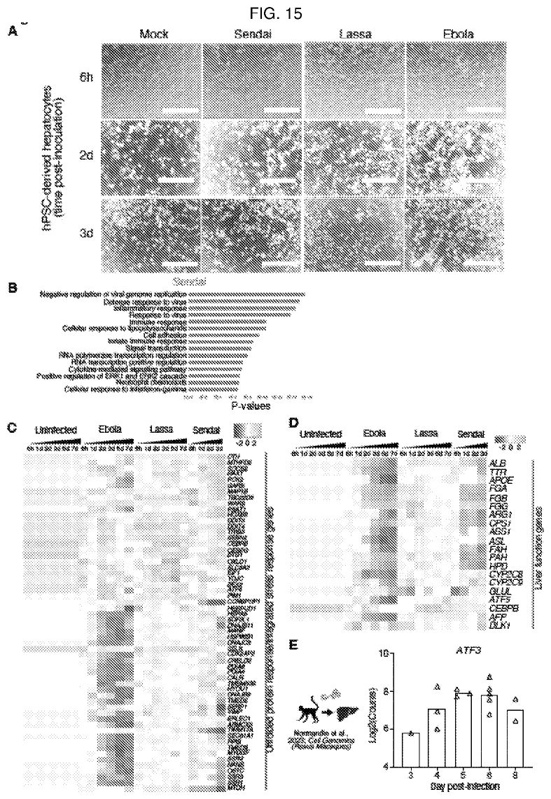

Ebola virus progressively killed hPSC-derived hepatocytes, as quantified by release of intracellular adenylate kinase into the culture media (FIG. 7D, FIG. 15A). By contrast, Lassa virus led to a transient wave of cell death at 1 day post—infection (FIG. 7D)—coincident with the highest levels of Lassa virus (FIG. 7B)—before subsequently re-normalizing.

Ebola and Lassa virus were remarkably effective at suppressing innate immunity in hepatocytes: despite high viral loads, there was almost undetectable secretion of IFNβ protein (FIG. 7E) or transcriptional induction of interferons, interferon target genes, or antigen presentation genes (FIG. 7F). This was notable, revealing the progressive destruction of Ebola virus-infected hepatocytes (FIG. 7D) was not accompanied by interferon production or responses. As a positive control, Sendai virus induced massive interferon secretion and signaling (FIGS. 7E-F, FIG. 15B), indicating that hPSC-derived hepatocytes are competent to produce interferon, but that Ebola and Lassa virus potently evade innate immune detection.

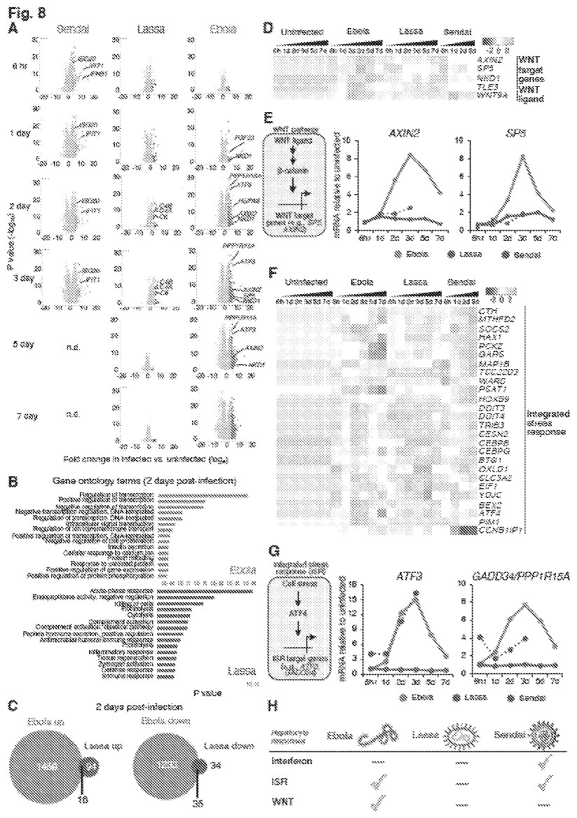

If Ebola and Lassa virus do not activate the interferon pathway, what are their transcriptional effects on hepatocytes? The effects of Lassa virus infection were transient and more limited (2 days post-infection, 110 genes upregulated >3 fold); they were associated with GO terms such as complement activation, proteolysis, cytolysis, and inflammation (FIGS. 8A-C), but subsequently largely renormalized within the next few days (by 5 days post-infection, 21 genes upregulated >3 fold). By contrast, Ebola virus infection significantly induced the expression of 1475 and 2202 genes after 2 or 5 days, respectively (FIGS. 8A, C). We found that Ebola virus and Lassa virus up-or downregulated largely different sets of genes (FIGS. 8B, C).

Remarkably, Ebola virus transcriptionally activated the WNT and integrated stress response (ISR) pathways in hepatocytes, whereas Lassa virus did not. First, Ebola virus induced the expression of WNT ligands (e.g., WNT9A) and WNT target genes (e.g., AXIN2, SP5, and NKD1) (FIGS. 8D-E). Liver injury rapidly activates WNT signaling to drive regeneration in vivo, suggesting that dying, Ebola virus-infected hPSC-derived hepatocytes may engage a pro-regenerative WNT program to compensate for cell death. Second, while Ebola virus did not detectably activate interferon signaling, we found that it instead strongly induced ISR signaling (FIGS. 8F-G, FIG. 15C). ISR is activated by cellular stresses, including viral infection and endoplasmic reticulum (ER) stress, and induces translational shutdown to blunt viral replication. Within 1 day, Ebola virus-infected hepatocytes upregulated a suite of ISR pathway target genes (e.g., DDIT3/CHOP, DDIT4, PIM1, EIF1, CCNB1IP1, GADD34/PPP1R15A), including ATF3, a key transcriptional activator of ISR signaling (FIGS. 8F-G, FIG. 15C).

Consistent with this in vitro finding, our analysis of published RNA-seq datasets of Ebola-infected non-human primates-which represent the gold standard model for Ebola virus disease-likewise revealed ATF3 and PPP1R15A were upregulated in the Ebola-infected primate liver (FIG. 8H, FIG. 15E). By contrast, ISR and WNT genes were not induced by Lassa virus in vitro, suggesting this is not a generic hepatocyte response to viral infection (FIGS. 8F-G, FIG. 15C). Sendai virus infection also induced certain ISR genes, some of which were different (GARS, HAX1, and WARS) than those induced by Ebola (CCNBP1IP1, YDJC, and PIM1), potentially reflecting activation of different ISR branches or differing levels of ISR activation (FIGS. 8F-G, FIG. 15C). Finally, Ebola virus and Sendai virus, but not Lassa virus, reduced expression of various liver function genes, including FGB, ALB, TTR, APOE, and ARG1 (FIG. 15D). Taken together, there are starkly different effects of Ebola virus and Lassa virus on purified human hepatocytes, especially with regard to cell death and activation of Wnt and ISR pathways (FIG. 8I).

Here we developed a new approach to create pure human hepatocytes in vitro from hPSCs, and we applied these purified hPSC-derived hepatocytes to discover how Ebola virus and Lassa virus affect hepatocytes. First, we differentiated hPSCs into ~80% pure hepatocytes, and pioneered a “metabolic selection” approach—withholding glucose, glutamine, and pyruvate—to subsequently deplete non-liver cells with high speed and specificity, providing a convenient method to purify hepatocytes from heterogeneous cell populations. Purified hPSC-derived hepatocytes were transcriptionally and functionally more akin to primary hepatocytes than liver cancer cell lines prevalently used in BSL4 virology. While Ebola and Lassa virus extensively infected hepatocytes in vivo and in vitro, we found that they led to starkly different effects. Ebola infection activated the WNT and ISR pathways in human hepatocytes, whereas Lassa did not. To our knowledge, this is the first time that different Risk Group 4 viral families (e.g., filoviruses vs. arenaviruses) have been directly compared in the same human experimental system, thus representing a step forward for comparative virology. In sum, purified hPSC-derived hepatocytes provide an abundant, experimentally accessible, and physiologically relevant model system for BSL4 virology. In turn, this will accelerate studies of the basic biology of these viruses and future therapeutic screens.

A roadmap for human liver differentiation. We comprehensively profiled stepwise changes in gene expression, chromatin accessibility, and cellular diversity of each step of hPSC differentiation into hepatocytes, using scRNAseq and OmniATACseq. This provides a rich resource to discover new markers and regulators of human liver differentiation. This resource also encompasses freshly-isolated and cultured adult hepatocytes and liver cancer cell lines (HepG2 and HuH-7), allowing for unbiased benchmarking of different liver cell culture models.

At each stage of differentiation, we provided lineage-specifying cues to generate the cell-type of interest while, of equal importance, blocking the signals that generated unwanted cell-types. scRNAseq revealed that this two-pronged approach rapidly and efficiently differentiated hPSCs into primitive streak, definitive endoderm, posterior foregut, and liver bud progenitors within 1, 2, 3, and 6 days of differentiation. Cell populations were mostly uniform at each of these steps, with little evidence of unwanted cell-types arising and reiterating the high precision of early differentiation (FIG. 9D).

By day 18 of differentiation, ~80% pure hepatocytes emerged, although we discovered they were commingled with intestinal goblet and neuroendocrine cells. We thus document the identity of non-liver cells that arise alongside hepatocytes during hPSC differentiation. Indeed, the liver and intestine are adjacent endodermal organs, and it is thus plausible for intestinal cells to erroneously appear during liver differentiation in vitro. The emergence of these non-liver cells necessitates approaches to exclusively purify hepatocytes, as heterogeneous cell populations pose challenges for various applications, including virology, drug screening, and regenerative medicine.

Metabolic selection: a new approach to purify hepatocytes. Purifying desired cell-types from heterogeneous cell populations remains a significant challenge for stem cell biology and regenerative medicine. To purify hepatocytes, we developed “metabolic selection,” a new approach to rapidly kill non-liver cells simply by withholding certain essential nutrients from the culture medium. This builds on the emerging concept that different cell-types require specific nutrients to survive. Indeed, hepatocytes are one of the few cell-types capable of converting glucose into glycogen, and can therefore rely on glycogen during glucose deprivation. Withholding glucose, pyruvate, and glutamine in HepSelect media for 1-3 days destroyed non-liver cells in vitro, while hepatocytes survived. Metabolic selection generated essentially pure hPSC-derived hepatocytes; scRNAseq could not overtly detect surviving intestinal or other cell-types. Importantly, metabolic selection purifies hepatocytes based on their metabolic functionality instead of surface marker expression. Metabolic selection thus provides a simple, scalable, and inexpensive method to purify hepatocytes.

Moreover, the combinatorial withdrawal of nutrients was critical to purify hepatocytes. Glucose removal was insufficient to eliminate intestinal cells (FIG. 11C). A previous study showed glucose removal was also insufficient to destroy hPSCs because they can utilize either glutamine or arginine to survive in glucose-depleted media. We found that simultaneously withholding glucose, pyruvate, and glutamine was critical to purify hepatocytes and eliminate non-liver cells. More broadly, metabolic selection can serve to purify other cell-types, including neurons, adipocytes, kidney, and skeletal muscle cells that also synthesize glucose de novo or rely on non-glucose sources to produce energy.

hPSC-derived hepatocytes provide an enhanced model system for BSL4 virology. The unique constraints of BSL4 experimentation have long posed a formidable challenge to understanding the mechanistic effects of Risk Group 4 viruses on human hepatocytes. Thus far, liver cancer cell lines such as HepG2 and HuH-7 have been extensively used in BSL4 virology, but cannot produce interferon and lack many hepatocyte features. hPSC-derived cell-types offer key advantages for BSL4 virology, as they can be produced in large numbers, yet are chromosomally normal, resemble their in vivo counterparts, and are not oncogenically transformed. However, one drawback of hPSC-derived cellular models is cellular heterogeneity, as hPSC differentiation often yields complex mixtures of multiple cell-types. Indeed, a pioneering study found that Ebola virus could infect differentiated hPSC populations that contained ~25% ALBUMIN+ hepatocytes but predominately contained non-liver cells. However, it was unclear whether hepatocytes and/or non-liver cells were infected, and whether any of the observed cellular responses could be attributed to non-liver cells within the population. Our ability to create purified human hepatocytes from hPSCs-which we showed can be extensively infected by Ebola virus, Sudan virus, Marburg virus, and Lassa virus-provides a more precise platform to study the detailed cellular effects of these Risk Group 4 viruses.

Effects of Ebola virus on human hepatocytes. Multiple Risk Group 4 viruses, including Ebola virus and Lassa virus, have a predilection to infect hepatocytes. While Ebola virus also infects other tissues in the body, liver infection has been implicated in hepatocyte death, coagulopathies, and liver injury that often accompanies end-stage disease. Hepatocytes secrete voluminous amounts of proteins (e.g., carrier and coagulation proteins) into the bloodstream, and much of their cell body is devoted to protein synthesis and secretion. We find that Ebola virus occupies ~50% of the hepatocyte transcriptome for up to a week, during which high levels of infectious Ebola virus particles are continuously released into the media. While immune cells are initial targets of Ebola virus in vivo, it is possible that once Ebola virus reaches the liver, it transforms hepatocytes into efficient virus production factories to achieve systemic viral dissemination and to overwhelm the host. Indeed, we and others previously discovered that the early extent of liver replication correlates with Ebola virus disease severity in humanized mice. In vivo, Ebola virus reaches extraordinary bloodborne levels within several days, which could partly reflect massive viral secretion by hepatocytes in the absence of effective immunological restraint of viral replication.

Ebola virus fails to trigger interferon and inflammatory cytokine expression by infected hepatocytes, despite massive viral loads (~50% of the transcriptome) for up to a week. High-level viral production in the virtual absence of interferon production may allow Ebola virus to stealthily replicate in, and be released from, infected hepatocytes without initially engaging immune defenses that might curb viral replication. Such interferon suppression is not a feature of all liver-tropic viruses: by contrast, hepatitis C virus potently induces interferon in hepatocytes. Additionally, Ebola virus-infected hepatocytes do not express inflammatory cytokines (e.g., CXCL1, CXCL10, and CXCL12) that recruit T cells, NK cells, and myeloid cells, which are instead strongly expressed by Sendai-virus infected hepatocytes. Initial impairment of immune cell recruitment into the liver may help explain the delayed immune responses observed in Ebola virus disease. Our findings that Ebola virus-infected hepatocytes minimally produce interferon or inflammatory cytokines may also help explain a longstanding curiosity in the field: there can be extensive hepatocyte necrosis, yet minimal inflammation, in Ebola virus-infected livers.

The conspicuous absence of interferon or inflammatory cytokine production after Ebola virus infection does not reflect a fundamental deficiency of our model system. We show that hPSC-derived hepatocytes are competent to secrete such cytokines in response to Sendai virus or poly(I:C) treatment, unlike liver cancer cell lines. However, our findings contrast with a recent study that infected a ~25% pure population of hPSC-derived hepatocytes with Ebola virus, and described interferon mRNA upregulation, although interferon protein levels were not assessed. The divergent results between our two studies could be attributed to unidentified, non-liver cells present within the culture, emphasizing the importance of generating pure hepatocytes. Overall, our results are generally consistent with autopsies that revealed surprisingly minimal inflammation in Ebola virus-infected livers.

While Ebola virus did not trigger interferon production, we found that hepatocytes nevertheless responded in two unexpected ways: activating the WNT and ISR pathways. First, we find that Ebola virus infection induces WNT signaling in hPSC-derived hepatocytes. Liver injury induces WNT signaling, thus triggering hepatocyte proliferation and regeneration. Our results could help explain human autopsy results that there is abundant hepatocyte proliferation in Ebola virus-infected livers, thought to reflect an ongoing regenerative response to cell death. Second, the ISR pathway can be triggered by multiple types of cellular stress, including double-stranded RNA (sensed by PKR) and ER stress, perhaps elicited by overproduction of viral proteins that cannot be correctly folded (sensed by PERK). The upstream drivers and downstream consequences of ISR pathway activation in Ebola virus infection are presently unknown. Classically, ISR activation leads to translational shutdown to curtail viral replication, which could present a cell-intrinsic mechanism for hepatocytes to reduce viral protein production despite failed interferon pathway activation. Additionally, excessive ISR signaling can eventually lead to cell death, providing a potential mechanism for Ebola virus-induced hepatocyte death that we observed.

Differences in how Ebola and Lassa viruses affect human hepatocytes. To our knowledge, this is the first study comparing the cellular effects of Risk Group 4 viruses from two different families—Ebola virus (a filovirus) vs. Lassa virus (an arenavirus)—side-by-side in the same human experimental system. Comparative virology, whereby the effects of different viruses are compared and contrasted, is a major pursuit of modern virology. The effects of different filoviruses have been compared. However, the effects of a Risk Group 4 filovirus (Ebola virus) vs. an arenavirus (Lassa virus) on human cells remain an open question.

Curiously, even after infection with a high viral dose (MOI≈4), Lassa virus induced modest and transient transcriptional changes in hepatocytes. Despite infecting up to 500,000 individuals every year in Africa, ~80% of Lassa cases are mild or asymptomatic. However, we find that despite high viral loads, Lassa is similar to Ebola virus in that it suppressed production of both interferon and inflammatory cytokines from infected hepatocytes. This may facilitate initial Lassa virus infection of hepatocytes, consistent with results from autopsies, while impairing early immune system engagement. However, we find that Lassa virus does not activate the WNT or ISR pathways, a key difference from Ebola. Our findings are altogether consistent with a hallmark autopsy study that although Lassa virus infects the human liver, its effects on the liver are generally more mild.

More broadly speaking, we demonstrate that purified hPSC-derived hepatocytes provide a powerful experimental system to generate and test mechanistic hypotheses regarding Risk Group 4 viruses, which would otherwise prove challenging in other model systems given the experimental constraints of BSL4 containment. hPSC-derived hepatocytes also constitute a platform to study and compare additional Risk Group 4 viruses, including nairoviruses (e.g., Crimean-Congo hemorrhagic fever virus) and New World arenaviruses (e.g., Junin virus) that infect human hepatocytes in vivo but whose cellular effects remain obscure.

### Materials and Methods

Cell culture. All cells in this study were cultured in standard incubator conditions (20% 02, 5% CO₂, and 37° C.).

Human pluripotent stem cell lines. The following human pluripotent stem cell (hPSC) lines were used in this study: wild-type H1, H7 and H9 hESCs (WiCell). H1 hPSCs are of a XY genotype, whereas H7 and H9 hPSCs are of a XX genotype.

Undifferentiated hPSCs were propagated in mTeSR Plus medium (StemCell Technologies)+1% penicillin/streptomycin (Thermo Fisher) or alternatively, mTeSR1 medium (StemCell Technologies)+1% penicillin/streptomycin in monolayer cultures, on Geltrex basement membrane matrix-coated plates (described below). For mTeSR Plus, the media was changed daily or every other day as per the manufacturer's recommendations. For mTeSR1, the media was changed daily. For brevity, we refer to mTeSR1 and mTeSR Plus interchangeably as “mTeSR” for the remainder of this Methods section.

To maintain cultures of undifferentiated hPSCs, when they became partially confluent, they were passaged by treating them for 7 minutes with EDTA (Versene, Thermo Fisher) at room temperature. Subsequently, EDTA was removed, mTeSR was added, and hPSCs were manually scraped off the plate to generate clumps. hPSC clumps were then seeded onto new plates precoated with Geltrex basement membrane matrix (described below) in mTeSR medium+1% penicillin/streptomycin. During EDTA-based maintenance passaging of undifferentiated hPSCs as clumps, ROCK inhibitor was not added. Alternatively, for differentiation experiments, hPSCs were instead dissociated using Accutase and seeded as single cells in ROCK inhibitor-containing media (described below).

To summarize, undifferentiated hPSCs were either passaged as clumps (using EDTA, without ROCK inhibitor) or as single cells (using Accutase, with ROCK inhibitor), respectively depending on whether cells were destined for continued undifferentiated maintenance or differentiation.

Liver cancer cell lines. HepG2 cells were gifted from Ee Chee Ren and HuH7 cells were purchased from Sekisui XenoTech. Both cell lines were cultured in DMEM+10% FBS+1% penicillin/streptomycin and, when confluent, were passaged 1:6 using TrypLE Express. HepG2 and HuH7 cells are transformed human cancer cell lines that harbor a XY genotype.

Vero E6 cells. Vero C1008 (Vero 76, clone E6, Vero E6) cells (European Collection of Authenticated Cell Cultures (ECACC), 85020206) were propagated in DMEM containing 2% FBS, 2 mM glutamine, and 1% penicillin/streptomycin and passaged at a 1:10 ratio through trypsinization. Vero E6 cells were used to propagate and titer viral stocks. Vero E6 cells represent a transformed African green monkey (Chlorocebus sabaeus) cell line of a XX genotype.

Mouse models. Immunodeficient Fah−/− Rag2−/−112rg−/−(FRG) mice on the NOD genetic background, obtained from Yecuris, were housed in the Lokey Stem Cell Research Building's Barrier Mouse Facility. FRG mice were fed with PicoLab High energy mouse diet (5LJ5) and given NTBC (Yecuris) at 8 mg per litre of drinking water. Adult FRG mice were transplanted with hPSC-derived hepatocytes (described below).

Non-human primate models. No living non-human primates were assessed as part of this study. In this work, we performed staining of archived livers from rhesus macaques (Macaca mulatta) that had been previously infected with Ebola virus and cynomolgus macaques (Macaca fascicularis) that had been previously infected with Lassa virus. These primates have been described in previous publications.

Ebola virus, Mayinga isolate. Ebola virus, Yambuku variant, Mayinga isolate (Ebola virus/Human/COD/1976/Yambuku-Mayinga; genomic sequence reported in NCBI accession number AF086833.2) was originally isolated by the CDC from a fatally-infected human in the Democratic Republic of the Congo in 1976, and was passaged on Vero E6 cells. Ebola virus is a member of the species Zaire ebolavirus; genus Ebolavirus; family Filoviridae; order Mononegavirales.

Lassa virus, Josiah isolate. Lassa virus, Josiah isolate (Lassa virus/Mouse/Sierra Leone/1976/Josiah; genomic sequences reported in NCBI accession numbers NC_004296.1 and NC_004297.1) was originally isolated by the CDC from the blood of an infected human in Sierra Leone in 1976, and was passaged on Vero cells. Lassa virus is a member of the species Lassa mammarenavirus; genus Mammarenavirus; family Arenaviridae; order Bunyavirales.

Ebola virus, Mayinga isolate, GFP-expressing. Recombinant GFP-expressing Ebola virus, Yambuku variant, Mayinga isolate (Ebola virus/Human-recombinant/COD/1976/Yambuku-Mayinga-GFP) was engineered by inserting a GFP cassette between the NP and VP35 genes of Ebola virus, Yambuku variant, Mayinga isolate (Ebola virus/Human/COD/1976/Yambuku-Mayinga; genomic sequence reported in NCBI accession number AF086833.2). Recombinant GFP-expressing Ebola virus was rescued from Vero cells transfected with respective viral plasmids, and was subsequently passaged on Vero E6 cells. Ebola virus is a member of the species Zaire ebolavirus; genus Ebolavirus; family Filoviridae; order Mononegavirales.

Sudan virus, Gulu-808892 isolate, zsGreen-expressing. Recombinant zsGreen-expressing Sudan virus, Gulu-808892 isolate (Sudan virus/Human-recombinant/UGA/2000/Gulu-808892-zsGreen) was engineered by inserting a zsGreen-P2A cassette immediately upstream of the VP40 gene of Sudan virus, Gulu-808892 isolate (Sudan virus/Human/UGA/2000/Gulu-808892; genomic sequence reported in NCBI accession number KR063670.1). Recombinant zsGreen-expressing Sudan virus was rescued from HuH7 cells transfected with respective viral plasmids, and was subsequently passaged on Vero E6 cells. Sudan virus is a member of the species Sudan ebolavirus; genus Ebolavirus; family Filoviridae; order Mononegavirales.

Marburg virus, Bat371 isolate, zsGreen-expressing. Recombinant zsGreen-expressing Marburg virus, Bat371 isolate (Marburg virus/Bat-recombinant/Uganda/2007/Bat371-zsGreen) was engineered by inserting a zsGreen-P2A cassette immediately upstream of the NP gene of Marburg virus, Bat371 isolate (Marburg virus/Bat/Uganda/2007/Bat371; genomic sequence reported in NCBI accession number FJ750958). In past work, isolate Bat371 has also been alternatively referred to as isolate 371Bat, sample ID 200704852, or viral isolate 811277. Recombinant zsGreen-expressing Marburg virus was rescued from BHK21 cells transfected with respective viral plasmids and was subsequently passaged on Vero E6 cells. Marburg virus is a member of the species Marburg marburgvirus; genus Marburgvirus; family Filoviridae; order Mononegavirales.

Lassa virus, Josiah isolate, zsGreen-expressing. Recombinant zsGreen-expressing Lassa virus, Josiah isolate (Lassa virus/Mouse-recombinant/Sierra Leone/1976/Josiah) was engineered by inserting a zsGreen-P2A cassette immediately upstream of the NP gene of the S genome segment of Lassa virus, Josiah isolate (Lassa virus/Mouse/Sierra Leone/1976/Josiah; genomic sequences reported in NCBI accession numbers HQ688673.1 and HQ688675.1). Recombinant zsGreen-expressing Lassa virus was rescued from BSR-T7/5 cells transfected with respective viral plasmids, and was subsequently passaged on Vero E6 cells. Lassa virus is a member of the species Lassa mammarenavirus; genus Mammarenavirus; family Arenaviridae; order Bunyavirales.

Sendai virus, Cantell strain. The original provenance of Sendai virus, Cantell strain is unknown, but it was passaged over 100 times in chicken embryonated eggs at the Central Public Health Laboratory in Helsinki, Finland. Sendai virus is a member of the species Murine respirovirus; genus Respirovirus; family Paramyxoviridae; order Mononegavirales. Sendai virus, Cantell strain was obtained from ATCC (VR-907).

### Method Details

Human pluripotent stem cell culture. As described above, undifferentiated hPSCs were maintained in either mTeSR1 or mTeSR Plus media supplemented with 1% penicillin/streptomycin. When partially confluent, hPSCs were passaged as clumps by dissociating them for 7 minutes using EDTA (Versene), followed by manual scraping, and then plating onto Geltrex-coated wells for continued maintenance of undifferentiated hPSCs.

Coating cell culture plastics with Geltrex basement membrane matrix. Geltrex (Thermo Fisher) was thawed at 4° C. and diluted 1:100 in cold DMEM/F12 medium, generating Geltrex working stocks, which were stored long-term at −20° C. As needed, Geltrex working stocks were thawed at 4° C., and used to coat cell culture plastics by adding half the working volume typically used for that cell culture plate. For instance, 1 mL of Geltrex working stock was added per well of a 6-well plate, or 0.5 mL Geltrex was added per well of a 12-well plate. Geltrex coating was performed for a minimum of 1 hour at 37° C., during which it polymerized to form a thin film on the bottom of the plate. The excess Geltrex was then aspirated immediately before plating cells on the Geltrex-coated plate.

Primary human hepatocyte culture. Primary human hepatocytes (obtained from XenoTech, Lonza, or Gibco) were seeded onto rat tail collagen 1-coated plates and cultured in human hepatocyte cell culture media (Lonza).

Preparation of CDM2, CDM3, CDM4B, HepSelect, and LipidHep basal medium for hPSC differentiation. The composition of CDM2 basal medium has been described previously: 50% v/v IMDM+ GlutaMAX (Thermo Fisher, 31980-097)+50% v/v F12+ GlutaMAX (Thermo Fisher, 31765-092)+1 mg/mL polyvinyl alcohol (Sigma, P8136-250G)+1% v/v chemically defined lipid concentrate (Thermo Fisher, 11905-031)+450 μM 1-thioglycerol (Sigma, M6145-100 ML)+0.7 μg/mL recombinant human insulin (Sigma, 11376497001)+15 μg/mL human transferrin (Sigma, 10652202001)+1% v/v penicillin/streptomycin (Thermo Fisher, 15070-063). Polyvinyl alcohol was brought into suspension by gentle warming and magnetic stirring.

The composition of CDM3 basal medium has been described previously: 45% v/V IMDM+ GlutaMAX (Thermo Fisher, 31980-097)+45% v/v F12+ GlutaMAX (Thermo Fisher, 31765-092)+10% v/v KnockOut Serum Replacement (Thermo Fisher, 10828028)+1 mg/ml polyvinyl alcohol (Sigma, P8136-250G)+1% v/v chemically defined lipid concentrate (Thermo Fisher, 11905-031)+1% v/v penicillin/streptomycin (Thermo Fisher, 15070-063). Polyvinyl alcohol was brought into suspension by gentle warming and magnetic stirring.

The composition of CDM4B basal medium is as follows: 100% low glucose DMEM+ pyruvate+L-glutamine (Thermo Fisher, 11885084)+15 μg/mL human transferrin (Sigma, 10652202001)+1% v/v penicillin/streptomycin (Thermo Fisher, 15070-063). Exogenous insulin was not included as part of the basal medium, but was instead supplemented as one of the differentiation factors, as described below. The main differences between CDM4B (this study) and the previously-described CDM4 basal medium are (1) the use of low glucose DMEM (instead of F12 and IMDM, which contain high glucose), (2) withholding concentrated amino acid supplement, and (3) withholding concentrated lipids.

The composition of HepSelect basal medium is as follows: 100% DMEM lacking glucose, glutamine, phenol red, and pyruvate (Thermo Fisher, A1443001)+15 μg/mL human transferrin (Sigma, 10652202001)+1% v/v penicillin/streptomycin (Thermo Fisher, 15070-063). HepSelect media, lacking multiple essential nutrients, was used for metabolic selection to purify hepatocytes by killing non-liver cells.

The composition of LipidHep basal medium is as follows: 50% v/v IMDM+ GlutaMAX (Thermo Fisher, 31980-097)+50% v/v F12+ GlutaMAX (Thermo Fisher, 31765-092)+1% v/v chemically defined lipid concentrate (Thermo Fisher, 11905-031)+15 μg/mL human transferrin (Sigma, 10652202001)+1% v/v penicillin/streptomycin (Thermo Fisher, 15070-063). LipidHep media, which is glucose- and lipid-rich, was used to induce lipid droplet formation in hepatocytes.

All media was sterilely filtered (through a 0.22 μm filter) prior to use. The compositions of these respective basal media are summarized as follows:

| | | | | | HepSelect | |
|---|---|---|---|---|---|---|
| | | | | CDM4B | 100% DMEM | |
| | CDM2 | | CDM3 | 100% low | lacking | LipidHep |
| | 50% IMDM + | | 45% IMDM + | glucose | glucose, | 50% IMDM + |
| | GlutaMAX, | | GlutaMAX, | DMEM + | glutamine, | GlutaMAX, |
| | 50% F12 + | | 45% F12 + | pyruvate + L- | phenol red, | 50% F12 + |
| | GlutaMAX | | GlutaMAX | glutamine | and pyruvate | GlutaMAX |
| Polyvinyl | 1 | mg/mL | 1 mg/mL | | | |
| alcohol | | | | | | |
| Chemically | 1% | | 1% | | | 1% |
| defined lipid | | | | | | |
| concentrate | | | | | | |
| 1-thioglycerol | 450 | μM | | | | |
| Insulin | 0.7 | μg/mL | | | | |
| Transferrin | 15 | μg/mL | | 15 μg/mL | 15 μg/mL | 15 μg/mL |
| Penicillin/ | 1% | | 1% | 1% | 1% | 1% |
| streptomycin | | | | | | |
| KnockOut | | | 10% | | | |
| Serum | | | | | | |
| Replacement | | | | | | |

hPSC differentiation into hepatocytes: Steps 0-7. hPSCs were differentiated into hepatocytes generally following a previous protocol but with modifications as described below. Differentiation media was changed every 24 hours.

Seeding hPSCs for differentiation (Step 0). In contrast to passaging hPSCs for maintenance, a different passaging procedure was used to plate hPSCs for differentiation. Notably, seeding of hPSCs as single cells is paramount for efficient differentiation. To seed hPSCs for differentiation, undifferentiated hPSCs were dissociated into single cells (Accutase, Thermo Fisher) and plated into recipient wells in mTeSR supplemented with Thiazovivin (1 μM, Tocris; a ROCK inhibitor, to enhance hPSC survival after passaging) onto plates precoated with Geltrex basement membrane matrix, thus plating ~45,000-79,000 hPSCs/cm² (i.e., ~170,000-300,000 hPSCs/well of a 12-well plate; typically ~250,000 hPSCs/well of a 12-well plate). Freshly-seeded hPSCs were allowed to adhere and recover for 24 hours in mTeSR+1 μM Thiazovivin before initiating differentiation, during which the hPSCs re-formed small clumps. To reiterate, hPSCs are maintained by passaging as clumps (to maintain normal karyotype) but are seeded for differentiation as single cells (to enable efficient differentiation).

Day 1 (anteriormost primitive streak induction, 24 hours [Step 1]). Day 0 hPSCs were briefly washed (DMEM/F12, Thermo Fisher) to remove all traces of mTeSR+ Thiazovivin. Then, they were differentiated towards anteriormost primitive streak in CDM2 media supplemented with Activin A (100 ng/mL, R&D Systems), CHIR99021 (3 μM, Tocris), FGF2 (20 ng/ml, Thermo Fisher), and PI-103 (50 nM, Tocris) for 24 hours, as previously described, thereby yielding day 1 anteriormost primitive streak.

Day 2 (definitive endoderm induction, 24 hours [Step 2]). Day 1 anteriormost primitive streak cells were briefly washed (DMEM/F12) and then differentiated towards definitive endoderm in CDM2 media supplemented with Activin A (100 ng/mL), LDN-193189 (250 nM, Tocris), and PI-103 (50 nM) for 24 hours, as previously described, thereby yielding day 2 definitive endoderm.

Day 3 (foregut induction, 24 hours [Step 3]). Day 2 definitive endoderm cells were briefly washed (DMEM/F12) and then differentiated towards foregut endoderm in CDM3 media supplemented with BMP4 (30 ng/ml, R&D Systems), TTNPB (75 nM, Tocris), A-83-01 (1 μM, Tocris), and FGF2 (20 ng/ml) for 24 hours, as previously described, thereby yielding day 3 foregut endoderm.

Days 4-5 (early liver induction, 48 hours [Step 4]). Day 3 foregut cells were briefly washed (DMEM/F12) and then differentiated in CDM3 media supplemented with Forskolin (1 μM, Tocris), C59 (1 μM, Tocris), BMP4 (30 ng/ml), and Activin (10 ng/ml) for 48 hours, as previously described, thereby yielding day 5 early liver cells.

Day 6 (liver bud progenitor induction, 24 hours [Step 5]). Day 5 early liver progenitor cells were briefly washed (DMEM/F12) and then differentiated in CDM3 media supplemented with Forskolin (1 μM), CHIR99201 (1 μM), BMP4 (30 ng/ml) and Activin (10 ng/ml) for 24 hours, as previously described, thereby yielding day 6 liver bud progenitors.

Days 7-12 (early hepatocyte induction, 6 days [Step 6]). Day 6 liver bud progenitor cells were briefly washed (DMEM/F12) and then differentiated in CDM4B media supplemented with Forskolin (10 μM), Dexamethasone (10 μM, Tocris), RO4929097 (2 μM, Cellagen), AA2P (200 μg/mL, Cayman), Insulin (10 μg/mL, Sigma), BMP4 (10 ng/ml), and OSM (10 ng/ml, R&D Systems) for 6 days, thereby yielding day 12 early hepatocytes. This was performed essentially as previously described, with the exception that CDM4B basal medium was used in lieu of CDM4.

Days 13-18 (hepatocyte induction, 6 days [Step 7]): Day 12 early hepatocytes were briefly washed (DMEM/F12) and then differentiated in CDM4B media supplemented with Forskolin (10 μM), Dexamethasone (10 μM), RO4929097 (2 μM), AA2P (200 μg/mL), and Insulin (10 μg/mL) for 6 days, thereby yielding day 18 hepatocytes. This was performed essentially as previously described, with the exception that CDM4B basal medium was used in lieu of CDM4.

Metabolic selection of day 18 hPSC-derived hepatocytes. Day 18 hPSC-derived hepatocytes were briefly washed with DMEM lacking glucose, glutamine, and pyruvate (Gibco, 11966025), and then cultured in HepSelect basal medium supplemented with Forskolin (10 μM), Dexamethasone (10 μM), RO4929097 (2 μM), AA2P (200 μg/mL), and Insulin (10 μg/mL) for 1-3 days. As described above, HepSelect medium lacks glucose, glutamine, and pyruvate, and therefore this medium purifies hepatocytes by killing non-liver cells.

Prior to freezing, metabolically-selected hepatocytes were first cultured in recovery media replete with glucose, glutamine, and pyruvate: CDM4B basal medium supplemented with Forskolin (10 μM), Dexamethasone (10 μM), RO4929097 (2 μM), AA2P (200 μg/mL), and Insulin (10 μg/mL), for 1 day. This nutrient-replete recovery media was identical to the hepatocyte induction media used on days 13-18 of hPSC differentiation. After this 1 day of recovery in nutrient-replete media, hepatocytes were then dissociated and frozen.

Cryopreservation of hPSC-derived hepatocytes. hPSC-derived hepatocytes were dissociated using 0.5-1 mL of either Accutase or TrypLE Express per well of a 12-well plate for 5-10 minutes or until cells were detached from the cell culture plate. The remaining cells were carefully mixed 1-2 times using a P1000 pipette in order to generate single-cell suspension, with care to avoid excessive pipetting, which adversely affected cell survival. The cell suspension from each well of a 12-well plate was diluted in 14 mL of DMEM/F12 media, and the number of cells was counted. The cell suspension was centrifuged for 5 minutes at 4° C. at 300 g, and the supernatant was removed. The cell pellet was resuspended in freezing media (10% DMSO+90% FBS; sterilely filtered prior to use) at a concentration of 1-2 million cells/1 mL of freezing media. hPSC-derived hepatocytes were transferred in a cryopreservation container to the −80° C. freezer for a minimum of 24 hours before being transferred to a liquid N₂ tank for long-term storage.

Thawing of hPSC-derived hepatocytes. Frozen vials of hPSC-derived hepatocytes were removed from the liquid N₂ tank, briefly placed on a bed of dry ice, and then partially submerged in a 37° C. water bath until most of the ice had melted. The contents of each vial, consisting of 1 mL of thawed hepatocytes in freezing medium, was then transferred to a 15 ml conical tube containing 14 mL of DMEM/F12 media at room temperature. After centrifugation for 5 minutes at 300 g, the supernatant was aspirated, and the cell pellet was gently resuspended in hPSC-derived hepatocyte maintenance medium, which comprised Lonza Hepatocyte Culture Media+10 μM Dexamethasone+10 μM Forskolin+2 μM RO4929097+200 μg/ml AA2P+10 μg/ml Insulin+2 μM Thiazovivin (DFRAIT). Thawed hepatocytes were cultured on Geltrex-coated 24-well plates. For adequate maintenance of hepatocyte identity post-thaw, hepatocytes were seeded at a high density of ~500,000-750,000 cells per well of a 24-well plate. Both DFRAIT and high cell density were crucial to preserve the identity of hPSC-derived hepatocytes after thawing. Under such conditions, thawed hPSC-derived hepatocytes could be maintained for 6 days while largely continuing to express cardinal hepatocyte marker genes.

Alternatively, in some experiments, hPSC-derived hepatocytes were thawed in Lonza Hepatocyte Culture Media+ DFRAIT that was further supplemented with Chroman 1 (50 nM, MedChem Express), Emicrasan (5 μM, Tocris), Polyamine (1:1000, Sigma-Aldrich), and Trans-ISRIB (0.7 μM, Tocris). This “CEPT” cocktail has been previously reported to enhance cell survival in stressful environments.

Immunostaining of hPSC-derived hepatocytes. Media was aspirated from cultured cells and the cells were rinsed with DMEM. Then, cells were fixed with 4% paraformaldehyde in 1×PBS for 20 minutes at room temperature. After fixation, the cells were washed 3 times with 1×PBS. Then, the cells were blocked using a blocking buffer (10% donkey serum+0.1% Triton X in 1×PBS) for one hour at room temperature. Next, the cells were stained with primary antibodies (see reagents section) in 1% donkey serum containing 0.1% Triton X in 1×PBS overnight at 4° C. The next day, the cells were washed 3 times with 0.1% Triton x in 1×PBS for 5 minutes each. After, the cells were stained with fluorophore-conjugated secondary antibodies (see reagents section) diluted in 1:1000 (1% donkey serum+0.1% Triton X in 1× PBS) in the dark for one hour at room temperature. Finally, the cells were washed once with 100 ng/ml DAPI in PBS, twice with 0.1% Triton X PBS for 5 minutes and then stored in 1× PBS. Images were acquired using Zeiss LSM 800 confocal microscope and analyzed using Fiji (Image J).

Mouse tissue section immunostaining. Liver tissues from FRG mice transplanted with hPSC-derived hepatocytes were isolated, and then fixed in 4% paraformaldehyde in 1×PBS. After fixation, slides were washed twice with 0.1% Tween 20 in PBS for 15 minutes each. Next, slides were incubated in 0.25% Triton X in PBS for 10 minutes. Then, the slides were blocked using a blocking solution (5% donkey serum in 0.25% Triton X) for 1 hour at room temperature. After blocking, slides were stained with primary antibodies (see reagents section) in blocking solution overnight at 4° C. The next day, slides were washed with 0.25% Triton X three times for 15 minutes each. Then, secondary antibodies (see reagents section) were added at 1:1000 into the blocking solution for 1 hour. After secondary antibody incubation, slides were washed once with 100 ng/mL of DAPI in 0.25% Triton X in PBS for 15 minutes. Then, the slides were washed twice with 0.25% triton X in PBS for 15 minutes each. After washing, slides were mounted onto glass coverslips using Prolong Gold Antifade Mountant (Thermo Fisher Scientific P10144). Slides were air-dried at room temperature in the dark overnight. Images were acquired using Zeiss LSM 800 confocal microscope.

Flow cytometry analysis of surface marker expression on live cells. Cells were dissociated using TrypLE Express, pelleted, resuspended in FACS buffer (0.5% BSA Fraction V+5 mM EDTA in PBS), and strained through a 100 μm filter (BD Biosciences) to generate a single-cell suspension. The cell suspension was aliquoted into individual tubes and stained with anti-CD200-APC or anti-ASGR1-PE antibodies for 30 minutes on ice in the dark. Subsequently, the cells were washed 3 times with 1-2 mL of FACS buffer, resuspended in 300 μL of FACS buffer containing 100 ng/ml DAPI into a FACS tube and analyzed by FACS using BD LSR Fortessa X20. FCS files were analyzed using Flowjo.

To sort Clover+ and Clover− cells generated from FAH-2A-Clover hPSCs by fluorescence-activated cell sorting (FACS), cells were dissociated as single cells and stained with DAPI before FACS. Separate samples of FAH-Clover+ and Clover− populations were gated and collected and then harvested for gene expression analyses.

High throughput antibody screen of FAH-2A-Clover hPSC reporter line. The LegendScreen kit (Biolegend, 700007) containing 361 PE-conjugated monoclonal antibodies was used to identify diagnostic cell surface markers of hPSC, day 6 hPSC-derived liver progenitors and day 18 FAH-2A-Clover hPSC-derived hepatocytes. These cells were dissociated using TrypLE Express to generate single-cell suspension. Lyophilized antibodies are reconstituted in 25 μL deionized water before their use to stain cells following the manufacturer's instructions. The cell suspension was filtered through a 100 μm strainer to remove clumps. 75 μL cell suspension was added to each antibody-containing well, pipette-mixed 3 times and incubated in the dark at 4° C. for 20-30 mins. The cells were pelleted at 500 g for 6 minutes and the supernatant was discarded by plate inversion. The cells were washed twice with 200 μL cell staining buffer by pipette mixing, resuspended in DAPI-containing cell staining buffer and analyzed with a BD LSR Fortessa X20 flow cytometer.

Single-cell RNA-sequencing library preparation. Single cells were dissociated using TrypLE Express and resuspended in cold PBS containing 0.04% BSA. Cells were strained in 40-70 μm strainers, mixed 1:1 with trypan blue and counted using a hemocytometer to determine the number of live cells. Libraries were prepared from the dissociated cells using the Chromium Single Cell 3′ Reagent Kit (10× Genomics), following the manufacturer's instructions. All samples were multiplexed using 10× Genomics' Single Index Kit T Set A, quality control (QC) was performed using the Agilent Bioanalyzer high sensitivity chip, libraries were pooled in equal proportions and then sequenced on the NovaSeq 6000 S4 (Illumina).

Single-cell RNA-sequencing computational analysis and quality control. After conducting deep sequencing of 10× Genomics single-cell RNA-sequencing libraries, raw FASTQ reads were aligned to human reference genome GRCh38 and filtered using CellRanger 6.1.2. Molecular (unique molecular identifier [UMI]) and cellular barcodes were counted. The resultant cell matrices were imported and analyzed using Seurat 4.0.4 (Hao et al., 2021). We performed quality control: poor-quality cells that were dead/dying, had low gene counts, or had high mitochondrial counts were computationally excluded. Putative doublets with significantly higher numbers of expressed genes were also removed. Quality control was performed on each individual library before data from each library was merged into a Seurat object.

The data were then normalized using SCTransform (Hafemeister and Satija, 2019), mitochondrial content regressed using vars.to.regress, and overdispersion was estimated and modeled using glmGamPoi (Ahlmann-Eltze and Huber, 2020). Principal component analysis (PCA), neighbor finding, and clustering were performed before visualization in a Uniform Manifold Approximation and Projection (UMAP) plot (Becht et al., 2018). Heatmaps detailing the expression of selected markers, as well as the top 10 enriched genes marking each cluster, were plotted. Markers enriched in individual clusters were identified using FindAllMarkers, with parameters min.pct=0.25.

Comparing in vitro vs. in vivo hepatocytes using single-cell RNA-sequencing. To compare the transcriptional similarity between metabolically selected hPSC-derived hepatocytes and adult hepatocytes, the average expression of each gene in the SCTransformed object was calculated using the “AverageExpression” function in Seurat 4.0.4. All genes in this merged dataset were used in the “CellScatter” function to calculate Pearson correlation coefficients between fresh human adult hepatocytes, cultured human adult hepatocytes, HepG2 liver cancer cells, HuH7 liver cancer cells, and hPSC-derived hepatocytes.

For the calculation of module scores, the “Addmodulescore” function in Seurat 4.0.4 was used. Liver-specific markers defining hepatocytes were chosen to score “metabolically-selected” hPSC-derived hepatocytes, adult hepatocytes, cultured adult hepatocytes, HepG2, and HuH7 cells. The liver markers used were obtained from the Tabula Muris Consortium, which performed single-cell RNA-sequencing of hundreds of different adult cell-types and defined the following hepatocyte-enriched markers: Alb, Apoa1, Ass1, Cdh1, Cyp2e1, Glul, Gstp1, Gulo, Hsl, Hamp, Oat, Pck1, Serpina1c, Ttr, Ubb (Tabula Muris et al., 2018).

Cell-type identification from single-cell RNA-sequencing data. At day 18 of hPSC differentiation, six clusters of cells were identified by scRNAseq by Louvain clustering. Of these, three clusters were predominately negative for ALBUMIN and were thus classified as non-hepatocytes, which were putatively annotated based on expression of the following markers: Intestinal goblet-like cells: This cluster expressed known goblet cell markers, including CREB3L1 (Asada et al., 2012), TFF1 and TFF3 (Aihara et al., 2015; Wang et al., 2019), REG4 (Wang et al., 2019) and AGR2 (Park et al., 2009). Additionally, some of the top distinguishing genes of this cluster—including RNASE1, RAMP1, QSOX1, ASPH, CREB3L1, REG4, AGR2, GOLM1, S100A6, GALNT12, FKBP11 and LIPH—are relatively specific goblet cell markers, as revealed by our perusal of mouse adult intestinal scRNAseq data.

Intestinal enteroendocrine-like cells: This cluster expressed known enteroendocrine cell markers, including NEUROD1, NKX2.2, INSM1 and CHGA (Beumer et al., 2020; Egozi et al., 2021; Engelstoft et al., 2013; Haber et al., 2017). Concerning markers known to identify discrete subsets of enteroendocrine cells, this cluster predominately expressed markers of X (GHRL, MLN, ARX, ISL1) and K/L (TMEM190, SCG5) subtypes, but not enterochromaffin (TPH1) or N/I subtypes (SST, CCK, NTS, PYY) (Egozi et al., 2021). Many of these enteroendocrine markers—including NEUROD1, INSM1, CHGA, GHRL, ARX, ISL1 and SCG5—are specific enteroendocrine cell markers, as revealed by our perusal of mouse adult intestinal scRNAseq data.

Mesenchymal-like cells: This cluster, which was present at very low frequency, expressed LUM, COL3A1, COL1A1, TAGLN, HAND1, and ISL1.

RNA extraction and bulk-population RNA-seq. Cells were lysed using Zymo RNA lysis buffer and total RNA was extracted from cells using the Zymo Quick RNA kit per the manufacturer's instructions. High-quality RNA with RNA integrity number (RIN)>7 was used for library construction. For each cell-type, we included 2-3 technical replicates, generally defined as different wells of the same cell-type generated in the same differentiation experiment. In one experiment, 46 RNA-seq libraries were multiplexed and then sequenced on 2 lanes of the Illumina NovaSeq 6000 S4 sequencer by Novogene to generate 150-bp paired-end reads. In a second experiment, 36 RNA-seq libraries were multiplexed and then sequenced on 1 lane of the NovaS4 sequencer by Novogene to generate 150-bp paired-end reads.

The quality of RNA-seq reads were assessed using FastQC. Next, using TrimGalore and a stringency setting of 3, paired-end reads were trimmed to remove index adaptors and reads shorter than 20 bp. Low quality base calls (Phred score <33) were also eliminated. Using Kallisto, the trimmed reads were pseudoaligned with the human GRCh38 reference genome and gene-level RNA-seq counts were quantified (Bray et al., 2016). The counts were analyzed using DESeq2 which estimates the size factors, dispersions, and fits the count data into a negative binomial distribution model (Love et al., 2014). The counts were normalized using regularized log transformation to remove the dependence of variance on the mean of all samples.

Differentially expressed genes (DEGs) were identified using threshold of adjusted p-value <0.01 and logzfold changes >1. DEGs enriched in day 18 hPSC-derived hepatocytes compared with H1 hPSCs were used as inputs for functional annotation using the Database for Annotation, Visualization, and Integrated Discovery (DAVID) v6.8. This yielded enriched Gene Ontology terms in biological processes, of which selected metabolic pathways were plotted in a bar plot. A volcano plot showing genes differentially expressed between non-selected (day 19) vs. metabolically-selected hPSC-derived hepatocytes (day 19) was shown; genes showing log₂ fold expression differences less than 3 were represented in gray while differentially expressed genes were highlighted in distinct colors.

Analysis of published RNA-seq datasets. RNA-seq data was obtained from Normandin et al., 2023 and the counts were log₂ normalized using DESeq2 R package. BioMart R package was used to annotate gene names using the emsembl M. mulatta database. Ensembl IDs without annotated gene names were removed from this analysis.

Raw files were downloaded from Jankeel et al., 2020 using Prefetch and Fasterq-dump, and end reads were trimmed using TrimGalore. The reads were then aligned to the M. Fascicularis genome (release 108) using STAR aligner. The unstranded gene counts were then analysed using DESEQ2 (see RNA extraction and bulk population RNA-seq).

OmniATAC-seq library construction. OmniATAC-seq of hPSC-derived cell-types was performed largely as previously described (Corces et al., 2017). First, buffers including resuspension buffer (10 mM Tris-HCl, pH 7.5, 10 mM NaCl, 3 mM MgCl₂, nuclease-free H₂O) cold lysis buffer (resuspension buffer+0.1% v/v NP-40+0.1% v/v Tween-20+0.01% v/V Digitonin) and wash buffer (99.9% resuspension Buffer+0.1% v/v Tween-20) was prepared.

hPSC-derived cell-types were dissociated into single cells with TrypLE Express, and counted using a hemocytometer. Each sample has two replicates. For cell lysis and nuclei pelleting, 50,000 cells from each hPSC-derived cell-type were pelleted and washed with 500 μL cold PBS before lysis in 100 μL cold lysis buffer for 3 mins on ice. To the cell lysate, 1 mL of cold wash buffer was added. The mixture was centrifuged at 500 g for 10 minutes at 4° C. Then the supernatant (cytoplasm) was discarded and the pellet (nuclei) was kept.

Transposition. Transposition reaction mix from the Nextera DNA library prep kit (1× Tagment DNA Buffer, 1× PBS, 0.1% v/v Tween-20, 0.01% v/v Digitonin, and Tn5 Transposase [Tagment DNA Enzyme 1] in nuclease-free H₂O) was added to the pellet to resuspend nuclei. The cell transposition mix was incubated at 37° C. for 30 minutes on a thermal mixer, shaking at 1,000 rpm.

DNA purification and amplification. Next, DNA was purified using Qiagen MinElute Reaction Cleanup Kit. DNA was then PCR amplified using 15 and 17 index primers on a thermal cycler.

Library purification. Libraries were then purified using AMPure XP beads, multiplexed, underwent quality control assessments, and then sequenced using the NovaSeq 6000 S4 (Illumina) at a depth of ~2.5 billion reads per lane. In our study, we generally sequenced OmniATACseq libraries at a depth of >200 million raw reads per library, in keeping with guidelines that >50 million reads are needed to identify accessible chromatin elements and >200 million reads are needed to identify enriched transcription factor motifs.

OmniATACseq computational analysis and identification of hepatocyte-accessible chromatin elements. Sequenced OmniATACseq libraries were computationally processed using the ENCODE ATAC-seq analysis pipeline. Reads were aligned to human genome hg38 using Bowtie2 (Langmead and Salzberg, 2012). Peaks were called using MACS2 for each library (Zhang et al., 2008). A unified peak list for each cell-type was generated by selecting only peaks that were reproducible between both replicates. This was achieved through an irreproducible discovery rate (IDR) analysis at the threshold of 0.05 described by the ENCODE Consortium (Gerstein et al., 2012). Finally, peaks that overlapped with a “blacklist” of artifactual regions in the hg38 genome were filtered.

Diffbind (Ross-Innes et al., 2012) was used to identify chromatin regions that exhibited >8-fold (i.e., >2³-fold) differential accessibility between hPSC and differentiated hPSC-derived hepatocytes. Analyzing these hepatocyte-accessible and hPSC-accessible chromatin elements, including repeat-masked sequences and 200 bp regions, we discovered overrepresented DNA motifs using HOMER findMotifsGenome.pl (Heinz et al., 2010). Using a similar approach (as described above), chromatin elements differentially accessible between hPSC and other cell types (e.g. Day 1 hPSC-derived primitive streak, day 2 hPSC-derived definitive endoderm, day 3 hPSC-derived posterior foregut, day 6 hPSC-derived liver bud progenitors, day 12 hPSC-derived hepatic progenitors) were determined. In sum, enriched motifs were determined for 18 sample groups, with 2 replicates each.

Intrasplenic transplantation of hPSC-derived hepatocytes. Mice were handled according to procedures approved by Stanford's Administrative Panel for Laboratory Animal Care (APLAC). FRG mice on the NOD genetic background (Yecuris) were fed ab libitum with low-protein, high-fat irradiated Lab Diet 5LJ5 and water containing 8 mg/L of NTBC (2-(2-nitro-4-fluoromethylbenzoyl)-1,3-cyclohexanedione), a hepatoprotective drug. A week before transplantation, liver injury was induced by initially lowering the dose of NTBC to 4 mg/L NTBC for 3 days, followed by withdrawing NTBC for 3-5 days. 0.5-2×10⁶ hPSC-derived hepatocytes (resuspended in 50 μL of media) was injected into the spleen of 6-to 12-week-old FRG mice using 26-31 gauge needles. 6 weeks post-transplantation, FRG mice were sacrificed to analyze the degree of hPSC-derived hepatocyte engraftment in the injured liver.

Glycogen and Triglyceride Assays. 1 million freshly thawed cells were resuspended in PBS and immediately used for the glycogen assay (Abcam, ab169558) or triglyceride assay (ZenBio, TG-1-NC) as per the manufacturers' instructions. Colorimetric readings were performed using the Tecan Infinite 200Pro plate reader. Normalization was performed by subtracting background noise per the manufacturers' instructions and, when appropriate, cell sample volume input.

Cell Viability Assay. Cell viability assay was performed using the LIVE/DEAD viability/cytotoxicity kit (Thermo Fisher, L3224). Cells were washed once with DMEM/F12 media and then stained with 1 μM Calcein-AM+3 μM EthD-1 in DMEM/F12 media at room temperature for 30 minutes in the dark. The cells were then gently washed once with DMEM/F12 media, before imaging on an Olympus FV3000 confocal microscope.

Induction of lipid droplets in hPSC-derived hepatocytes. hPSCs were first differentiated into day 6 liver bud progenitors as described above (Ang et al., 2018). They were then differentiated for 12 additional days into hepatocytes, largely using the differentiation-inducing signals described above, but in lieu of CDM4B basal medium, the lipid- and glucose-rich LipidHep basal medium (composition above) was used instead.

On days 7-12, the following signals were added to induce early hepatocytes: Forskolin (10 μM), Dexamethasone (10 μM, Tocris), RO4929097 (2 μM, Cellagen), AA2P (200 μg/mL, Cayman), Insulin (10 μg/mL, Sigma), BMP4 (10 ng/ml), and OSM (10 ng/ml, R&D Systems) for 6 days in LipidHep basal medium. Day 12 early hepatocytes were then briefly washed (DMEM/F12) and then differentiated in CDM4B media supplemented with Forskolin (10 μM), Dexamethasone (10 M), RO4929097 (2 μM), AA2P (200 μg/mL), and Insulin (10 μg/mL) for 6 days in LipidHep basal medium, thereby yielding day 18 hepatocytes.

Staining was performed with Oil Red O staining solution (Lifeline Cell Technology) following the manufacturer's instructions. First, cells were fixed with 4% paraformaldehyde for 20 minutes at room temperature. Next, cells were washed twice with deionized water before adding 1,2-Propanediol dehydration solution. Cells were dehydrated at room temperature for 5 minutes. After dehydration, Oil Red O stain solution (Lifeline Technology) was added to the cells and left at 37° C. for 30 minutes, after which Oil Red O dye was removed and 1,2-Propanediol stain differential solution was added to cells to differentiate the stain. Differential solution was removed after 1 minute and cells were washed twice with deionized water before imaging.

Poly(I:C) treatment. HepG2 cells, HuH7 cells, metabolically-selected H1 hPSC-and H7 hPSC-derived hepatocytes, and primary human hepatocytes (Lonza and Gibco) were thawed in a 37° C. water bath for 3 minutes. After thawing, HepG2, HuH7, and hPSC-derived hepatocytes were quenched in warm DMEM/F12 to dilute the cryopreservation solution. After thawing, primary human hepatocytes, obtained from either Lonza or Gibco, were respectively quenched in MCHT50 and CHRM to dilute the cryopreservation solution. Thawed cells were centrifuged at 1500 rpm for 5 mins and the supernatant was discarded, leaving the cell pellets. The cell pellets were resuspended in the aforementioned hepatocyte maintenance media (Lonza hepatocyte culture medium supplemented with 10 μM Dexamethasone, 10 μM Forskolin, 2 μM RO4929097, 200 μg/ml AA2P, 10 μg/ml Insulin, and 1 μM Thiazovivin [DFRAIT]). For each cell-type, ~400,000 cells were plated per well of 24-well plate that had been coated with collagen 1 (Corning Biocoat). For hPSC-derived hepatocytes, the collagen 1-coated wells were briefly treated with Geltrex prior to plating cells. The media was replenished every day for 2 days and on the third day, hepatocyte maintenance media+ DFRAIT+50 μg/ml poly(I:C) was added for 24 hours. Cells were then lysed with RNA lysis buffer (Zymo) and processed with an RNA extraction kit (Zymo) to extract RNA for qPCR and RNA-seq.

Generating stocks of Ebola, Sudan, Marburg, and Lassa viruses and viral quantification. Authentic Ebola, Sudan, Marburg, and Lassa viruses are classified in Risk Group 4 (CDC and NIH, 2020). All experiments with Risk Group 4 viruses were conducted under maximum containment conditions in the biosafety level 4 (BSL4) laboratory of the Robert Koch Institute, in accordance with standard operating procedures institutionally approved by the Robert Koch Institute. Work at the Robert Koch Institute was performed using only terminally-differentiated hepatocytes.

Wild-type Ebola virus (Johnson et al., 1977), wild-type Lassa virus (Callis et al., 1982), GFP-expressing Ebola virus (Hoenen et al., 2013), zsGreen-expressing Sudan virus (Kainulainen et al., 2023), zsGreen-expressing Marburg virus (Albarino et al., 2018), and zsGreen-expressing Lassa virus (Welch et al., 2016) were obtained from NIH and CDC reference stocks, as described above. These viruses were then subsequently propagated on Vero E6 cells (Vero C1008 (Vero 76, clone E6, Vero E6), from the European Collection of Authenticated Cell Cultures (ECACC)) in DMEM+2% FBS to generate archive stocks, which were frozen at −80° C. As needed, archive stocks were thawed and passaged again on Vero E6 to generate working stocks of these viruses, which were frozen at −80° C. Working stocks of these viruses were used for all subsequent experiments.

To titrate viral stocks, viral quantitation was performed using the fluorescent focus-forming unit (FFU) assay. In brief, Vero E6 cells were seeded into 96-well tissue culture plates in DMEM containing 10% FBS, 2 mM glutamine and 1% penicillin/streptomycin, at such a density that after overnight incubation at 37° C. and 5% CO₂, the cells were 95-100% confluent. Separately, virus stocks were diluted in DMEM in serial 10-fold dilutions. Then, the Vero E6 culture media was aspirated, and 100 μL of viral supernatant (containing various serial dilutions of the original viral stock) was added to triplicate wells of Vero E6 cells. After incubation for 1 hour, viral supernatant was removed and media comprising DMEM+2% FBS+1% carboxymethylcellulose (CMC) was added. After 3-4 days, foci of infected cells were quantified using the two following strategies.

For zsGreen-and GFP-expressing viruses, fluorescent foci of infected cells were directly enumerated and the titer/mL of the input viral supernatant was calculated.

For wild-type viruses, foci of infected cells were detected by antibody staining. In brief, the CMC overlay was removed and cells were fixed in 10% formalin for 10 minutes, permeabilized in DPBS+0.1% Triton X-100 at room temperature for 5 minutes, washed 1× in DPBS, and then blocking buffer containing 2% BSA was incubated for 1 hour at 37° C. Subsequently, 100 μL of primary antibody (anti-Ebola NP, clone 173/303/109, diluted 1:2500, or alternatively, anti-Lassa NP, clone EBS-I-306, Progen, 691652, diluted 1:8000) diluted in 1% BSA was used to stain the cells for 1 hour at 37° C. Cells were then washed and then incubated with 100 μL of secondary antibody (goat anti-mouse IgG Alexa 488, Jackson ImmunoResearch, #115-545-003, diluted 1:500-1:1000) for 30 minutes at 37° C., washed once, and then foci were enumerated. Finally, the titer/mL of the input viral supernatant was calculated.

Generation of concentrated and purified Ebola and Lassa virus stocks. Stocks of wild-type Ebola and Lassa viruses were highly concentrated prior to using them to infect hPSC-derived hepatocytes. 270 mL of virus stock solution was concentrated by multiple rounds of centrifugation at 3000 rpm using Amicon Ultra-15 Centrifugal Filter Units (Merck, UFC9100), followed by a wash with a tenfold excess volume of DMEM, and then the addition of FBS to a final concentration of 10%

Titration of viral stocks on hPSC-derived hepatocytes. Concentrated viral stocks were titrated on both Vero E6 cells and hPSC-derived hepatocytes using the FFU assay, to determine the relative susceptibility of these two cell-types to the same amount of input virus.

7×10⁴ Vero E6 cells or 4.8×10⁵ hPSC-derived hepatocytes were plated onto 20 wells of a 48-well plate that had been previously coated with Geltrex. Vero E6 cells were cultured in DMEM+2% FBS+2 mM glutamine+1% penicillin/streptomycin, while hPSC-derived hepatocytes were cultured in hepatocyte maintenance medium (Lonza HCM supplemented with 10 μM Dexamethasone, 10 μM Forskolin, 2 μM RO4929097, 200 μg/ml AA2P, 10 μg/ml Insulin, and 2 μM Thiazovivin [DFRAIT]). The media was changed daily, and 3 days later, the cells were 100% confluent. Culture media was replaced with 100 μL of medium containing 50, 100, 200, 400, or 2000 focus-forming units (FFU, as previously quantified using Vero E6 cells) of virus. Cells were incubated with virus for 1 hour at 37° C. After 1 hour, the viral inoculum was replaced with 450 μL hepatocyte culture medium mixed with 3:1 with 3% carboxymethylcellulose (CMC) in water (such that the final concentration of CMC was 1%). Three days post-inoculation, images were acquired with an EVOS-FL microscope, and fluorescent foci of infected cells were enumerated to calculate the infectious units of the original viral stock.

Infection of hPSC-derived hepatocytes with fluorescent reporter Risk Group 4 viruses. Metabolically-selected H7 hPSC-derived hepatocytes were thawed and 370,000 hepatocytes were plated per well of 48-well plates that had been previously coated with Geltrex. As described above, hPSC-derived hepatocytes were cultured in hepatocyte maintenance medium (Lonza HCM supplemented with 10 μM Dexamethasone, 10 μM Forskolin, 2 μM RO4929097, 200 μg/ml AA2P, 10 μg/ml Insulin, and 2 μM Thiazovivin [DFRAIT]). One day after thawing, hepatocyte medium was replaced with 100 μL of virus inoculum containing 10e5, 10e3, or 10e2 FFU (FFU calculated based on titration on Vero E6 cells) of GFP-expressing Ebola virus, zsGreen-expressing Sudan virus, zsGreen-expressing Marburg virus, or zsGreen-expressing Lassa virus. At 1, 2, 3, 5, and 7 days post-infection (dpi), images were acquired to visualize the extent of infected hepatocytes, and culture media (supernatants) was collected to quantify the number of extracellular viral particles.

Immunostaining of hPSC-derived hepatocytes infected by Risk Group 4 viruses. For immunostaining experiments, cells were initially seeded onto Geltrex-coated glass cover slips. When the experiment was terminated, culture media was aspirated from virally-infected cells, cells were rinsed once with PBS, and then cells were inactivated according to institutionally-approved standard operating procedures. In brief, cells were fixed in 10% formalin for 10 minutes inside the biosafety cabinet under BSL4 containment, followed by an overnight incubation at 4° C. with 10% formalin (Roti® Histofix 10%); they were then removed from the BSL4 laboratory on the next day. After fixation, the cells were washed four times with 1×PBS. Next, the formaldehyde was quenched using 0.3 M glycine in PBS (pH 7.4). Then, the cells were permeabilized and blocked using a permeabilization/blocking buffer (10% donkey serum+0.1% Triton X100 in 1×PBS) for 1 hour at room temperature. Next, the cells were stained with primary antibodies in 1% donkey serum containing 0.1% Triton X100 in 1×PBS overnight at 4° C. The next day, the cells were washed three times with 0.1% TritonX100 in 1×PBS. Afterwards, the cells were stained with fluorophore-conjugated secondary antibodies diluted in 1% donkey serum+0.1% Triton X100 in 1×PBS in the dark for one hour at room temperature. Finally, the cells were washed four times with 0.1% Triton X100 in 1×PBS and rinsed quickly with MilliQ H₂O. 4 μL Roti Mount HP20.1 Fluor Care DAPI was pipetted onto a glass slide and the coverslip was inverted on this drop of mounting medium. The coverslip was then sealed with nail polish. The images were acquired using a confocal microscope (Zeiss LSM780) and analyzed using Zeiss Zen (black edition).

Bulk population RNA-seq of hPSC-derived hepatocytes with Risk Group 4 viruses. Infection with Risk Group 4 viruses was performed in a biosafety cabinet under BSL4 containment. Metabolically-selected hPSC-derived hepatocytes were inoculated with wild-type Ebola virus, Mayinga isolate (multiplicity of infection [MOI]≈5), wild-type Lassa virus, Josiah isolate (MOI≈5), Sendai virus, Cantell strain (50 HA units), or were mock infected (negative control) in triplicate. Viral infection was performed for 1 hour in a 37° C. incubator. One hour after infection, the viral inoculum was replaced with hepatocyte maintenance medium (described above), and cells were incubated at 37° C. At 6 hours post-infection, or 1, 2, 3, 5, 7 days post-infection (dpi), the culture media (supernatants) was collected and the cells were lysed in 350 μL RLT buffer per well (RNeasy Micro kit; Qiagen, 74004) containing 1:100 2-mercaptoethanol. Lysed cells in RLT buffer were transferred into 600 μL 70% ethanol to inactivate infectious material, as per an institutionally-approved standard operating procedure for viral inactivation, followed by removal out of the BSL4 laboratory. At each harvest time point, with the exception of 6 hours post-infection, the hepatocyte maintenance medium was refreshed on all remaining plates.

RNA was extracted using the RNeasy Micro Kit (Qiagen) as per the manufacturer's instructions. After quality control procedures, polyadenylated mRNA was enriched using oligo (dT) beads. The mRNA was then fragmented randomly in fragmentation buffer, followed by cDNA synthesis using random hexamers and reverse transcriptase. After first-strand synthesis, a second-strand synthesis buffer (Illumina) was added with dNTPs, RNase H and E. coli polymerase I to generate the second strand by nick-translation. The final cDNA library was then subjected to a round of purification, terminal repair, A-tailing, ligation of sequencing adapters, size selection and PCR enrichment. RNA-seq libraries were sequenced on an Illumina NovaSeq 6000 S4 sequencer.

After obtaining RNA-seq reads, quality control was performed using FastQC (Andrews, 2010) and Trim Galore (Krueger, 2012) as described above. Trimmed and filtered reads from each individual library (which was originally sequenced on multiple lanes) were then merged into a single pooled file for each library. RNA-seq profiles of virus-infected cells contained both human and viral reads. To analyze human (host cell) transcripts, reads were aligned to the GRCh38 (hg38) Homo sapiens reference genome, and gene-level RNA-seq counts were quantified using Kallisto (Bray et al., 2016). These counts were imported into RStudio and differential gene expression analyses of these counts were performed using DESeq2 (Love et al., 2014). For all samples, regularized log transformation was performed on the counts to remove the dependence of variance on the mean.

To determine the overall percentage of viral reads within a given RNA-seq library, Kallisto (Bray et al., 2016) was used to align reads to the reference genomes of Ebola virus, Mayinga isolate (NCBI accession number NC_002549.1) or Sendai virus (NCBI accession number NC_001552). This enabled the detection of all viral reads within a given sample. Then the number of total viral counts was divided over the number of total human+ viral counts to estimate what proportion of the cellular transcriptome comprised viral transcripts. Viral read alignment to the Lassa virus genome was not performed, as we performed RNA-seq of polyadenylated mRNAs and therefore did not expect to capture Lassa virus mRNAs, which are not polyadenylated.

Quantification of IFNβ secretion by virally-infected cells. Supernatant (culture media) from virally-infected cells was harvested and then stored at −80° C. Media was then thawed, and the concentration of secreted interferon-β (IFNβ) protein in the undiluted media was quantified using ELISA (enzyme-linked immunosorbent assay), specifically using the Human IFNβ Quantikine Quickit ELISA Kit (R&D Systems, QK410) following the manufacturer's instructions and as described previously (Ang et al., 2022). Absorbance was quantified using a TECAN Sunrise plate reader (Tecan Trading AG, Switzerland) with a spectral filter. The concentration of IFNβ in the samples was calculated by comparing the absorbance to that of a standard curve generated with human IFNβ, after subtracting background absorbance, using TECAN Magellan software. Data shown represent the average results from three well replicates.

Quantification of cell death after viral infection. To quantify the degree of cell death, the supernatant (culture media) from virally-infected cells was harvested and subject to an adenylate kinase assay. In brief, cell death leads to the release of intracellular adenylate kinase into the culture media, which can be enzymatically assayed. Culture media was first inactivated by dilution at a ratio of 4:1 in Triton X-100 and NP40, to a final concentration of 0.5% for each detergent, followed by heating to 60° C. for 10 minutes. Media then removed from BSL4 containment, and a luminescent adenylate kinase assay kit (Abcam, ab228557) was used as per the manufacturer's instructions.

Infection of non-human primates with Ebola virus and Lassa virus. No living non-human primates (NHPs) were assessed as part of this study. In this work, we performed staining of archived livers from rhesus macaques (Macaca mulatta) that had been previously infected with Ebola virus (Bushmaker et al., 2023) and cynomolgus macaques (Macaca fascicularis) that had been previously infected with Lassa virus (Rosenke et al., 2018). These primates have been described in previous publications (Bushmaker et al., 2023; Rosenke et al., 2018).

In brief, all infectious NHP work and sample inactivation was performed in the maximum containment laboratory at the Rocky Mountain Laboratories (RML), National Institute of Allergy and Infectious Diseases, NIH (located in Hamilton, Montana), applying standard operating protocols approved by the Institutional Biosafety Committee. The NHP studies were approved by the Animal Care and Use Committee (ACUC) at RML. They were performed in strict accordance with the Guide for the Care and Use of Laboratory Animals, Office of Laboratory Animal Welfare, NIH and the Animal Welfare Act, United States Department of Agriculture in an AAALAC International accredited facility. Extensive effort was made to promote the wellbeing of NHPs in these studies in accordance with recommendations from the Weatherall Report. Each NHP was singly housed but within proximity of conspecifics to allow for social interactions. The room was environmentally controlled for humidity, temperature, and light (12-hour light/12-hour dark cycles). Commercial monkey biscuits were fed twice daily, and water was available ad libitum throughout the experiment. The NHPs received a variety of produce, treats, visual, and sensory enrichment throughout the duration of the experiment. NHPs were visually monitored in-person at least twice daily with increased monitoring during the severe stage of the disease. If humane endpoint criteria were reached (>35 on clinical score sheet approved by the RML ACUC), the NHP was humanely euthanized.

Briefly, rhesus macaques (Macaca mulatta) were injected intramuscularly with a lethal dose (10 TCID₅₀ units) of Ebola virus, Kikwit isolate (Ebola virus/Human/COD/1995/Kikwit-9510621) (Bushmaker et al., 2023). 6-8 days post-infection, primates reached the humane endpoint and were euthanized and necropsied (Bushmaker et al., 2023). Cynomolgus macaques (Macaca fascicularis) were injected intramuscularly with a lethal dose (10⁴ TCID₅₀ units) of Lassa virus, Josiah isolate (Lassa virus/Mouse/Sierra Leone/1976/Josiah) (Rosenke et al., 2018). 11 days post-infection, a primate that reached the humane endpoint was euthanized and necropsied (Rosenke et al., 2018). Archived liver tissues from both of these previous studies (Bushmaker et al., 2023; Rosenke et al., 2018) were stained in the present work.

Histology and immunohistochemistry of non-human primate tissues. Non-human primate tissues were fixed in 10% neutral-buffered formalin, removed from the BSL4 laboratory according to approved standard operating procedures, processed, and embedded in paraffin. Slides were cut into 5 μm sections and stained with hematoxylin-eosin for preliminary histologic analysis. For immunohistochemical analysis, primary incubation with HepPar1 antibody (Novus), Ebola virus VP40 antibody (gift of Yoshihiro Kawaoka), and Lassa virus NP antibody (Cusabio) was performed for 1 hour and washed with Tris-based Reaction Buffer (Roche Tissue Diagnostics, 950-300) using the Ventana Discovery Ultra machine. This was followed by secondary antibody incubation with either ImmPRESS-AP horse anti-mouse polymer or ImmPRESS-VP horse anti-rabbit polymer (both from Vector Laboratories; MP-5402 and MP-6401, respectively) for 32 minutes according to manufacturer's instructions. Slides were counter stained with Gill's hematoxylin (Roche Tissue Diagnostics, 760-2021). Imaging was performed by a board-certified veterinary pathologist using an Olympus BX51 microscope with a mounted Olympus DP80 imaging camera.

Viral nomenclature and case fatality rates. International Committee on Taxonomy of Viruses (ICTV)-ratified nomenclature was used to refer to wild-type filoviruses (Kuhn, 2017; Kuhn et al., 2014a) and arenaviruses. A published nomenclature scheme (Kuhn et al., 2014b; Kuhn et al., 2013) was used to refer to recombinant filoviruses, and similar terminology was applied to recombinant arenaviruses.

Estimated case fatality rates for the viruses studied in this manuscript are as follows: Ebola virus: 14,742 deaths among 33,604 cases, ~43.8% case fatality rate (Jacob et al., 2020); Sudan virus: ~53.7% (Jacob et al., 2020); Marburg virus: 478 deaths among 590 cases, ~81.0% case fatality rate; Lassa virus: estimated at ~1% (Prescott et al., 2017)

### REFERENCES

1. CDC, NIH, Biosafety in Microbiological and Biomedical Laboratories—6th Edition. (U.S. Department of Health and Human Services, Atlanta, GA, 2020), pp. 1-604.
2. S. Kuthyar, C. L. Anthony, T. Fashina, S. Yeh, J. G. Shantha, World Health Organization High Priority Pathogens: Ophthalmic Disease Findings and Vision Health Perspectives. Pathogens 10, 442 (2021).
3. J.-A. Røttingen et al., New Vaccines against Epidemic Infectious Diseases. The New England Journal of Medicine 376, 610-613 (2017).
4. J. Kuhn, Filoviruses: A Compendium of 40 Years of Epidemiological, Clinical, and Laboratory Studies. Archives of virology. Supplement 20. (Springer Vienna: Imprint: Springer, Vienna, ed. 1st 2008., 2008).
5. H. Feldmann, A. Sprecher, T. W. Geisbert, Ebola. N Engl J Med 382, 1832-1842 (2020).
6. H. Feldmann, T. W. Geisbert, Ebola haemorrhagic fever. Lancet 377, 849-862 (2011).
7 S. T. Jacob et al., Ebola virus disease. Nat Rev Dis Primers 6, 13 (2020).
8. E. I. Ohimain, D. Silas-Olu, The 2013-2016 Ebola virus disease outbreak in West Africa. Curr Opin Pharmacol 60, 360-365 (2021).
9 D. Gatherer, The 2014 Ebola virus disease outbreak in West Africa. J. Gen. Virol. 95, 1619-1624 (2014).
10. M. G. Dixon, I. J. Schafer, C. Centers for Disease, Prevention, Ebola viral disease outbreak—West Africa, 2014. MMWR Morb Mortal Wkly Rep 63, 548-551 (2014).
11. T. Kiggundu et al., Notes from the Field: Outbreak of Ebola Virus Disease Caused by Sudan ebolavirus—Uganda, August-October 2022. MMWR Morb Mortal Wkly Rep 71, 1457-1459 (2022).
12. P. Musoke, F. Bongomin, Sudan virus disease outbreak in Uganda in 2022: the case of patient zero. Int J Infect Dis 128, 318-320 (2023).
13. O. Sibomana, E. Kubwimana, First-ever Marburg virus disease outbreak in Equatorial Guinea and Tanzania: An imminent crisis in West and East Africa. Immun Inflamm Dis 11, e980 (2023).
14. J. B. Prescott et al., Immunobiology of Ebola and Lassa virus infections. Nature Reviews Immunology 17, 195-207 (2017).
15. S. Gunther, O. Lenz, Lassa virus. Crit Rev Clin Lab Sci 41, 339-390 (2004).
16. K. G. Andersen et al., Clinical Sequencing Uncovers Origins and Evolution of Lassa Virus. Cell 162, 738-750 (2015).
17. I. S. Caballero et al., Lassa and Marburg viruses elicit distinct host transcriptional responses early after infection. BMC Genomics 15, 960 (2014).
18. J. v. Paassen et al., Acute liver failure, multiorgan failure, cerebral oedema, and activation of proangiogenic and antiangiogenic factors in a case of Marburg haemorrhagic fever. The Lancet Infectious Diseases 12, 635-642 (2012).
19. T. W. Geisbert et al., Marburg Virus Angola Infection of Rhesus Macaques: Pathogenesis and Treatment with Recombinant Nematode Anticoagulant Protein c2. The Journal of Infectious Diseases 196, S372-S381 (2007).
20. J. B. McCormick et al., Lassa Virus Hepatitis: a Study of Fatal Lassa Fever in Humans. Am J Tropical Medicine Hyg 35, 401-407 (1986).
21. R. B. Martines, D. L. Ng, P. W. Greer, P. E. Rollin, S. R. Zaki, Tissue and cellular tropism, pathology and pathogenesis of Ebola and Marburg viruses. The Journal of Pathology 235, 153-174 (2014).
22. P. E. Rollin, D. G. Bausch, A. Sanchez, Blood Chemistry Measurements and d-Dimer Levels Associated with Fatal and Nonfatal Outcomes in Humans Infected with Sudan Ebola Virus. The Journal of Infectious Diseases 196, S364-S371 (2007).
23. K. M. Johnson, J. V. Lange, P. A. Webb, F. A. Murphy, Isolation and Partial Characterisation of a New Virus causing Acute Haemorrhagic Fever in Zaire. The Lancet 309, 569-571 (1977).
24. L. E. Hensley et al., Pathogenesis of lassa fever in cynomolgus macaques. Virol J 8, 205 (2011).
25. W.-J. Shieh et al., Pathology and Pathogenesis of Lassa Fever: Novel Immunohistochemical Findings in Fatal Cases and Clinico-pathologic Correlation. Clin. Infect. Dis. 74, ciab719-(2021).
26. D. H. Walker et al., Pathologic and virologic study of fatal Lassa fever in man. Am J Pathol 107, 349-356 (1982).
27. H. Bechtelsheimer, G. Korb, P. Gedigk, The morphology and pathogenesis of “Marburg virus” hepatitis. Hum Pathol 3, 255-264 (1972).
28. D. S. Ellis, E. T. Bowen, D. I. Simpson, S. Stamford, Ebola virus: a comparison, at ultrastructural level, of the behaviour of the Sudan and Zaire strains in monkeys. Br J Exp Pathol 59, 584-593 (1978).
29. D. S. Ellis et al., Ultrastructure of Ebola virus particles in human liver. J Clin Pathol 31, 201-208 (1978).
30. A. Greenberg et al., Quantification of Viral and Host Biomarkers in the Liver of Rhesus Macaques A Longitudinal Study of Zaire Ebolavirus Strain Kikwit (EBOV/Kik). The American Journal of Pathology 190, 1449-1460 (2020).
31. M. A. Vernet et al., Clinical, virological, and biological parameters associated with outcomes of Ebola virus infection in Macenta, Guinea. JCI Insight 2, e88864 (2017).
32. R. W. Cross et al., Natural history of nonhuman primates after conjunctival exposure to Ebola virus. Sci Rep 13, 4175 (2023).
33. T. W. Geisbert, L. E. Hensley, Ebola virus: new insights into disease aetiopathology and possible therapeutic interventions. Expert Rev Mol Med 6, 1-24 (2004).
34. A. Jankeel et al., Early Transcriptional Changes within Liver, Adrenal Gland, and Lymphoid Tissues Significantly Contribute to Ebola Virus Pathogenesis in Cynomolgus Macaques. J. Virol. 94, (2020).
35. C. Woolsey et al., Natural history of Sudan ebolavirus infection in rhesus and cynomolgus macaques. Emerg Microbes Infect 11, 1635-1646 (2022).
36. R. Carrion, Jr. et al., Lassa virus infection in experimentally infected marmosets: liver pathology and immunophenotypic alterations in target tissues. J. Virol. 81, 6482-6490 (2007).
37. A. C. Shurtleff et al., The impact of regulations, safety considerations and physical limitations on research progress at maximum biocontainment. Viruses 4, 3932-3951 (2012).
38. A. L. Hartman, J. E. Dover, J. S. Towner, S. T. Nichol, Reverse Genetic Generation of Recombinant Zaire Ebola Viruses Containing Disrupted IRF-3 Inhibitory Domains Results in Attenuated Virus Growth In Vitro and Higher Levels of IRF-3 Activation without Inhibiting Viral Transcription or Replication. J. Virol. 80, 6430-6440 (2006).
39. A. L. Hartman, L. Ling, S. T. Nichol, M. L. Hibberd, Whole-Genome Expression Profiling Reveals That Inhibition of Host Innate Immune Response Pathways by Ebola Virus Can Be Reversed by a Single Amino Acid Change in the VP35 Protein. J. Virol. 82, 5348-5358 (2008).
40. J. Logue et al., Ebola Virus Isolation Using Huh-7 Cells has Methodological Advantages and Similar Sensitivity to Isolation Using Other Cell Types and Suckling BALB/c Laboratory Mice. Viruses 11, 161 (2019).
41. I. V. Kuzmin et al., Innate Immune Responses of Bat and Human Cells to Filoviruses: Commonalities and Distinctions. J Virol 91, (2017).
42. M. Flint et al., A genome-wide CRISPR screen identifies N-acetylglucosamine-1-phosphate transferase as a potential antiviral target for Ebola virus. Nat Commun 10, 285 (2019).
43. S. R. Welch et al., Lassa and Ebola virus inhibitors identified using minigenome and recombinant virus reporter systems. Antiviral Res 136, 9-18 (2016).
44. B. Zhou et al., Haplotype-resolved and integrated genome analysis of the cancer cell line HepG2. Nucleic Acids Research 47, gkz169-(2019).
45. F. Kasai, N. Hirayama, M. Ozawa, M. Satoh, A. Kohara, HuH-7 reference genome profile: complex karyotype composed of massive loss of heterozygosity. Hum Cell 31, 261-267 (2018).
46. S. Marozin et al., Inhibition of the IFN-β Response in Hepatocellular Carcinoma by Alternative Spliced Isoform of IFN Regulatory Factor-3. Molecular Therapy 16, 1789-1797 (2008).
47. K. Li, Z. Chen, N. Kato, M. Gale, S. M. Lemon, Distinct Poly (I-C) and Virus-activated Signaling Pathways Leading to Interferon-β Production in Hepatocytes*. Journal of Biological Chemistry 280, 16739-16747 (2005).
48. W. A. Scoon et al., Ebola virus infection induces a delayed type I IFN response in bystander cells and the shutdown of key liver genes in human iPSC-derived hepatocytes. Stem Cell Reports, (2022).
49. J. P. Cotovio, T. G. Fernandes, Production of Human Pluripotent Stem Cell-Derived Hepatic Cell Lineages and Liver Organoids: Current Status and Potential Applications. Bioeng 7, 36 (2020).
50. S. W. Maepa, H. Ndlovu, Advances in generating liver cells from pluripotent stem cells as a tool for modeling liver diseases. Stem Cells 38, 606-612 (2020).
51. T. S. Tracy et al., Interindividual Variability in Cytochrome P450-Mediated Drug Metabolism. Drug Metabolism and Disposition 44, 343-351 (2016).
52. K. A. Telles-Silva et al., Applied Hepatic Bioengineering: Modeling the Human Liver Using Organoid and Liver-on-a-Chip Technologies. Frontiers Bioeng Biotechnology 10, 845360 (2022).
53. A. I. Ardisasmita et al., A comprehensive transcriptomic comparison of hepatocyte model systems improves selection of models for experimental use. Commun Biol 5, 1094 (2022).
54. J. L. Fowler, L. T. Ang, K. M. Loh, A critical look: Challenges in differentiating human pluripotent stem cells into desired cell types and organoids. Wiley Interdisciplinary Reviews: Developmental Biology 113, 891-823 (2019).
55. J. A. Thomson et al., Embryonic Stem Cell Lines Derived from Human Blastocysts. Science 282, 1145-1147 (1998).
56. K. Takahashi et al., Induction of Pluripotent Stem Cells from Adult Human Fibroblasts by Defined Factors. Cell 131, 861-872 (2007).
57. A. K. Y. Tan, K. M. Loh, L. T. Ang, Evaluating the regenerative potential and functionality of human liver cells in mice. Differentiation 98, 25-34 (2017).
58. M. E. Levenstein et al., Basic fibroblast growth factor support of human embryonic stem cell self-renewal. Stem Cells 24, 568-574 (2006).
59. T. Takebe et al., Vascularized and functional human liver from an iPSC-derived organ bud transplant. Nature 499, 481-484 (2013).
60. S. Ogawa et al., Three-dimensional culture and cAMP signaling promote the maturation of human pluripotent stem cell-derived hepatocytes. Development 140, 3285-3296 (2013).
61. A. Carpentier et al., Engrafted human stem cell-derived hepatocytes establish an infectious HCV murine model. Journal of Clinical Investigation 124, 4953-4964 (2014).
62. K. Si Tayeb et al., Highly efficient generation of human hepatocyte-like cells from induced pluripotent stem cells. Hepatology 51, 297-305 (2010).
63. D. Zhao et al., Promotion of the efficient metabolic maturation of human pluripotent stem cell-derived hepatocytes by correcting specification defects. Cell Research 23, 157-161 (2012).
64. T. Touboul et al., Generation of functional hepatocytes from human embryonic stem cells under chemically defined conditions that recapitulate liver development. Hepatology 51, 1754-1765 (2010).
65. T. Sumi, N. Tsuneyoshi, N. Nakatsuji, H. Suemori, Defining early lineage specification of human embryonic stem cells by the orchestrated balance of canonical Wnt/-catenin, Activin/Nodal and BMP signaling. Development 135, 2969-2979 (2008).
66. A. Yanagida, K. Ito, H. Chikada, H. Nakauchi, A. Kamiya, An In Vitro Expansion System for Generation of Human iPS Cell-Derived Hepatic Progenitor-Like Cells Exhibiting a Bipotent Differentiation Potential. PLoS ONE 8, e67541 (2013).
67. P. Gadue, T. L. Huber, P. J. Paddison, G. M. Keller, Wnt and TGF-β signaling are required for the induction of an in vitro model of primitive streak formation using embryonic stem cells. Proceedings of the National Academy of Sciences 103, 16806-16811 (2006).
68. V. Gouon-Evans et al., BMP-4 is required for hepatic specification of mouse embryonic stem cell-derived definitive endoderm. Nature Biotechnology 24, 1402-1411 (2006).
69. L. T. Ang et al., A Roadmap for Human Liver Differentiation from Pluripotent Stem Cells. Cell Reports 22, 2190-2205 (2018).
70. J. Cai et al., Directed differentiation of human embryonic stem cells into functional hepatic cells. Hepatology 45, 1229-1239 (2007).
71. S. Agarwal, K. L. Holton, R. Lanza, Efficient Differentiation of Functional Hepatocytes from Human Embryonic Stem Cells. Stem Cells 26, 1117-1127 (2008).
72. D. Haridass et al., Repopulation Efficiencies of Adult Hepatocytes, Fetal Liver Progenitor Cells, and Embryonic Stem Cell-Derived Hepatic Cells in Albumin-Promoter-Enhancer Urokinase-Type Plasminogen Activator Mice. The American Journal of Pathology 175, 1483-1492 (2009).
73. S. Han, Generation of Functional Hepatic Cells from Pluripotent Stem Cells. Journal of Stem Cell Research and Therapy 1, 1-7 (2012).
74. Z. Song et al., Efficient generation of hepatocyte-like cells from human induced pluripotent stem cells. Cell Research 19, 1233-1242 (2009).
75. N. Shiraki et al., Differentiation of mouse and human embryonic stem cells into hepatic lineages. Genes to Cells 13, 731-746 (2008).
76. Y. Avior et al., Microbial-derived lithocholic acid and vitamin K2 drive the metabolic maturation of pluripotent stem cells-derived and fetal hepatocytes. Hepatology 62, 265-278 (2015).
77. A. Carpentier et al., Hepatic differentiation of human pluripotent stem cells in miniaturized format suitable for high-throughput screen. Stem Cell Research 16, 640-650 (2016).
78. L. Tolosa et al., Transplantation of hESC-derived hepatocytes protects mice from liver injury. Stem Cell Research & Therapy 6, 246 (2015).
79. Y. Nagamoto et al., Transplantation of a human iPSC-derived hepatocyte sheet increases survival in mice with acute liver failure. Journal of Hepatology 64, 1068-1075 (2016).
80. H. Ma et al., The nuclear receptor THRB facilitates differentiation of human PSCs into more mature hepatocytes. Cell Stem Cell 29, 795-809 e711 (2022).
81. K. M. Loh et al., Efficient Endoderm Induction from Human Pluripotent Stem Cells by Logically Directing Signals Controlling Lineage Bifurcations. Cell Stem Cell 14, 237-252 (2014).
82. S. Tohyama, S. Tanosaki, S. Someya, J. Fujita, K. Fukuda, Manipulation of Pluripotent Stem Cell Metabolism for Clinical Application. Curr Stem Cell Reports 3, 28-34 (2017).
83. S. Tohyama et al., Distinct Metabolic Flow Enables Large-Scale Purification of Mouse and Human Pluripotent Stem Cell-Derived Cardiomyocytes. Cell Stem Cell 12, 127-137 (2013).
84. B. Lin et al., Culture in Glucose-Depleted Medium Supplemented with Fatty Acid and 3,3′, 5-Triiodo-I-Thyronine Facilitates Purification and Maturation of Human Pluripotent Stem Cell-Derived Cardiomyocytes. Frontiers in endocrinology 8, E1848 (2017).
85. P. B. Alexander, J. Wang, S. L. McKnight, Targeted killing of a mammalian cell based upon its specialized metabolic state. Proc Natl Acad Sci USA 108, 15828-15833 (2011).
86. Y. Taya et al., Depleting dietary valine permits nonmyeloablative mouse hematopoietic stem cell transplantation. Science 354, 1152-1155 (2016).
87. J. Wang et al., Dependence of mouse embryonic stem cells on threonine catabolism Science 325, 435-439 (2009).
88. H.-S. Han, G. Kang, J. S. Kim, B. H. Choi, S.-H. Koo, Regulation of glucose metabolism from a liver-centric perspective. Exp Mol Medicine 48, e218-e218 (2016).
89. L. Rui, Energy Metabolism in the Liver. Compr Physiol 4, 177-197 (2017).
90. M. M. Adeva-Andany, N. Pérez-Felpete, C. Fernández-Fernández, C. Donapetry-García, C. Pazos-García, Liver glucose metabolism in humans. Bioscience Rep 36, e00416 (2016).
91. P. J. Klover, R. A. Mooney, Hepatocytes: critical for glucose homeostasis. Int J Biochem Cell Biology 36, 753-758 (2004).
92. D. T. Peters et al., Asialoglycoprotein receptor 1 is a specific cell-surface marker for isolating hepatocytes derived from human pluripotent stem cells. Development 143, 1475-1481 (2016).
93. G. X. Y. Zheng et al., Massively parallel digital transcriptional profiling of single cells. Nature Communications 8, 14049 (2017).
94. M. R. Corces et al., An improved ATAC-seq protocol reduces background and enables interrogation of frozen tissues. Nature Methods 14, 959-962 (2017).
95. M. Kanai-Azuma et al., Depletion of definitive gut endoderm in Sox17-null mutant mice. Development 129, 2367-2379 (2002).
96. S. Engert, W. P. Liao, I. Burtscher, H. Lickert, Sox17-2A-iCre: A knock-in mouse line expressing Cre recombinase in endoderm and vascular endothelial cells. Genesis 47, 603-610 (2009).
97. S. Engert, I. Burtscher, B. Kalali, M. Gerhard, H. Lickert, The Sox17CreERT2 knock-in mouse line displays spatiotemporal activation of Cre recombinase in distinct Sox17 lineage progenitors. Genesis 51, 793-802 (2013).
98. P. P. L. Tam, D. A. F. Loebel, Gene function in mouse embryogenesis: get set for gastrulation. Nature Reviews Genetics 8, 368-381 (2007).
99. C. C. Lu, E. J. Robertson, J. Brennan, The mouse frizzled 8 receptor is expressed in anterior organizer tissues. Gene Expr Patterns 4, 569-572 (2004).
100. M. Blum et al., Gastrulation in the mouse: the role of the homeobox gene goosecoid. Cell 69, 1097-1106 (1992).
101. W. Shawlot, J. M. Deng, R. R. Behringer, Expression of the mouse cerberus-related gene, Cerr1, suggests a role in anterior neural induction and somitogenesis. Proc Natl Acad Sci USA 95, 6198-6203 (1998).
102. C. Biben et al., Murine cerberus homologue mCer-1: a candidate anterior patterning molecule. Developmental Biology 194, 135-151 (1998).
103. T. H.-W. Ludtke, V. M. Christoffels, M. Petry, A. Kispert, Tbx3 promotes liver bud expansion during mouse development by suppression of cholangiocyte differentiation. Hepatology 49, 969-978 (2009).
104. Y. Guz et al., Expression of murine STF-1, a putative insulin gene transcription factor, in beta cells of pancreas, duodenal epithelium and pancreatic exocrine and endocrine progenitors during ontogeny. Development 121, 11-18 (1995).
105. K. Chawengsaksophak, W. d. Graaff, J. Rossant, J. Deschamps, F. Beck, Cdx2 is essential for axial elongation in mouse development. Proceedings of the National Academy of Sciences 101, 7641-7645 (2004).
106. F. Beck, T. Erler, A. Russell, R. James, Expression of Cdx-2 in the mouse embryo and placenta: possible role in patterning of the extra-embryonic membranes. Dev Dyn 204, 219-227 (1995).
107. A. M. Zorn, J. M. Wells, Vertebrate endoderm development and organ formation. Annu Rev Cell Dev Biol 25, 221-251 (2009).
108. E. Aihara, K. A. Engevik, M. H. Montrose, Trefoil Factor Peptides and Gastrointestinal Function. Annual Review of Physiology 79, 357-380 (2015).
109. Y. Wang et al., Single-cell transcriptome analysis reveals differential nutrient absorption functions in human intestine. J. Exp. Med. 217, e20191130 (2019).
110. R. Asada et al., The Endoplasmic Reticulum Stress Transducer OASIS Is involved in the Terminal Differentiation of Goblet Cells in the Large Intestine*. Journal of Biological Chemistry 287, 8144-8153 (2012).
111. A. Egozi et al., Insulin is expressed by enteroendocrine cells during human fetal development. Nature Medicine 27, 2104-2107 (2021).
112. M. S. Engelstoft, K. L. Egerod, M. L. Lund, T. W. Schwartz, Enteroendocrine cell types revisited. Curr. Opin. Pharm. 13, 912-921 (2013).
113. B. Z. Stanger, R. Datar, L. C. Murtaugh, D. A. Melton, Direct regulation of intestinal fate by Notch. Proceedings of the National Academy of Sciences 102, 12443-12448 (2005).
114. J. Jensen et al., Control of endodermal endocrine development by Hes-1. Nature Genetics 24, 36-44 (2000).
115. J. H. v. Es et al., Notch/γ-secretase inhibition turns proliferative cells in intestinal crypts and adenomas into goblet cells. Nature 435, 959-963 (2005).
116. E. A. Kvittingen, Hereditary tyrosinemia type I—an overview. Scand J Clin Lab Invest Suppl 184, 27-34 (1986).
117. L. Fagerberg et al., Analysis of the Human Tissue-specific Expression by Genome-wide Integration of Transcriptomics and Antibody-based Proteomics. Molecular & Cellular Proteomics 13, 397-406 (2014).
118. N. Aizarani et al., A human liver cell atlas reveals heterogeneity and epithelial progenitors. Nature 572, 1-6 (2019).
119. S. Tohyama et al., Glutamine Oxidation Is Indispensable for Survival of Human Pluripotent Stem Cells. Cell Metabolism 23, 663-674 (2016).
120. M. Tomizawa et al., Survival of Primary Human Hepatocytes and Death of Induced Pluripotent Stem Cells in Media Lacking Glucose and Arginine. PLoS ONE 8, e71897 (2013).
121. T. Zhou et al., The role of PEG3 in the occurrence and prognosis of colon cancer. Onco Targets Ther 12, 6001-6012 (2019).
122. T. Xie et al., PEG10 as an oncogene: expression regulatory mechanisms and role in tumor progression. Cancer Cell Int 18, 112 (2018).
123. C. Tabula Muris et al., Single-cell transcriptomics of 20 mouse organs creates a Tabula Muris. Nature 562, 367-372 (2018).
124. S. B. Naraharisetti et al., Human Liver Expression of CYP2C8: Gender, Age, and Genotype Effects. Drug Metabolism and Disposition 38, 889-893 (2010).
125. S. Michaels, M. Z. Wang, The Revised Human Liver Cytochrome P450 “Pie”: Absolute Protein Quantification of CYP4F and CYP3A Enzymes Using Targeted Quantitative Proteomics. Drug Metabolism and Disposition 42, 1241-1251 (2014).
126. S. N. Hart, Y. Cui, C. D. Klaassen, X. b. Zhong, Three Patterns of Cytochrome P450 Gene Expression during Liver Maturation in Mice. Drug Metabolism and Disposition 37, 116-121 (2008).
127. M. J. Blake, L. Castro, J. S. Leeder, G. L. Kearns, Ontogeny of drug metabolizing enzymes in the neonate. Seminars in Fetal and Neonatal Medicine 10, 123-138 (2005).
128. R. N. Hines, Ontogeny of human hepatic cytochromes P450. J. Biochem. Mol. Toxicol. 21, 169-175 (2007).
129. D. Nitsch, M. Boshart, G. Schutz, Activation of the tyrosine aminotransferase gene is dependent on synergy between liver-specific and hormone-responsive elements. Proc Natl Acad Sci USA 90, 5479-5483 (1993).
130. G. West et al., Key differences between apoC-III regulation and expression in intestine and liver. Biochem. Biophys. Res. Commun. 491, 747-753 (2017).
131. I. A. Haddad, J. M. Ordovas, T. Fitzpatrick, S. K. Karathanasis, Linkage, evolution, and expression of the rat apolipoprotein A-I, C-III, and A-IV genes. J. Biol. Chem. 261, 13268-13277 (1986).
132. K. Jungermann, Zonation of metabolism and gene expression in liver. Histochemistry and Cell Biology 103, 81-91 (1995).
133. R. Gebhardt, Metabolic zonation of the liver: regulation and implications for liver function. Pharmacology and Therapeutics 53, 275-354 (1992).
134. S. Colnot, C. Perret. (Springer US, Boston, MA, 2011), vol. 5, pp. 7-16.
135. N. M. Q. Pek, K. J. Liu, M. Nichane, L. T. Ang, Controversies Surrounding the Origin of Hepatocytes in Adult Livers and the in Vitro Generation or Propagation of Hepatocytes. Cellular and Molecular Gastroenterology and Hepatology, 1-18 (2020).
136. M. Alves-Bezerra, D. E. Cohen, Triglyceride Metabolism in the Liver. Compr Physiol 8, 1-8 (2017).
137. H. Azuma et al., Robust expansion of human hepatocytes in Fah−/−/Rag2−/−/112rg−/− mice. Nature Biotechnology 25, 903-910 (2007).
138. D. D. Smith, J. W. Campbell, Distribution of glutamine synthetase and carbamoyl-phosphate synthetase I in vertebrate liver. Proceedings of the National Academy of Sciences 85, 160-164 (1988).
139. S. Ben-Moshe et al., Spatial sorting enables comprehensive characterization of liver zonation. Nature Metabolism, 1-17 (2019).
140. K. B. Halpern et al., Single-cell spatial reconstruction reveals global division of labour in the mammalian liver. Nature 542, 352-356 (2017).
141. H. M. Lazear, J. W. Schoggins, M. S. Diamond, Shared and Distinct Functions of Type I and Type III Interferons. Immunity 50, 907-923 (2019).
142. L. Alexopoulou, A. C. Holt, R. Medzhitov, R. A. Flavell, Recognition of double-stranded RNA and activation of NF-κB by Toll-like receptor 3. Nature 413, 732-738 (2001).
143. K. Honda et al., Selective contribution of IFN-α/B signaling to the maturation of dendritic cells induced by double-stranded RNA or viral infection. Proceedings of the National Academy of Sciences 100, 10872-10877 (2003).
144. A. E. Shaw et al., Fundamental properties of the mammalian innate immune system revealed by multispecies comparison of type I interferon responses. PLoS Biol. 15, e2004086 (2017).
145. J. Yi et al., Large tumor suppressor homologs 1 and 2 regulate mouse liver progenitor cell proliferation and maturation through antagonism of the coactivators YAP and TAZ. Hepatology 64, 1757-1772 (2016).
146. J. Yamamoto, M. Udono, S. Miura, S. Sekiya, A. Suzuki, Cell Aggregation Culture Induces Functional Differentiation of Induced Hepatocyte-like Cells through Activation of Hippo Signaling. Cell Rep 25, 183-198 (2018).
147. J. H. Driskill, D. Pan, The Hippo Pathway in Liver Homeostasis and Pathophysiology. Annu Rev Pathol 16, 299-322 (2021).
148. P. Sun et al., Maintenance of Primary Hepatocyte Functions In Vitro by Inhibiting Mechanical Tension-Induced YAP Activation. Cell Rep 29, 3212-3222 e3214 (2019).
149. T. Hoenen, A. Groseth, J. Callison, A. Takada, H. Feldmann, A novel Ebola virus expressing luciferase allows for rapid and quantitative testing of antivirals. Antiviral Res 99, 207-213 (2013).
150. M. H. Kainulainen et al., Recombinant Sudan virus and evaluation of humoral cross-reactivity between Ebola and Sudan virus glycoproteins after infection or rVSV-DeltaG-ZEBOV-GP vaccination. Emerg Microbes Infect 12, 2265660 (2023).
151. C. G. Albarino, L. Wiggleton Guerrero, A. K. Chakrabarti, S. T. Nichol, Transcriptional analysis of viral mRNAs reveals common transcription patterns in cells infected by five different filoviruses. PLoS One 13, e0201827 (2018).
152. K. Rosenke et al., Use of Favipiravir to Treat Lassa Virus Infection in Macaques. Emerg Infect Dis 24, 1696-1699 (2018).
153. T. Bushmaker et al., Limited benefit of post-exposure prophylaxis with VSV-EBOV in Ebola virus-infected rhesus macaques. J Infect Dis, (2023).
154. S. L. Butler et al., The antigen for Hep Par 1 antibody is the urea cycle enzyme carbamoyl phosphate synthetase 1. Lab Invest 88, 78-88 (2008).
155. C. B. López, J. S. Yount, T. Hermesh, T. M. Moran, Sendai virus infection induces efficient adaptive immunity independently of type I interferons. Journal of Virology 80, 4538-4545 (2006).
156. S. P. Reid et al., Ebola virus VP24 binds karyopherin alpha1 and blocks STAT1 nuclear accumulation. J. Virol. 80, 5156-5167 (2006).
157. C. F. Basler, Innate immune evasion by filoviruses. Virology 479-480, 122-130 (2015).
158. J. Misasi, N. J. Sullivan, Camouflage and misdirection: the full-on assault of ebola virus disease. Cell 159, 477-486 (2014).
159. I. Messaoudi, G. K. Amarasinghe, C. F. Basler, Filovirus pathogenesis and immune evasion: insights from Ebola virus and Marburg virus. Nat. Rev. Microbiol. 13, 663-676 (2015).
160. B. Meyer, H. Ly, Inhibition of Innate Immune Responses Is Key to Pathogenesis by Arenaviruses. J. Virol. 90, 3810-3818 (2016).
161. H. Murphy, H. Ly, Understanding Immune Responses to Lassa Virus Infection and to Its Candidate Vaccines. Vaccines (Basel) 10, (2022).
162. R. F. Garry, Lassa fever—the road ahead. Nat. Rev. Microbiol. 21, 87-96 (2023).
163. E. H. Jho et al., Wnt/beta-catenin/Tcf signaling induces the transcription of Axin2, a negative regulator of the signaling pathway. Mol. Cell. Biol. 22, 1172-1183 (2002).
164. I. J. Huggins et al., The WNT target SP5 negatively regulates WNT transcriptional programs in human pluripotent stem cells. Nat Commun 8, 1034 (2017).
165. J. Yang et al., Beta-catenin signaling in murine liver zonation and regeneration: A Wnt-Wnt situation! Hepatology 60, 964-976 (2014).
166. K. Pakos-Zebrucka et al., The integrated stress response. EMBO Rep 17, 1374-1395 (2016).
167. M. Costa-Mattioli, P. Walter, The integrated stress response: From mechanism to disease. Science 368, (2020).
168. J. M. Replogle et al., Mapping information-rich genotype-phenotype landscapes with genome-scale Perturb-seq. Cell 185, 2559-2575 e2528 (2022).
169. B. Adamson et al., A Multiplexed Single-Cell CRISPR Screening Platform Enables Systematic Dissection of the Unfolded Protein Response. Cell 167, 1867-1882.e1821 (2016).
170. P. Nell et al., Identification of an FXR-modulated liver-intestine hybrid state in iPSC-derived hepatocyte-like cells. J Hepatol 77, 1386-1398 (2022).
171. M. Belanger, I. Allaman, P. J. Magistretti, Brain energy metabolism: focus on astrocyte-neuron metabolic cooperation. Cell Metab 14, 724-738 (2011).
172. M. Stumvoll et al., Uptake and release of glucose by the human kidney. Postabsorptive rates and responses to epinephrine. J. Clin. Invest. 96, 2528-2533 (1995).
173. E. D. Rosen, B. M. Spiegelman, Adipocytes as regulators of energy balance and glucose homeostasis. Nature 444, 847-853 (2006).
174. D. G. Drubin, A. A. Hyman, Stem cells: the new “model organism”. Molecular Biology of the Cell 28, 1409-1411 (2017).
175. L. T. Ang et al., Generating human artery and vein cells from pluripotent stem cells highlights the arterial tropism of Nipah and Hendra viruses. Cell 185, 2523-2541 e2530 (2022).
176. J. L. Fowler, L. T. ANG, K. M. Loh, A critical look: Challenges in differentiating human pluripotent stem cells into desired cell types and organoids. Wiley Interdisciplinary Reviews: Developmental Biology 9, 891 (2020).
177. N. Sharma, M. S. Cappell, Gastrointestinal and Hepatic Manifestations of Ebola Virus Infection. Dig. Dis. Sci. 60, 2590-2603 (2015).
178. L. Baseler, D. S. Chertow, K. M. Johnson, H. Feldmann, D. M. Morens, The Pathogenesis of Ebola Virus Disease. Annu Rev Pathol 12, 387-418 (2017).
179. R. J. Schulze, M. B. Schott, C. A. Casey, P. L. Tuma, M. A. McNiven, The cell biology of the hepatocyte: A membrane trafficking machine. J Cell Biol 218, 2096-2112 (2019).
180. J. R. Spengler et al., Severity of Disease in Humanized Mice Infected With Ebola Virus or Reston Virus Is Associated With Magnitude of Early Viral Replication in Liver. J. Infect. Dis. 217, 58-63 (2017).
181. B. Escudero-Perez et al., Comparative pathogenesis of Ebola virus and Reston virus infection in humanized mice. JCI Insight 4, (2019).
182. P. S. Sung, E. C. Shin, Interferon Response in Hepatitis C Virus-Infected Hepatocytes: Issues to Consider in the Era of Direct-Acting Antivirals. Int J Mol Sci 21, (2020).
183. A. J. Ozga, M. T. Chow, A. D. Luster, Chemokines and the immune response to cancer. Immunity 54, 859-874 (2021).
184. C. J. Peters, C. T. Liu, G. W. Anderson, Jr., J. C. Morrill, P. B. Jahrling, Pathogenesis of viral hemorrhagic fevers: Rift Valley fever and Lassa fever contrasted. Rev Infect Dis 11 Suppl 4, S743-749 (1989).
185. R. W. Cross et al., Comparison of the Pathogenesis of the Angola and Ravn Strains of Marburg Virus in the Outbred Guinea Pig Model. J. Infect. Dis. 212 Suppl 2, S258-270 (2015).
186. K. J. Lavender et al., Pathogenicity of Ebola and Marburg Viruses Is Associated With Differential Activation of the Myeloid Compartment in Humanized Triple Knockout-Bone Marrow, Liver, and Thymus Mice. J. Infect. Dis. 218, S409-S417 (2018).
187. E. Muhlberger, M. Weik, V. E. Volchkov, H. D. Klenk, S. Becker, Comparison of the transcription and replication strategies of marburg virus and Ebola virus by using artificial replication systems. J. Virol. 73, 2333-2342 (1999).
188. M. R. Edwards et al., Differential Regulation of Interferon Responses by Ebola and Marburg Virus VP35 Proteins. Cell Reports 14, 1632-1640 (2016).
189. J. C. Guito, C. G. Albarino, A. K. Chakrabarti, J. S. Towner, Novel activities by ebolavirus and marburgvirus interferon antagonists revealed using a standardized in vitro reporter system. Virology 501, 147-165 (2017).
190. J. K. Richmond, D. J. Baglole, Lassa fever: epidemiology, clinical features, and social consequences. BMJ 327, 1271-1275 (2003).
191. D. W. Hawman, H. Feldmann, Crimean-Congo haemorrhagic fever virus. Nat. Rev. Microbiol. 21, 463-477 (2023).
192. M. E. Lindquist et al., Exploring Crimean-Congo Hemorrhagic Fever Virus-Induced Hepatic Injury Using Antibody-Mediated Type I Interferon Blockade in Mice. J. Virol. 92, (2018).
193. S. S. Rathore et al., Crimean-Congo haemorrhagic fever-induced liver injury: A systematic review and meta-analysis. Int. J. Clin. Pract. 75, e14775 (2021).
194. R. Rodrigues, G. Paranhos-Baccala, G. Vernet, C. N. Peyrefitte, Crimean-Congo hemorrhagic fever virus-infected hepatocytes induce ER-stress and apoptosis crosstalk. PLoS One 7, e29712 (2012).
195. J. I. Maiztegui et al., Ultrastructural and immunohistochemical studies in five cases of Argentine hemorrhagic fever. J Infect Dis 132, 35-53 (1975).
196. T. Sun et al., Axin2+ Pericentral Hepatocytes Have Limited Contributions to Liver Homeostasis and Regeneration. Cell Stem Cell 26, 97-107.e106 (2020).
197. B. Wang, L. Zhao, M. Fish, C. Y. Logan, R. Nusse, Self-renewing diploid Axin2+ cells fuel homeostatic renewal of the liver. Nature 524, 180-185 (2015).
198. F. Chen et al., Broad Distribution of Hepatocyte Proliferation in Liver Homeostasis and Regeneration. Cell Stem Cell 26, 27-33.e24 (2020).
199. W. Pu et al., Mfsd2a+ hepatocytes repopulate the liver during injury and regeneration. Nature Communications 7, 13369 (2016).
200. X. Han et al., Lineage Tracing Reveals the Bipotency of SOX9(+) Hepatocytes during Liver Regeneration. Stem Cell Reports 12, 624-638 (2019).
201. Y. Wang et al., Genetic tracing of hepatocytes in liver homeostasis, injury, and regeneration. Journal of Biological Chemistry 292, 8594-8604 (2017).
202. R. P. Davis et al., Targeting a GFP reporter gene to the MIXL1 locus of human embryonic stem cells identifies human primitive streak-like cells and enables isolation of primitive hematopoietic precursors. Blood 111, 1876-1884 (2008).
203. H. M. T. Choi et al., Third-generation in situ hybridization chain reaction: multiplexed, quantitative, sensitive, versatile, robust. Development 145, dev165753-165122 (2018).
204. T. Stuart et al., Comprehensive Integration of Single-Cell Data. Cell 177, 1888-1902.e1821 (2019).
205. E. Normandin et al., Natural history of Ebola virus disease in rhesus monkeys shows viral variant emergence dynamics and tissue-specific host responses. Cell Genom 3, 100440 (2023).

Materials and Methods

| | | |
|---|---|---|
| Reagent or Resource | Source | Identifier |

Antibodies

| | | |
|---|---|---|
| Anti-CD200-APC | Biolegend, 329208 | 5 μL per 10⁶ cells in 100 μL |
| Anti-ASGR1-PE | BD Biosciences, 563655 | 5 μL per 10⁶ cells in 100 μL |
| Anti-ALBUMIN-APC | R&D Systems, IC1455A, 200 μg/mL | 0.132 μg per 250k cells |
| Anti-ALBUMIN | Abcam, ab2406 | 1:1000 |
| Anti-CDX2 | R&D Systems, AF3665, 200 μg/mL | 1:100 |
| Anti-PDX1 | R&D Systems, AF2419, 200 μg/mL | 1:100 |
| Anti-CYP3A4 | Thermo Fisher, MA5-17064 | 1:200 |
| Anti-GLUL | Abcam, ab16802 | 1:1000 |
| Anti-FAH | Applied Biological Materials, | 3 μg/ml |
| | Y123010 | |
| APC Donkey Anti-Goat | R&D Systems | F0108 (1:10) |
| Donkey Serum | Jackson ImmunoResearch | 017-000-121 |
| Alexa Fluor 568 Donkey Anti- | Thermo Fisher Scientific | 1:250 |
| rabbit | | |
| Alexa Fluor 647 Donkey Anti- | Thermo Fisher Scientific | 1:500 |
| rabbit | | |
| Alexa Fluor 647 Donkey Anti- | Thermo Fisher Scientific | 1:500 |
| mouse | | |

Bacterial and viral strains

| | | |
|---|---|---|
| Ebola virus, GFP-expressing | NIH National Institute of Allergy and | (Hoenen et al., 2013) |
| (Ebola virus/Human- | Infectious Diseases, Rocky | |
| recombinant/COD/1976/Yam | Mountain Laboratories | |
| buku-Mayinga-GFP) | | |
| Sudan virus, zsGreen- | Centers for Disease Control and | (unpublished, CDC VSPB) |
| expressing (isolate | Prevention, Viral Special Pathogens | |
| designation unknown) | Branch | |
| Marburg virus, zsGreen- | Centers for Disease Control and | (Albarino et al., 2018) |
| expressing (Marburg | Prevention, Viral Special Pathogens | |
| virus/Bat- | Branch | |
| recombinant/Uganda/2007/Ba | | |
| t371-zsGreen) | | |
| Lassa virus, zsGreen- | Centers for Disease Control and | (Welch et al., 2016) |
| expressing (Lassa | Prevention, Viral Special Pathogens | |
| virus/Mouse- | Branch | |
| recombinant/Sierra | | |
| Leone/1976/Josiah) | | |
| Sendai virus (Cantell strain) | ATCC | VR-907 |
| Sendai virus (recombinant, | Dr. Mahito Nakanishi, National | (Nishimura et al., 2017) |
| replication-defective, | Institute of Advanced Industrial | |
| encoding KLF4/OCT4/SOX2/ | Science and Technology | |
| MYC and miR-302 target site) | | |
| NEB ® Stable Competent | New England BioLabs | C3040H |
| E. coli | | |

Chemicals, peptides and recombinant proteins

| | | |
|---|---|---|
| mTesR Plus medium | STEMCELL Technologies | #100-0276 |
| Penicillin/streptomycin | Thermo Fisher Scientific | 15-140-122 |
| KnockOut Serum | Thermo Fisher Scientific | 10828028 |
| Replacement | | |
| MEM Non-Essential Amino | Thermo Fisher Scientific | 11-140-050 |
| Acids Solution (100X) | | |
| Geltrex LDEV-Free, hESC- | Thermo Fisher Scientific | A1413302 |
| Qualified, Reduced Growth | | |
| Factor Basement Membrane | | |
| Matrix | | |
| Recombinant Human | Gibco | A14700 |
| Vitronectin Protein, Truncated | | |
| (VTN-N) | | |
| Matrigel Matrix GFR Phenol | BD Biosciences | 356231 |
| RF | | |
| Gelatin Solution | Sigma Aldrich | ES-006-B |
| Versene solution | Thermo Fisher Scientific | 15040066 |
| Trypsin-EDTA, 0.05% | Thermo Fisher Scientific | 25300054 or 25300062 |
| Accutase - Enzyme Cell | Thermo Fisher Scientific | 00-4555-56 |
| Detachment Medium | | |
| TrypLE Express Enzyme (1X) | Thermo Fisher Scientific | 12604013 |
| Fetal Bovine Serum Premium | Atlanta Biologicals | S11550H |
| Select | | |
| 2-mercaptoethanol | Gibco | 21985023 |
| DMEM | Sigma Aldrich | D6429 |
| DMEM/F12 + GlutaMAX | Gibco | 10565042 |
| IMDM + GlutaMAX | Thermo Fisher Scientific | 31980-097 |
| F12 + GlutaMAX | Thermo Fisher Scientific | 31765-092 |
| Polyvinyl alcohol | Sigma Aldrich | P8136-250G |
| Chemically defined lipid | Thermo Fisher Scientific | 11905-031 |
| concentrate | | |
| 1-Thioglycerol | Sigma Aldrich | M6145-100ML |
| Recombinant human insulin | Sigma Aldrich | 11376497001 |
| Human transferrin | Sigma Aldrich | 10652202001 |
| Recombinant Activin | R&D Systems | 338-AC-500/CF |
| Recombinant human BMP4 | R&D Systems | 314-BP-050 |
| Recombinant human FGF2 | PeproTech | 10018B |
| Penicillin/streptomycin | Thermo Fisher Scientific | 15070-063 |
| PI-103 | Tocris Bioscience | 2930 |
| LDN-193189 dihydrochloride | Tocris Bioscience | 605310 |
| A83-01 | Tocris Bioscience | 2939 |
| TTNPB | Tocris Bioscience | 0761 |
| Recombinant Oncostatin M | R&D Systems | 295OM050 |
| Dexamethasone | Tocris Bioscience | 1126 |
| Thiazovivin | Tocris Bioscience | 3845 |
| Forskolin | Tocris Bioscience | 1099 |
| Ascorbic acid-2-phosphate | Sigma Aldrich | 49752-10G |
| RO4929097 | Cellagen Technology | C7649-10 |
| CHIR99201 | Tocris | 4423 |
| 8-bromo-cAMP | Tocris | 1140 |
| L-Ascorbic acid-2-phosphate | Cayman Chemical | 16457 |
| Bovine serum albumin (BSA) | Sigma Aldrich | A2153 |
| Tween-20 | Sigma Aldrich | P9416 |
| 10% Formalin | Sigma Aldrich | F5554 |
| Vectashield | Vector Labs | H-100 |
| Glutamax | Gibco | 35050061 |
| Poly(I:C) sodium salt | Sigma Aldrich | P1530 |
| NTBC | Yecuris | 20-0027 |
| PicoLab High energy mouse | Newco Distribution Inc | 5LJ5 |
| diet | | |
| Human cryopreserved | Lonza Group | MCHT50 |
| hepatocyte thawing medium | | |
| Cryopreserved Hepatocytes | Gibco | CHRM |
| Recovery Medium | | |
| HCM BulletKit | Lonza Group | CC-3199, CC-4182, CC-3198 |
| Critical commercial assays | | |
| QIAamp Viral RNA kit | Qiagen | 52904 |
| Quick-RNA microprep kit | Zymo Research | R1051 |
| High-Capacity cDNA Reverse | Applied Biosystems | 4368814 |
| Transcription Kit | | |
| SYBRGreen | Thomas Scientific | BIO-94050 |
| 10x Genomics Chromium | 10x Genomics | 1000075 |
| single cell 3′ GEM reagent kit | | |
| (v3) | | |
| Chromium Chip B Single Cell | 10x Genomics | 1000073 |
| Kit 48rxns | | |
| LEGENDScreen PE- | Biolegend | 700007 |
| Conjugated Human Antibody | | |
| Plates | | |
| Triton X-100 | Sigma Aldrich | X100-500ML |
| PBS, pH 7.4 | Thermo Fisher | 10010049 |
| DPBS without ions | Thermo Fisher | 14190144 |
| Rat tail collagen 1 coated | Corning | 354236 |
| plates | | |
| Live/Dead viability/cytotoxicity | Thermo Fisher Scientific | L3224 |
| kit. | | |
| ATP Assay Kit | Abcam | ab83355 |
| Triglyceride Assay Kit | Zen-Bio Inc | TG-1-NC |
| Urea Assay Kit | Abcam | ab83362 |
| Glycogen Assay Kit II | Abcam | ab169558 |
| Oil Red O stain solution | Lifeline Cell Technology | LL-0052 |
| Molecular Probes DAPI (4′,6 | Thermo Fisher Scientific | D1306 |
| Diamidino 2 Phenylindole, | | |
| Dihydrochloride) | | |
| Prolong Gold Antifade | Thermo Fisher Scientific | P10144 |
| Mountant | | |
| Experimental models: cell | | |
| lines | | |
| H1 hESCs | WiCell Research Institute | WiCell, WA01 |
| H7 hESCs | WiCell Research Institute | WiCell, WA07 |
| H9 hESCs | WiCell Research Institute | WiCell, WA09 |
| Hepatosure pooled human | Sekisui XenoTech | HCP100.H15 LOT: 2010012 |
| hepatocytes | | |
| Human cryopreserved | Lonza Group | HUCPG |
| hepatocytes, plateable | | |
| Hep10, pooled cryopreserved | Gibco | HMCS10 |
| human hepatocytes | | |
| HepG2 cell line | Gift from Ren Ee Chee | NA |
| HuH7 human liver cancer cell | Sekisui XenoTech | JCRB0403 |
| line | | |
| Vero C1008 (Vero 76, clone | ECACC | 85020206 |
| E6, Vero E6) | | |

Experimental models: organisms/strains

| | | |
|---|---|---|
| Mus musculus (mouse): NOD- | The Jackson Laboratory | 005557 |
| SCID Il2rg^{-/-} strain | | |
| Mus musculus (mouse): FRG | Yecuris Corporation | 10-0008 |
| FAH^{-/-}Rag2^{-/-}IL2Rg^{-/-} NOD | | |
| strain | | |

Software and algorithms

| | |
|---|---|
| Seurat | Stuart et al., 2019 (Satija lab) |
| Image J/Fiji | Schindelin et al., 2012 |
| Ggplot | Wickham H (2016) |
| Tidyverse | Wickham H (2019) |
| Dplyr | Wickham H (2021) |
| Limma | Ritchie et al. 2015 |
| Voom | Law et al., 2014 |
| RStudio | RStudio team |
| Encode ATAC-seq pipeline | Kundaje lab |
| Kallisto | Pachter lab |
| Cell Ranger | 10x genomics |
| DESEQ2 | Love et al., 2014 |
| Homer | Heinz et al., 2010 (Benner Lab) |
| Diffbind | Stark & Brown (2011) |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method to select for metabolically selectable mammalian cells that don't require exogenous glucose and glutamine in culture, the method comprising:
culturing a mixed population of cells comprising metabolically selectable mammalian cells and non-metabolically selectable cells in medium substantially free of glucose and glutamine for a period of time sufficient to kill non-metabolically selectable mammalian cells in the culture.

2. The method of claim 1, wherein the medium is further substantially free of pyruvate.

3. The method of claim 1, wherein the metabolically selectable cells comprise a cell type selected from the group consisting of hepatocytes, astrocytes, and smooth muscle cells.

4. The method of claim 3, wherein the metabolically selectable cells are hepatocytes.

5. The method of claim 4, wherein the hepatocytes comprise cells that express one or more markers selected from the group consisting of ALB, FGB, SERPINA1, CPS1, ARG1, TTR, FAH, and AAT.

6. The method of claim 1, wherein the concentration of glucose, pyruvate and glutamine in the culture medium is less than about 10 μM.

7. The method of claim 1, wherein the medium comprises HepSelect medium comprising 100% Dulbecco's Modified Eagle Medium (DMEM) substantially lacking glucose, glutamine, phenol red, and pyruvate, transferrin, penicillin and streptomycin.

8. The method of claim 7 wherein the medium further comprises insulin, PKA agonist (Forskolin), Ascorbic-2-phosphate, Glucocorticoid receptor agonist (Dexamethasone), Notch inhibitor (Ro4929097), Transferrin.

9. The method of claim 1, wherein the period of time is at least about 24 hours.

10. The method of claim 1, wherein the mammalian cells are human cells.

11. The method of claim 1, wherein the mixed population of cells comprising metabolically selectable mammalian cells and non-metabolically selectable cells, comprises human hepatocytes derived from pluripotent stem cells in culture.

12. A culture medium substantially free of glucose and glutamine, comprising a mixed population of cells comprising metabolically selectable mammalian cells and non-metabolically selectable mammalian cells.

13. The medium of claim 12, wherein the medium is further substantially free of pyruvate.

14. The medium of claim 12, wherein the metabolically selectable mammalian cells are selected from astrocytes, smooth muscle cells, and hepatocytes.

15. The medium of claim 14, wherein the metabolically selectable mammalian cells are hepatocytes.

16. The medium of claim 12, wherein the concentration of glucose, pyruvate and glutamine in the culture medium is less than about 10 μM.
